SEQUENCE LISTING (58 sequences)

SEQ ID NO: 1 - protein, 4 aa, Artificial
  Synthetic Construct
VPGG

SEQ ID NO: 2 - protein, 4 aa, Artificial
  Synthetic Construct
IPGG

SEQ ID NO: 3 - protein, 5 aa, Artificial
  Synthetic Construct
VPGXG

SEQ ID NO: 4 - protein, 5 aa, Artificial
  Synthetic Construct
AVGVP

SEQ ID NO: 5 - protein, 5 aa, Artificial
  Synthetic Construct
IPGVG

SEQ ID NO: 6 - protein, 5 aa, Artificial
  Synthetic Construct
LPGVG

SEQ ID NO: 7 - protein, 6 aa, Artificial
  Synthetic Construct
VAPGVG

SEQ ID NO: 8 - protein, 8 aa, Artificial
  Synthetic Construct
GVGVPGVG

SEQ ID NO: 9 - protein, 9 aa, Artificial
  Synthetic Construct
VPGFGVGAG

SEQ ID NO: 10 - protein, 9 aa, Artificial
  Synthetic Construct
VPGVGVPGG

SEQ ID NO: 11 - protein, 5 aa, Artificial
  Synthetic Construct
IPGXG

SEQ ID NO: 12 - protein, 5 aa, Artificial
  Synthetic Construct
LPGXG

SEQ ID NO: 13 - protein, 50 aa, Artificial
  Synthetic Construct
VPGVGVPGVGVPGVGVPGVGVPGVGVPGAGVPGAGVPGGGVPGGGVPGGG

SEQ ID NO: 14 - protein, 25 aa, Artificial
  Synthetic Construct
VPGVGVPGVGVPGVGVPGVGVPGVG

SEQ ID NO: 15 - DNA, 75 bp, Artificial
  Synthetic Construct
gtgggtgttccgggcgtaggtgtcccaggtgtgggcgtaccgggcgttggtgttcctggtgtcggcgtgccgggc

SEQ ID NO: 16 - protein, 450 aa, Artificial
  Synthetic Construct
VPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGGVPGVGVPGVGVPGVGVPGVGVPGVGVPGAGVPGAGVPGGGVPGGGVPGGG

SEQ ID NO: 17 - protein, 100 aa, Artificial
  Synthetic Construct
VPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVG

SEQ ID NO: 18 - protein, 20 aa, Artificial
  Synthetic Construct
VPGKGVPGVGVPGVGVPGFG

SEQ ID NO: 19 - protein, 45 aa, Artificial
  Synthetic Construct
VPGKGVPGVGVPGVGVPGVGVPGVGVPGVGVPGVGVPGVGVPGFG

SEQ ID NO: 20 - protein, 25 aa, Artificial
  Synthetic Construct
AVGVPAVGVPAVGVPAVGVPAVGVP

SEQ ID NO: 21 - protein, 25 aa, Artificial
  Synthetic Construct
IPGVGIPGVGIPGVGIPGVGIPGVG

SEQ ID NO: 22 - protein, 25 aa, Artificial
  Synthetic Construct
LPGVGLPGVGLPGVGLPGVGLPGVG

SEQ ID NO: 23 - protein, 339 aa, Artificial
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASSGTDDDDKGIVEQCCTSICSLYQLENYCN

SEQ ID NO: 24 - protein, 489 aa, Artificial
  Synthetic Construct
MGGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPGASSGTDDDDKGIVEQCCTSICSLYQLENYCN

SEQ ID NO: 25 - protein, 639 aa, Artificial
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASSGTDDDDKGIVEQCCTSICSLYQLENYCN

SEQ ID NO: 26 - protein, 939 aa, Artificial
  Synthetic Construct
MGGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPGASSGTDDDDKGIVEQCCTSICSLYQLENYCN

SEQ ID NO: 27 - protein, 354 aa, Artificial
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASSGTDDDDKYTSLIHSLIEESQNQQEKNEQELLELDKWASLWNWF

SEQ ID NO: 28 - protein, 504 aa, Artificial
  Synthetic Construct
MGGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPGASSGTDDDDKYTSLIHSLIEESQNQQEKNEQELLELDKWASLWNWF

SEQ ID NO: 29 - protein, 654 aa, Artificial
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASSGTDDDDKYTSLIHSLIEESQNQQEKNEQELLELDKWASLWNWF

SEQ ID NO: 30 - protein, 357 aa, Artificial
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASGGGGPLVPRGSYTSLIHSLIEESQNQQEKNEQELLELDKWASLWNWF

SEQ ID NO: 31 - protein, 507 aa, Artificial
  Synthetic Construct
MGGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPGASGGGGPLVPRGSYTSLIHSLIEESQNQQEKNEQELLELDKWASLWNWF

SEQ ID NO: 32 - protein, 657 aa, Artificial
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASGGGGPLVPRGSYTSLIHSLIEESQNQQEKNEQELLELDKWASLWNWF

SEQ ID NO: 33 - protein, 357 aa, Artificial
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASGPTTENLYFQSYTSLIHSLIEESQNQQEKNEQELLELDKWASLWNWF

SEQ ID NO: 34 - protein, 507 aa, Artificial
  Synthetic Construct
MGGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPGASGPTTENLYFQSYTSLIHSLIEESQNQQEKNEQELLELDKWASLWNWF

SEQ ID NO: 35 - protein, 657 aa, Artificial
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASGPTTENLYFQSYTSLIHSLIEESQNQQEKNEQELLELDKWASLWNWF

SEQ ID NO: 36 - protein, 356 aa, Artificial
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASGPTTENLYFQYTSLIHSLIEESQNQQEKNEQELLELDKWASLWNWF

SEQ ID NO: 37 - protein, 506 aa, Artificial
  Synthetic Construct
MGGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPGASGPTTENLYFQYTSLIHSLIEESQNQQEKNEQELLELDKWASLWNWF

SEQ ID NO: 38 - protein, 656 aa, Artificial
  Synthetic Construct
MGGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPGASGPTTENLYFQYTSLIHSLIEESQNQQEKNEQELLELDKWASLWNWF

SEQ ID NO: 39 - protein, 669 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGLVPRGSPGISGGGGGHMPMALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 40 - protein, 574 aa, Artificial
  Synthetic Construct
MRALMGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPSSGLVPRGSPGISGGGGGHMPMGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 41 - protein, 724 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGLVPRGSPGISGGGGGHMPMGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 42 - protein, 874 aa, Artificial
  Synthetic Construct
MRALMGPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWPSSGLVPRGSPGISGGGGGHMPMGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 43 - protein, 1174 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGLVPRGSPGISGGGGGHMPMGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 44 - protein, 735 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMSTSQPGACPCQGAASRPAILYALLSSSLKAVPRPRSRCLCRQHRPVQLCAPHRTCREALDVLAKTVAFLRNLPSFWQLPPQDQRRLLQGCWGPLFLLGLAQDAVTFEVAEAPVPSILKKILLEEPSSSGGSGQLPDRPQPSLAAVQWLQCCLESFWSLELSPKEYACLKGTILFNPDVPGLQAASHIGHLQQEAHWVLCEVLEPWCPAAQGRLTRVLLTASTLKSIPTSLLGDLFFRPIIGDVDIAGLLGDMLLLR

SEQ ID NO: 45 - protein, 736 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 46 - protein, 1186 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 47 - protein, 757 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMIQQATTGVSQETSENPGDKTIVPATLPQLTPTLVSLLEVIEPEVLYAGYDSSVPDSTWRIMTTLNMLGGRQVIAAVKWAKAIPGFRNLHLDDQMTLLQYSWMSLMAFALGWRSYRQSSANLLCFAPDLIINEQRMTLPDMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 48 - protein, 624 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 49 - protein, 774 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 50 - protein, 1225 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGLVPRGSPGISGGGGGHMSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 51 - protein, 775 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGDYDIPTTENLYFQGAHMSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 52 - protein, 859 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGSIGPLVPRGSHSMGLNDIFEAQKIEWHEHMPMALEMTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKINYSHFTCATDTENIRFVFAAVKDTILQLNLKEYNLV

SEQ ID NO: 53 - protein, 1309 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGSIGPLVPRGSHSMGLNDIFEAQKIEWHEHMPMALEMTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKINYSHFTCATDTENIRFVFAAVKDTILQLNLKEYNLV

SEQ ID NO: 54 - protein, 728 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMKQLTILGSTGSIGCSTLDVVRHNPEHFRVVALVAGKNVTRMVEQCLEFSPRYAVMDDEASAKLLKTMLQQQGSRTEVLSGQQAACDMAALEDVDQVMAAIVGAAGLLPTLAAIRAGKTILLANKESLVTCGRLFMDAVKQSKAQLLPVDSEHNAIFQSLPQPIQHNLGYADLEQNGVVSILLTGSGGPFRETPLRDLATMTPDQACRHPNWSMGRKISVDSATMMNKGLEYIEARWLFNASASQMEVLIHPQSVIHSMVRYQDGSVLAQLGEPDMRTPIAHTMAWPNRVNSGVKPLDFCKLSALTFAAPDYDRYPCLKLAMEAFEQGQAATTALNAANEITVAAFLAQQIRFTDIAALNLSVLEKMDMREPQCVDDVLSVDASAREVARKEVMRLASPV

SEQ ID NO: 55 - protein, 879 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGLVPRGSPGISGGGGGHMKQLTILGSTGSIGCSTLDVVRHNPEHFRVVALVAGKNVTRMVEQCLEFSPRYAVMDDEASAKLLKTMLQQQGSRTEVLSGQQAACDMAALEDVDQVMAAIVGAAGLLPTLAAIRAGKTILLANKESLVTCGRLFMDAVKQSKAQLLPVDSEHNAIFQSLPQPIQHNLGYADLEQNGVVSILLTGSGGPFRETPLRDLATMTPDQACRHPNWSMGRKISVDSATMMNKGLEYIEARWLFNASASQMEVLIHPQSVIHSMVRYQDGSVLAQLGEPDMRTPIAHTMAWPNRVNSGVKPLDFCKLSALTFAAPDYDRYPCLKLAMEAFEQGQAATTALNAANEITVAAFLAQQIRFTDIAALNLSVLEKMDMREPQCVDDVLSVDASAREVARKEVMRLASPV

SEQ ID NO: 56 - protein, 1329 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGLVPRGSPGISGGGGGHMKQLTILGSTGSIGCSTLDVVRHNPEHFRVVALVAGKNVTRMVEQCLEFSPRYAVMDDEASAKLLKTMLQQQGSRTEVLSGQQAACDMAALEDVDQVMAAIVGAAGLLPTLAAIRAGKTILLANKESLVTCGRLFMDAVKQSKAQLLPVDSEHNAIFQSLPQPIQHNLGYADLEQNGVVSILLTGSGGPFRETPLRDLATMTPDQACRHPNWSMGRKISVDSATMMNKGLEYIEARWLFNASASQMEVLIHPQSVIHSMVRYQDGSVLAQLGEPDMRTPIAHTMAWPNRVNSGVKPLDFCKLSALTFAAPDYDRYPCLKLAMEAFEQGQAATTALNAANEITVAAFLAQQIRFTDIAALNLSVLEKMDMREPQCVDDVLSVDASAREVARKEVMRLASPV

SEQ ID NO: 57 - protein, 879 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGDYDIPTTENLYFQGAHMKQLTILGSTGSIGCSTLDVVRHNPEHFRVVALVAGKNVTRMVEQCLEFSPRYAVMDDEASAKLLKTMLQQQGSRTEVLSGQQAACDMAALEDVDQVMAAIVGAAGLLPTLAAIRAGKTILLANKESLVTCGRLFMDAVKQSKAQLLPVDSEHNAIFQSLPQPIQHNLGYADLEQNGVVSILLTGSGGPFRETPLRDLATMTPDQACRHPNWSMGRKISVDSATMMNKGLEYIEARWLFNASASQMEVLIHPQSVIHSMVRYQDGSVLAQLGEPDMRTPIAHTMAWPNRVNSGVKPLDFCKLSALTFAAPDYDRYPCLKLAMEAFEQGQAATTALNAANEITVAAFLAQQIRFTDIAALNLSVLEKMDMREPQCVDDVLSVDASAREVARKEVMRLASPV

SEQ ID NO: 58 - protein, 864 aa, Artificial
  Synthetic Construct
MRALMGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWPSSGGGGGSIGPLVPRGSHMPMALEMGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

(19) United States
(12) Patent Application Publication
(10) Pub. No.: US 2005/0255554 A1
(43) Pub. Date: Nov. 17, 2005

(54) Fusion peptides isolatable by phase transition

(76) Inventor: Ashutosh Chilkoti, Durham, NC (US)

Correspondence Address: INTELLECTUAL PROPERTY / TECHNOLOGY LAW, PO BOX 14329, RESEARCH TRIANGLE PARK, NC, 27709, US

(21) Appl. No.: 11/053,100

(22) Filed: Feb. 8, 2005

Related U.S. Application Data
(63) Continuation-in-part of application No. 09/812,382, filed on Mar. 20, 2001, now Pat. No. 6,852,834.
(60) Provisional application No. 60/190,659, filed on Mar. 20, 2000.

(51) Int. Cl. A61K 38/17; C07H 21/04; C12P 21/06; C12N 9/00; C12N 15/09

(52) U.S. Cl. 435/69.1; 530/350; 514/12; 435/320.1; 435/325; 536/23.5; 435/183

(57) ABSTRACT

Genetically-encodable, environmentally-responsive fusion proteins comprising ELP peptides. Such fusion proteins exhibit unique physico-chemical and functional properties that can be modulated as a function of solution environment. The invention also provides methods for purifying the FPs, which take advantage of these unique properties, including high-throughput purification methods.

### CROSS-REFERENCE TO RELATED APPLICATION

This is a continuation-in-part of U.S. patent application Ser. No. 09/812,382 filed on Mar. 20, 2001 in the name of Ashutosh Chilkoti and entitled “FUSION PEPTIDES ISOLATABLE BY PHASE TRANSITION,” which in turn claims priority to U.S. Provisional Patent Application No. 60/190,659 filed Mar. 20, 2000.

### GOVERNMENT RIGHTS IN INVENTION

Work relating to the invention was supported in part by grants from the National Institutes of Health (IR21-GM-057373-01 and RO1-GM-61232). The U.S. Government may have certain rights in the invention.

### BACKGROUND OF THE INVENTION

1. Field of the Invention

The invention provides a new generation of genetically-encodable, environmentally-responsive fusion proteins comprising elastin-like peptides (ELPs). The fusion proteins of the invention (referred to herein as “FPs”) exhibit unique physico-chemical and functional properties that can be modulated as a function of solution environment. The invention also provides methods for purifying the FPs, including high-throughput purification techniques, which take advantage of these unique properties.

### BACKGROUND OF THE INVENTION

Recombinant DNA techniques have facilitated the expression of proteins for diverse applications in medicine and biotechnology. However, the purification of recombinant proteins is often complicated and problematic. In the last decade, a number of protein expression systems have been developed to simplify protein purification. Such protein expression systems often operate by expressing a recombinant protein fused with a carrier protein or peptide. A number of fusion protein systems using different carrier proteins are now commercially available, particularly for E. coli expression. Examples include maltose binding protein, glutathione S-transferase, biotin carboxyl carrier protein, thioredoxin, and cellulose binding domain. Similarly, vectors that allow fusion of the target protein to short peptide tags such as oligohistidine, S-peptide, and the FLAG peptide are also available.

Fusion protein expression simplifies the separation of recombinant protein from cell extracts by one-step purification by affinity chromatography using an immobilized, moderate-affinity ligand specific to the carrier protein. Although useful for laboratory scale purification, the scale-up of affinity chromatography can represent a major cost of the final protein product at the preparative scale.

Additionally, chromatography represents a major bottleneck in high throughput purification of proteins. The full implications of the human genome project will not be realized until all the proteins encoded in the genome can be expressed and studied in detail. Current chromatographic technologies cannot be easily multiplexed to efficiently purify the wide diversity of proteins encoded in the human genome. These limitations of current bioseparation techniques, therefore, provide a compelling rationale for the development of non-chromatographic methods for the purification of soluble, recombinant proteins. Likewise, non-chromatographic purification methods would also be attractive as technically simple, reliable, and broadly applicable methods for bench top, milligram-scale purification of single proteins.

More economical and technically simple methods for purification of soluble proteins, which do not involve scale-up of chromatographic procedures, are therefore desirable.

### SUMMARY OF THE INVENTION

The inventor has surprisingly discovered that non-chromatographic, thermally-stimulated phase separation and purification of recombinant proteins can be achieved by forming fusion proteins that contain the target recombinant proteins with N- or C-terminal elastin-like polypeptide (ELP) tags.

ELPs are repeating peptide sequences that have been found to exist in the elastin protein. Among these repeating peptide sequences are polytetra-, polypenta-, polyhexa-, polyhepta-, polyocta, and polynonapeptides.

ELPs undergo a reversible inverse temperature transition: they are structurally disordered and highly soluble in water below a transition temperature (Tₜ), but exhibit a sharp (2-3° C. range) disorder-to-order phase transition when the temperature is raised above Tₜ, leading to desolvation and aggregation of the polypeptides. The ELP aggregates, when reaching sufficient size, can be readily removed and isolated from solution by centrifugation. More importantly, such phase transition is reversible, and the isolated ELP aggregates can be completely resolubilized in buffer solution when the temperature is returned below the Tₜ of the ELPs.

It was a surprising and unexpected discovery of the present invention that fusion proteins (“FPs”) containing target recombinant proteins with N- or C-terminal ELP tags also undergo a thermo-dependent phase transition similar to that of free ELPs.

This discovery is particularly useful for non-chromatographic, thermally-stimulated separation and purification of recombinant proteins. By fusing a thermally responsive ELP tag to a target protein of interest, environmentally sensitive solubility can be imparted to such target protein. In the practice of the present invention, the target proteins are expressed as soluble fusion proteins with N- or C-terminal ELP sequences in host organisms such as E. coli, wherein the fusion proteins exhibit a soluble-insoluble phase transition when the temperature is raised from below Tₜ to above Tₜ. This inverse phase transition is exploited in the process of the invention for purifying the target proteins from other soluble proteins produced by the organism, using a new nonchromatographic separation method, which the present inventor has termed “inverse transition cycling” (ITC).

The fundamental principle of ITC is remarkably simple. It involves forming an ELP fusion protein as described hereinabove, which contains the target protein with a N- or C-terminal ELP tag, rendering the ELP fusion protein insoluble in aqueous solution by triggering its inverse phase transition. This can be accomplished either by increasing the temperature above the Tₜ, or alternatively by depressing the Tₜ below the solution temperature by the addition of NaCl or other salt or solute, organic or inorganic, to the solution. This results in aggregation of the ELP fusion protein, allowing it to be collected by centrifugation or other weight- and/or size-dependent mass separation techniques, e.g., membrane separation or filtration. The aggregated ELP fusion protein can then be resolubilized in fresh buffer solution at a temperature below the Tₜ, thereby reversing the inverse phase transition, to yield soluble, functionally active, and purified fusion protein. Successive purification steps may also be carried out using ITC to achieve a highly pure, e.g., ultrapure, fusion protein product. Furthermore, ITC may also be used to concentrate and exchange buffers if desired as fgollows: the purified protein is aggregated by triggering the phase transition, and resolubilized in a smaller volume than before inducing the phase transion to concentrate the protein solution, and buffer excnage is achieved by simply resolubilizing the protein in a buffer of different composition than the starting buffer.

Free target protein then can be obtained, for example, by carrying out protease digestion or other scission process at an engineered recognition site located between the target protein and the ELP tag, followed by a final round of ITC to remove the cleaved ELP tag and yield the purified free target protein.

ITC has major advantages over other methods currently used for purification of recombinant proteins. It is technically simple, inexpensive, easily scaled up, and gentle, triggered by only modest alterations in temperature and/or ionic strength. The ITC technology is useful in the modulation of the physico-chemical properties of recombinant proteins and provides diverse applications in bioseparation, immunoassays, biocatalysis, and drug delivery.

The ITC methods of the invention exhibit significant advantages over currently used affinity purification methods in purifying recombinant fusion proteins. First, by circumventing chromatography, the expense associated with chromatographic resins and equipment is eliminated. Second, the separation and recovery conditions are gentle, requiring only a modest change in temperature or ionic strength. Third, the method is fast and technically simple, with only a few short centrifugation or filtration steps followed by resolubilization of the purified protein in a low ionic strength buffer. Finally, the equipment required, a temperature-controlled water bath and a centrifuge capable of operating at ambient temperature, are widely available. Additionally, ITC purification is independent of a specific expression vector or host and is exceptionally advantageous for use with eukaryotic expression systems, which readily over-express heterologous proteins in a soluble state.

The ITC methodology of the invention also addresses a compelling need in the art for high-throughput purification techniques. The ITC purification technique of the invention is scalable in character, and can be appropriately scaled and multiplexed for concurrent, parallel laboratory purifications from numerous cell cultures.

Simultaneous purification of proteins from multiple cultures using the ITC methodology of the invention enables expedited structure-function studies of proteins as well as screening of proteins in pharmaceutical studies.

The invention generally provides a fusion protein (FP) exhibiting a phase transition, the fusion protein comprising: (a) one or more biological molecules; (b) one or more proteins exhibiting a phase transition joined to the biologically active molecule; and (c) optionally, a spacer sequence separating any of the protein(s) of (b) from any of the biological molecule(s) of (a).

In a specific aspect, the fusion proteins of the invention constitute ELP fusion proteins, in which an ELP tag is bound to a protein of interest, as for example by direct bond linkage, or through an intermediate moiety therebetween. The intermediate moiety advantageously, in one embodiment of the invention, comprises a cleavage site that is cleavable by any suitable mechanism to yield the protein of interest subsequent to isolation/purification of the fusion protein. Cleavage mechanisms, discussed more fully hereinafter, encompass all means, methods and agents that are usefully employed to separate the fusion protein into its ITC-mediating portion and its protein of interest. The protein of interest can be of any suitable type, and encompasses a wide variety of protein components, including polypeptide therapeutic agents, prodrug agents, catalytic or reactant agents, etc. and the protein of interest can be produced in the fusion protein with ancillary protein moieties, including signal proteins for mediating cellular secretion of the protein product, heat shock proteins, etc.

Although discussed hereinafter primarily with reference to FPs comprising ELP components, it will be appreciated that other FPs, comprising other inverse phase transition-modulating components, are contemplated within the broad scope of the present invention. Nonetheless, the preferred practice of the invention relates to FPs comprising ELP carriers.

The inventor has surprisingly discovered that such FPs retain the inverse transition behavior of the ELP carrier. The FPs thus provide a new generation of genetically-encodable, environmentally-responsive proteins whose physico-chemical and functional properties can be modulated as a function of the solution environment. The inverse transition behavior of the FPs enables a one-step phase separation method for separating FPs from other soluble proteins.

The biological molecule component of the FP is preferably selected from the group consisting of proteins, lipids, carbohydrates, and single or double stranded oligonucleotides. More preferably, the biological molecule component comprises a polypeptide protein, most preferably a biologically active polypeptide, e.g., a therapeutic peptide, protein or an enzyme useful in industrial biocatalysis. The biological molecule component may also comprise a ligand-binding protein or an active fragment thereof, such as an antibody or antibody fragment, which has specific affinity for a protein of interest. Upon binding to the protein of interest, the fusion protein preferably retains some or all of its phase transition character, so that the protein of interest bound to such fusion protein may be isolated by inverse phase transition.

In addition to such biological molecule component, the FPs of the present invention further comprise one or more proteins exhibiting a phase transition. These proteins may be of any suitable type. Phase transition proteins usefully employed in the practice of the present invention include proteins exhibiting a β-turn structure, though such a structure is not strictly necessary, and other proteins devoid of β-turn structure and exhibiting a phase transition are advantageously utilized in protein purification and other applications of the present invention.

Specifically, the phase transition proteins of the present invention may comprise ELPs formed of polymeric or polymeric or oligomeric repeats of various characteristic tetra-, penta-, hexa-, hepta-, octa-, and nonapeptides, which include but are not limited to:
(a) tetrapeptide Val-Pro-Gly-Gly, or VPGG (SEQ ID NO: 1);
(b) tetrapeptide Ile-Pro-Gly-Gly, or IPGG (SEQ ID NO: 2);
(c) pentapeptide Val-Pro-Gly-X-Gly (SEQ ID NO: 3), or VPGXG, wherein X is any natural or non-natural amino acid residue, and wherein X optionally varies among polymeric or oligomeric repeats;
(d) pentapeptide Ala-Val-Gly-Val-Pro, or AVGVP (SEQ ID NO: 4);
(e) pentapeptide Ile-Pro-Gly-Val-Gly, or IPGVG (SEQ ID NO: 5);
(f) pentapeptide Leu-Pro-Gly-Val-Gly, or LPGVG (SEQ ID NO: 6);
(g) hexapeptide Val-Ala-Pro-Gly-Val-Gly, or VAPGVG (SEQ ID NO: 7);
(h) octapeptide Gly-Val-Gly-Val-Pro-Gly-Val-Gly, or GVGVPGVG (SEQ ID NO: 8);
(i) nonapeptide Val-Pro-Gly-Phe-Gly-Val-Gly-Ala-Gly, or VPGFGVGAG (SEQ ID NO: 9); and
(j) nonapeptides Val-Pro-Gly-Val-Gly-Val-Pro-Gly-Gly, or VPGVGVPGG (SEQ ID NO: 10).

Other polymeric or oligomeric repeat units of varying size and constitution are also usefully employed in the broad practice of the present invention.

Any two or more of the characteristic polymeric or oligomeric repeats can be separated by one or more amino acid residues that do not eliminate the overall phase transition characteristic of the ELP. Preferably, in fusion proteins that comprise phase transition proteins formed of polymeric or oligomeric repeats of characteristic pentapeptide Val-Pro-Gly-X-Gly, the ratio of Val-Pro-Gly-X-Gly pentapeptide units to other amino acid residues of the ELP is greater than about 75%, more preferably greater than about 85%, still more preferably greater than about 95%.

The phase transition of the FP is preferably mediated by one or more mechanisms selected from the group comprising: changing temperature; changing pH; addition of (organic or inorganic) solutes and/or solvents; side-chain ionization or chemical modification; irradiation with electromagnetic waves (rf, ultrasound, and light) and changing pressure. The preferred mechanisms for mediating the phase transition are raising temperature and adding solutes and/or solvents.

The FPs of the present invention may optionally comprise spacer sequence(s) separating the one or more biological molecules from the one or more phase transition proteins. The spacer sequence, when present, preferably comprises a cleavage site, e.g., a proteolytic cleavage site, a chemical cleavage site, a photolytic cleavage site, a thermolytic cleavage site, or a cleavage site susceptible to cleavage in the presence of a shear force, pH change, enzymatic agent, ultrasonic or other predetermined frequency field providing energy effective for cleavage. The cleavage modality may be of any of widely varying types, it being necessary only that the cleaving step yield at least one biological molecule (as a cleavage product) that retains functional utility for its intended purpose.

The FPs of the present invention may also optionally comprise signal peptides for directing secretion of the FPs from the cell, so that the FPs may readily be isolated from the medium of an active culture of recombinant cells genetically modified to produce the FPs. Such signal peptides are preferably cleavable from the fusion protein by enzymatic cleavage.

Such FPs may be synthetically, e.g., recombinantly, produced.

In a preferred aspect, the invention provides a fusion protein exhibiting a phase transition, the fusion protein comprising: (a) one or more protein(s) of interest; (b) one or more protein(s) exhibiting a phase transition joined at a C- and/or N-terminus of a protein of (a); and (c) optionally, a spacer sequence separating the any of the protein(s) of (a) and/or (b).

In another preferred aspect, the invention provides a fusion protein exhibiting a phase transition, said fusion protein comprising: (a) one or more proteins of interest; (b) one or more α-turn protein(s) joined at a C- and/or N-terminus of any of the proteins of (a); and (c) optionally, a spacer sequence separating any of the protein(s) of (a) and/or (b).

In yet another preferred aspect, the invention provides a fusion protein exhibiting a phase transition, the fusion protein comprising: (a) a protein of interest; (b) a protein exhibiting a phase transition joined at a C- and/or N-terminus of the protein of interest; and (c) optionally, a spacer sequence separating the protein or peptide of (a) from the protein of (c).

In another preferred aspect, the invention provides a fusion protein exhibiting a phase transition, said fusion protein comprising: (a) a protein of interest; (b) a protein exhibiting a β-turn joined at a C- and/or N-terminus of the protein of (a); and (c) optionally, a spacer sequence separating the protein of (a) from the protein of (c).

In a related aspect, the invention provides a polynucleotide comprising a nucleotide sequence encoding a fusion protein exhibiting a phase transition, said fusion protein comprising: (a) one or more proteins of interest; (b) one or more proteins, e.g., β-turn proteins, exhibiting a phase transition joined at a C- and/or N-terminus of (a); and (c) optionally, a spacer sequence separating any of the protein(s) of (a) and/or (b). The polynucleotide may be provided as a component of an expression vector. The invention also provides a host cell (prokaryotic or eukaryotic) transformed by such expression vector to express the fusion protein.

In a related aspect, the invention provides a method of producing one or more fusion proteins comprising: (a) transforming a host cell with the expression vector; and (b) causing the host cell to express the fusion protein. In a preferred aspect, the fusion protein comprises a signal sequence directing secretion of the fusion protein from the cell so that the fusion protein may be isolated and/or partially purified from the culture medium.

The invention also provides a method for isolating and/or partially purifying one or more fusion proteins comprising: (a) expressing the fusion protein(s) by host cells as described in the preceding paragraph; (b) causing the cells to release the fusion protein, e.g., by secretory release from such cells, or by disrupting the cells to release the fusion proteins, as for example by use of a lytic agent, sonication conditions, etc.; and (c) isolating and/or partially purifying the proteins by a method comprising effecting a phase transition, e.g., by raising temperature of the fusion protein in a solvating medium containing the fusion protein, or in other manner as more fully described elsewhere herein.

In a preferred mode, the invention provides a method for isolating and/or partially purifying one or more fusion proteins from a culture comprising cells expressing such fusion proteins, the method comprising: (a) expressing the fusion proteins; (b) isolating the fusion proteins by a method which comprises effecting a phase transition, e.g., by raising temperature or other manner manifesting a phase transition of the fusion protein.

The invention further provides a method of optimizing size of an ELP expression tag incorporated in a polynucleotide comprising a nucleotide sequence encoding a fusion protein exhibiting a phase transition, wherein the fusion protein comprises a protein of interest. Such method comprises the steps of (i) forming a multiplicity of polynucleotides comprising a nucleotide sequence encoding a fusion protein exhibiting a phase transition, wherein each of such multiplicity of polynucleotides includes a different-sized ELP expression tag, (ii) expressing corresponding fusion proteins from such multiplicity of polynucleotides, (iii) determining a yield of the desired protein for each of the corresponding fusion proteins, (iv) determining size of particulates for each of the corresponding fusion proteins in solution as temperature is raised above Tₜ, and (v) selecting an optimized size ELP expression tag according to predetermined selection criteria, e.g., for maximum recoverable protein of interest from among said multiplicity of polynucleotides, or for achieving a desired balance between yield and ease of isolation ability for each of the proteins of interest produced from the respective polynucleotides.

The invention relates in another aspect to an ELP fusion protein comprising an optimized ELP tag, produced as a product of the aforementioned optimization method.

The ITC purification technique of the invention can be scaled down and multiplexed for concurrent, parallel laboratory scale purification from numerous cell cultures, to achieve simultaneous purification of proteins from multiple cultures. Such high-throughput purification application of the invention can be utilized, for example, to expedite both structure-function studies of proteins and the screening of proteins in pharmaceutical studies.

The invention provides in a further aspect a method of purification of fusion proteins to yield a protein of interest, by steps including forming a polynucleotide comprising a nucleotide sequence encoding a fusion protein exhibiting a phase transition, expressing the fusion protein in culture, and subjecting a fusion protein-containing material from the culture to processing involving separation (e.g., by centrifugation, membrane separation, etc.) and inverse transition cycling to recover the protein of interest. In such methodology, the fusion protein-containing material from the culture may be the culture itself, or a subsequent processing fraction derived from the culture such as a lysed cellular suspension, cell pellets, supernatants, etc. The respective steps may be carried out on one or more microplates, as part of a high throughput purification arrangement for practicing the ITC method of the invention.

Another aspect of the invention relates to a method of purifying a protein of interest from a medium containing same, comprising adding to said medium an ELP-tagged purification agent that interacts with the protein of interest to form a complex therewith, subjecting said medium containing said complex to ITC to insolubilize and aggregate the complex, and recovering the aggregated complex that comprises the protein of interest from said medium.

A further aspect of the invention relates to a method of producing a purified protein of interest, comprising:
providing a fusion protein comprising the protein of interest and an ELP tag, wherein the fusion protein contains at least one cleavage site that is cleavable to yield the protein of interest as a cleavage product;
contacting the fusion protein with an ELP-tagged cleavage agent that is effective to cleave said cleavage site, thereby yielding said protein of interest as a cleavage product, in a cleavage product mixture comprising said ELP tag, any uncleaved fusion protein, and said ELP-tagged cleavage agent;
subjecting the cleavage product mixture to ITC to insolubilize and aggregate each of said ELP tag, any uncleaved fusion protein and ELP-tagged cleavage agent; and
recovering the protein of interest.

The cleavable ELP fusion proteins of the invention may in various embodiments comprise multiple cleavage sites. Such multiple cleavage site fusion proteins may be usefully employed to sequentially fractionate the fusion protein into portions of interest, e.g., by corresponding sequential ITC steps, so that the protein of interest as such term is used herein may actually comprise multiple constituent protein components, e.g., two or more protein products.

In a further aspect, the invention relates to a method of production of a protein of interest, comprising expressing the protein of interest in a culture medium, binding the expressed protein of interest to an ELP tag, and recovering the expressed protein of interest bound to the ELP tag by a recovery process comprising ITC.

Yet another aspect of the invention relates to a method of automated high-throughput protein purification, comprising
providing a multi-well filter block,
introducing to wells of the multi-well filter block transformed cells expressing fusion proteins including a protein of interest and an ELP tag,
incubating said cells to express said fusion proteins,
lysing said cells in said wells,
heating the multi-well filter block to precipitate said fusion proteins, and
removing cell debris from said fusion proteins.

A further aspect of the invention relates to a method of protein production in which a protein of interest is produced as a component of an ELP fusion protein and said ELP fusion protein is subjected to ITC for recovery thereof under ITC conditions effective therefor, comprising monitoring recovery of said ELP fusion protein, and responsively adjusting said ITC conditions to maintain a predetermined level of said recovery of said ELP fusion protein.

Additional aspects of the invention variously relate to:
an ELP fusion protein containing a cleavage site that is non-proteolytically cleavable;
an ELP fusion protein containing a photolabile cleavage site;
an ELP fusion protein containing a thermally labile cleavage site;
an ELP fusion protein containing a cleavage site cleavable by exposure to light or other electromagnetic radiation, change of pH, or change of temperature;
an ELP fusion protein comprising an ELP moiety including polymeric or oligomeric repeats of a polypeptide selected from the group consisting of VPGG, IPGG, AVGVP, IPGVG, LPGVG, VAPGVG, GVGVPGVG, VPGFGVGAG, and VPGVGVPGG;
an ELP fusion protein comprising a signal peptide sequence;
an ELP fusion protein comprising a heat shock protein sequence;
a thermophilic prokaryotic cell transformed to express an ELP fusion protein;
a mesophilic prokaryotic cell transformed to express an ELP fusion protein.
a thermotolerant prokaryotic cell transformed to express an ELP fusion protein;
an eukaryotic cell transformed to express an ELP fusion protein; and
a thermotolerant prokaryotic cell transformed to express an ELP fusion protein, wherein the ELP fusion protein comprises an ELP moiety and a protein of interest, and a cleavage moiety including a thermally labile bond cleavable at a temperature above temperature of ITC phase transition of the ELP fusion protein.

An additional aspect of the invention relates to a method of protein production, comprising expressing in an expression medium an ELP fusion protein including a protein of interest, recovering the ELP fusion protein from the expression medium by a recovery process including thermally-mediated ITC, and subjecting the recovered ELP fusion protein to a non-enzymatic separation of the protein of interest from the ELP fusion protein.

The invention in one aspect contemplates an ELP fusion protein including an ELP moiety and a protein of interest, wherein the ELP fusion protein comprises a cleavage moiety between the ELP moiety and the protein of interest, and the cleavage moiety includes a cleavage site that is cleavable by a modality selected from the group consisting of thermolysis, photolysis, shear-mediated lysis, pH change, and exposure to an ultrasonic or predetermined frequency field providing energy effective for cleavage.

Additional aspects of the invention relate to prokaryotic cells transformed to express an ELP fusion protein, as well as eukaryotic cells transformed to express an ELP fusion protein.

A further aspect of the invention relates to an ELP fusion protein including an ELP moiety comprising polymeric or oligomeric repeat units of a polypeptide selected from the group consisting of VPGG, IPGG, AVGVP, IPGVG, LPGVG, VAPGVG, GVGVPGVG, VPGFGVGAG, VPGVGVPGG, and combinations thereof.

Another ELP fusion protein in accordance with the invention includes an ELP moiety comprising polymeric or oligomeric repeat units selected from the group consisting of LPGXG (SEQ ID NO: 11), IPGXG (SEQ ID NO: 12), and combinations thereof, wherein X is an amino acid residue that does not preclude phase transition of the ELP fusion protein.

In another method aspect, the invention relates to a protein production method, comprising:
providing cells in culture, wherein said cells have been transformed to express an ELP fusion protein including a thermally labile bond between an ELP moiety and a protein of interest in said ELP fusion protein;
incubating the cells to express said ELP fusion protein;
releasing said ELP fusion protein from said cells;
subjecting the ELP fusion protein to a purification process including ITC processing at a first elevated temperature;
heating the ELP fusion protein from the purification process to temperature above said first elevated temperature to thermally break the thermally labile bond, and yield said ELP moiety and said protein of interest as thermolysis products; and
subjecting said thermolysis products to ITC processing to recover said protein of interest.

Additional methodology of the invention relates to a method of protein production including culturing transformed cells for expression of secretory ELP fusion proteins and secretion of ELP fusion proteins from the cells, and subjecting the secreted ELP fusion proteins to ITC at elevated temperature for purification thereof, comprising inducing heat shock protein production in the cells.

A still further aspect of the invention relates to a method of producing a protein of interest including subjecting an ELP fusion protein comprising the protein of interest, to ITC for recovery of the ELP fusion protein, wherein said ITC effects aggregation of desolubilized particles of the ELP fusion protein, comprising monitoring size of aggregates of the desolubilized particles of the ELP fusion protein, and responsively adjusting temperature so that said aggregates are maintained in an aggregate size regime to achieve a predetermined yield of the protein of interest.

In another method aspect, the invention relates to a method of protein production including recovery of ELP fusion protein material from a medium containing same by a recovery process including ITC, wherein said ELP fusion protein material comprises a population of ELP fusion proteins having ELP tags of different lengths, in mixture with one another, thereby maintaining stable yields, separability and aggregate size of the ELP fusion protein material, whereby perturbations of temperature or other environmental conditions do not cause gross deviations in the level of recovery of the purified protein of interest.

A further method of protein purification according to the invention comprises expressing a fusion protein including a protein of interest and an affinity tag, and contacting the fusion protein, in a medium containing same, with an ELP-protein whose protein moiety binds to said affinity tag, thereby forming a protein complex comprising said fusion protein and ELP-protein, and subjecting the protein complex to ITC to recover same from said medium.

Yet another method aspect of the invention relates to a method of protein production including expression of an ELP fusion protein including a protein of interest and a cleavage site that is enzymatically cleavable to release the protein of interest from the ELP fusion protein, such method comprising
subjecting the ELP fusion protein to ITC for purification thereof,
contacting the purified ELP fusion protein with an ELP-tagged enzyme effective for enzymatically cleaving ELP fusion protein to release the protein of interest from the ELP fusion protein and produce a cleavage mixture including the protein of interest, ELP, uncleaved fusion protein, and the ELP-tagged enzyme,
subjecting the cleavage mixture to ITC to insolubilize ELP, uncleaved fusion protein, and the ELP-tagged enzyme, and
recovering the protein of interest from the cleavage mixture.

A still other method aspect of the invention relates to a method of protein production including expression of an ELP fusion protein including a protein of interest and an acid-cleavable-Asp-Pro-cleavage site that is acid-cleavable to release the protein of interest from the ELP fusion protein, such method comprising:
subjecting the ELP fusion protein to ITC for purification thereof,
contacting the purified ELP fusion protein with acid that is effective for cleaving the ELP fusion protein to release the protein of interest from the ELP fusion protein and produce a cleavage mixture including the protein of interest, ELP, and uncleaved fusion protein,,
subjecting the cleavage mixture to ITC to insolubilize ELP and uncleaved fusion protein, and
recovering the protein of interest from the cleavage mixture.

In a further aspect, the invention relates to a method for producing a fusion protein including a therapeutic protein and an ELP tag, comprising:
(i) expressing the fusion protein in a transformed host cell;
(ii) secreting the fusion protein from the host cells, or alternatively disrupting the host cells to release the fusion protein;
(iii) aggregating the fusion protein by a method that comprises ITC;
(iv) concentrating the aggregated fusion protein by centrifugation;
(v) discarding the supernatant and resolubilizing the pelleted fusion protein;
(vi) adding an enzyme to cleave the therapeutic protein from its ELP-tag;
(vii) aggregating free ELP-tag by a method that comprises ITC;
(viii) concentrating the aggregated free ELP-tag by centrifugation; and
(ix) recovering supernatant containing the therapeutic protein.

In still a further aspect, the present invention relates to a method of conducting a biocatalytic reaction in a reaction zone, comprising utilizing a biocatalyst to catalyze the reaction, wherein the biocatalyst comprises an ELP fusion protein, and removing the biocatalyst from the reaction zone by ITC.

Various other aspects, features and embodiments of the invention will be more fully apparent from the ensuing disclosure and appended claims.

### Definitions

The word “transform” is broadly used herein to refer to introduction of an exogenous polynucleotide sequence into a prokaryotic or eukaryotic cell by any means known in the art (including, for example, direct transmission of a polynucleotide sequence from a cell or virus particle as well as transmission by infective virus particles), resulting in a permanent or temporary alteration of genotype in an immortal or non-immortal cell line.

The term “protein” is used herein in a generic sense to include polypeptides of any length. The term “peptide” is used herein to refer to shorter polypeptides having from about 2 to about 100 amino acid residues.

The term “functional equivalent” is used herein to refer to a protein that is an active analog, derivative, fragment, truncation isoform or the like of a native protein. A polypeptide is active when it retains some or all of the biological activity of the corresponding native polypeptide.

As used herein, “pharmaceutically acceptable” component (such as a salt, carrier, excipient or diluent) of a formulation according to the present invention is a component which (1) is compatible with the other ingredients of the formulation in that it can be combined with the FPs of the present invention without eliminating the biological activity of the FPs; and (2) is suitable for use with animals (including humans) without undue adverse side effects (such as toxicity, irritation, and allergic response). Side effects are “undue” when their risk outweighs the benefit provided by the pharmaceutical composition. Examples of pharmaceutically acceptable components include, without limitation, any of the standard pharmaceutical carriers such as phosphate buffered saline solutions, water, emulsions such as oil/water emulsions, microemulsions and various types of wetting agents.

As used herein, the term “native” used in reference to a protein indicates that the protein has the amino acid sequence of the corresponding protein as found in nature.

### BRIEF DESCRIPTION OF THE DRAWINGS

FIG. 1 shows an inverse transition cycling purification scheme, in which a target protein fused to an ELP sequence is separated from other contaminating proteins by inducing the ELP inverse phase transition.

FIG. 2 is a schematic representation of the thioredoxin-ELP fusion protein showing the location of the thrombin cleavage site.

FIG. 3 is a schematic representation of a thioredoxin-ELP-tendamistat fusion protein showing the location of thrombin cleavage sites, one being between thioredoxin and the ELP, and the other being between the ELP and tendamistat.

FIG. 4 is a plot showing the inverse transition characterization of free ELP (thrombin-cleaved and purified from thioredoxin-ELP) (♦); thioredoxin-ELP (▴); thioredoxin-ELP-tendamistat (◯); ELP-tendamistat (cleaved and purified from thioredoxin-ELP-tendamistat) (⋄); and thioredoxin-ELP (cleaved and purified from thioredoxin-ELP-tendamistat) (□). All fusion proteins contained the same 90-mer ELP sequence, which comprises 90 repeating units of a monomeric pentapeptide. Profiles were obtained with protein concentrations of 25 μM in PBS using a 1.5° C. min⁻¹ heating rate.

FIG. 5 is a plot showing transition temperature (Tₜ), defined as 50% maximal turbidity, as a function of molecular weight (MW) in kilodaltons (kDa) for thioredoxin-FPs.

FIG. 6 is a plot of transition temperature as a function of NaCl molar concentration for the thioredoxin/60-mer FP (25 μM) in 50 mM phosphate buffer, pH 8.0.

FIG. 7 is a graph of thioredoxin activity through 3 rounds of inverse transition cycling for the thioredoxin/60-mer fusion protein, wherein an increase in temperature resulted in aggregation of the fusion protein (monitored spectrophotometrically), reduction of temperature below Tₜ caused the protein to disaggregate and the solution to clear, and thioredoxin activity, assayed after each cycle, was unaffected by the inverse transition cycling.

FIG. 8 is an SDS-PAGE characterization of inverse transition purification, showing each stage of purification for the thioredoxin/90-mer ELP fusion (49.9 kDa, lanes 1 through 5) and the thioredoxin/90-mer ELP/tendamistat (57.4 kDa, lanes 7 through 9): lanes 1 & 7, soluble lysate; lanes 2 & 8, discarded supernatant containing contaminating E. coli proteins; lanes 3 & 9: resolubilized pellet fraction containing purified fusion protein; lane 4, second round supernatant; lane 5: second round pellet; lanes 6 and 10: molecular weight markers (kDa).

FIG. 9 is a graph of total protein and thioredoxin activity for each stage of purification of the thioredoxin/90-mer ELP, wherein values were normalized to those determined for the soluble lysate.

FIG. 10 shows DNA and corresponding amino acid sequences for a 10-mer ELP gene.

FIG. 11 shows the modified pET-32b vector for production of thioredoxin-ELP fusions.

FIG. 12 shows the modified pET-32a vectors for the production of the thioredoxin-ELP-tendamistat fusion with alternate thrombin recognition sites.

FIG. 13 is a graph of optical density at 350 nm as a function of temperature for solutions of the thioredoxin-ELP fusion proteins.

FIG. 14 is a graph showing the heating and cooling turbidity profiles for the solution conditions used in ITC purification, for solutions of thioredoxin-ELP1 [V-20] (solid lines) and thioredoxin-ELP1 [V₅A₂G₃-90] (dashed lines) at lysate protein concentrations in PBS with 1.3 M NaCl.

FIGS. 15-20 illlustrate the effect of temperature on the particle size distribution of ELP1 [V₅A₂G₃-90] in PBS (FIGS. 15 and 16), PBS+1 M NaCl (FIGS. 17 and 18), and PBS+2 M NaCl (FIGS. 19 and 20). FIGS. 15, 17 and 19 show the effect of temperature on particle sizes of monomers (diamonds) and aggregates (squares). Analysis artifacts (stars) and network contributions (triangles), which may result from the coordinated slow movements of a network of smaller particles, are also shown (see text for discussion). FIGS. 16, 18 and 20 show the percentage of the scattered intensity attributed to each type of particle as a function of temperature.

FIGS. 21-24 show the effect of temperature on the particle size distribution of ELP[V-20] in PBS+1 M NaCl (FIGS. 21 and 22) and PBS+2 M NaCl (FIGS. 23 and 24). FIGS. 21 and 23 show the effect of temperature on particle sizes of monomers (diamonds), 12 nm particles (circles), and larger aggregates (squares). Network contributions are also shown (triangles). FIGS. 22 and 24 show the percentage of the scattered intensity attributed to each type of particle as a function of temperature.

FIG. 25 shows SDS-PAGE analysis of ITC purification. Lane A shows a molecular weight marker, labeled in kDa. Lanes B-D show IMAC purification of free thioredoxin(His₆), and Lanes E-H and I-L show ITC purification of thioredoxin-ELP1 [V-20] and thioredoxin-ELP1 [V₅A₂G₃-90], respectively. Lanes B, E, and I are the soluble cell lysate. Lanes C and D are the IMAC column flow-through and elution product, respectively. For ITC purification, lanes F and J are the supernatant after inverse transition and centrifugation; lanes G and K are the pellet containing the target protein, after redissolving in PBS; and lanes H and L are the purified target protein thioredoxin, after cleavage with thrombin and separation from its ELP tag by a second round of ITC.

FIG. 26 is a graph of purified protein yield. The total yields of the thioredoxin(His₆), thioredoxin-ELP1 [V-20], and thioredoxin-ELP1 [V₅A₂G₃-90] from the 50 ml test cultures are shown, extrapolated to milligrams per liter of culture (mean±SD, n=4). The separate contributions of the ELP tag and thioredoxin to the yield, as calculated using their respective mass fractions of the fusion protein, are also shown for comparison.

FIG. 27 shows SDS-PAGE analysis of the effect of NaCl concentration and centrifugation temperature on purification of thioredoxin-ELP[V-20] by ITC: SL=soluble cell lysate; S=supernatant after inverse transition of fusion protein and centrifugation to remove aggregated target protein; and P=redissolved pellet containing the purified fusion protein, after dissolution in PBS. The molar NaCl concentration and centrifugation temperature for each purification is noted at top.

FIG. 28 is an SDS-PAGE gel of the stages of high throughput protein purification using microplates and inverse transition cycling according to the above-described procedure, in which ELP/thioredoxin fusion protein was purified (Lane 1: molecular mass markers (kDa) (Sigma, wideband; Lane 2: crude lysate; Lane 3: insoluble proteins; Lane 4: soluble lysate; Lane 5: supernatant containing contaminant proteins; Lane 6: purified ELP/thioredoxin fusion protein; and Lanes 7 and 8: purified ELP/thioredoxin fusion proteins from other wells).

FIG. 29 is a histogram of total fusion protein per well as determined using absorbance measurements (A₂₈₀, ε=19,870) (n=20, μ=32.97, σ=8.48).

FIG. 30 is a histogram of fusion protein functionality/purity for each sample compared to commercial thioredoxin (from Sigma) (n=20, μ=110.37%, σ=16.54%).

FIG. 31 shows SDS-PAGE analysis for ELP1-20/thioredoxin protein purified from cell cultures in microplates by ITC (Lane A: molecular mass markers (kDa); Lane B: cell extract; Lane C: insoluble protein; Lane D: soluble lysate; Lane E: supernatant containing contaminant proteins; and Lanes F, G and H: ITC purified ELP1-20/thioredoxin).

### DETAILED DESCRIPTION OF THE INVENTION

The disclosure of priority U.S. patent application Ser. No. 09/812,382 is hereby incorporated herein by reference in its entirety for all purposes.

The invention generally provides a fusion protein (FP) exhibiting a phase transition, the fusion protein comprising: (a) one or more biological molecules; (b) one or more proteins exhibiting a phase transition joined to the biologically active molecule(s); and (c) optionally, a spacer sequence separating any of the protein(s) of (b) from any of the biological molecule(s) of (a). The phase transition component of the FPs is preferably an ELP as described herein.

The invention also relates to methods of isolating and/or partially purifying the FPs and optionally, further cleaving and isolating the biological molecule component of the. FPs, as well as high-throughput purification applications of the methodology of the invention.

Protein or Peptide with Phase Transition Characteristics

The FPs of the invention comprise an amino acid sequence endowing the FP with phase transition characteristics.

The phase transition component of the FP may comprise a β-turn component. The β-turn component is suitably derived from pentapeptide repeats found in mammalian elastin, such as elastin-like peptides (ELPs). Examples of polypeptides suitable for use as the β-turn component are described in Urry, et al. International Patent Application PCT/US96/05186. Alternatively, the phase transition component of the FP can be a component lacking a β-turn component, or otherwise having a different conformation and/or folding character.

The ELPs may comprise polymeric or oligomeric repeats of various tetra-, penta-, hexa-, hepta-, octa-, and nonapeptides, including but not limited to VPGG, IPGG, VPGXG, AVGVP, IPGVG, LPGVG, VAPGVG, GVGVPGVG, VPGFGVGAG, and VPGVGVPGG (SEQ NO: 1 to SEQ NO: 10). It will be appreciated by those of skill in the art that the ELPs need not consist of only polymeric or oligomeric sequences as listed hereinabove, in order to exhibit the desired phase transition, and that other polymeric or oligomeric sequences of varying size and constitution that exhibit phase transition behavior are also usefully employed in the broad practice of the present invention.

Preferably, such ELPs are polymeric or oligomeric repeats of the pentapeptide VPGXG (SEQ ID NO: 3), where the guest residue X is any amino acid that does not eliminate the phase transition characteristics of the ELP. X may be a naturally occurring or non-naturally occurring amino acid. For example, X may be selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine. In one aspect of the invention X is not proline.

X may be a non-classical amino acid. Examples of non-classical amino acids include: D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

Alternatively, such ELPs can be polymeric or oligomeric repeats of the pentapeptide IPGXG (SEQ ID NO: 11) or LPGXG (SEQ ID NO: 12), where X is as defined hereinabove.

The polymeric or oligomeric repeats of the ELP sequences may be separated by one or more amino acid residues that do not eliminate the overall phase transition characteristic of the FPs. In a preferred aspect of the invention, when the ELP component of the fusion protein comprising polymeric or oligomeric repeats of the pentapeptide VPGXG, the ratio of VPGXG repeats to other amino acid residues of the ELP is greater than about 75%, more preferably greater than about 85%, still more preferably greater than about 95%, and most preferably greater than about 99%.

Different ELP constructs are distinguished here using the notation ELPk [XᵢYⱼ-n], where k designates the specific type of ELP repeat unit, the bracketed capital letters are single letter amino acid codes and their corresponding subscripts designate the relative ratio of each guest residue X in the repeat units, and n describes the total length of the ELP in number of the pentapeptide repeats. For example, ELP1 [V₅A₂G₃-10] designates a polypeptide containing 10 repeating units of the pentapeptide VPGXG, where X is valine, alanine, and glycine at a relative ratio of 5:2:3; ELP1 [K₁V₂F₁-4] designates a polypeptide containing 4 repeating units of the pentapeptide VPGXG, where X is lysine, valine, and phenylalanine at a relative ratio of 1:2:1; ELP1 [K₁V₇F₁-9] designates a polypeptide containing 4 repeating units of the pentapeptide VPGXG, where X is lysine, valine, and phenylalanine at a relative ratio of 1:7:1; ELP1 [V-5] designates a polypeptide containing 5 repeating units of the pentapeptide VPGXG, where X is exclusively valine; ELP1 [V-20] designates a polypeptide containing 20 repeating units of the pentapeptide VPGXG, where X is exclusively valine; ELP2 [5] designates a polypeptide containing 5 repeating units of the pentapeptide AVGVP; ELP3 [V-5] designates a polypeptide containing 5 repeating units of the pentapeptide IPGXG, where X is exclusively valine; ELP4 [V-5] designates a polypeptide containing 5 repeating units of the pentapeptide LPGXG, where X is exclusively valine.

Preferred ELPs are those that provide the FP with a transition temperature (Tₜ) that is within a range that permits the FP to remain soluble while being produced in a recombinant organism. It will be understood by one of skill in the art that the preferred Tₜ will vary among organisms in respect of their temperature requirements for growth. For example, where the microbe used to culture the FP is E. coli, the preferred Tₜ is from about 37.5 to about 42.5° C. in water, preferably about 40° C. in water. Useful and preferred temperatures can be readily determined by one of skill in the art for any organism on the basis of the description herein.

Preferred transition temperatures are those that permit solubility in the recombinant organism during culturing and permit aggregation of the FP by a small increase in temperature following cell lysis. For example, a preferred difference between the culture temperature and the Tₜ is in the range of about 30 to about 40° C. In another aspect, the temperature increase is in the range of about 1 to about 7.5° C.; more preferably, the required temperature increase is in the range of about 1 to about 5° C.

It will be understood that the foregoing relatively narrow temperature ranges utilized for induction of phase transition of the fusion protein may be relaxed by the use of thermotolerant organisms and cells, e.g., thermophilic and mesophilic bacteria, in the cell culture in which the fusion protein is being expressed.

Further, the fusion protein may employ a thermally labile bond between the protein of interest and the phase transition-conferring component of the fusion protein, to permit elevation of temperature to be employed both as an induction modality for phase transition of the fusion protein (at a first elevated temperature) and (in further elevation to a second elevated temperature higher than the first elevated temperature) as a modality for cleaving the thermally labile bond to yield the phase transition-conferring component of the fusion protein and the protein of interest.

The FP in one aspect comprises a signal peptide to direct secretion of the fusion protein from the thermotolerant cells in culture, with the culture disposed on one face of a membrane that is permselective for the fusion protein, and with fusion protein permeate thus separated from the culture being flowed through a first downstream “hot zone” for ITC processing and purification of the fusion protein, followed by processing of the fusion protein in a second downstream “hot zone” for cleavage of the thermally labile bond to yield the protein of interest and the phase-transition-conferring component of the fusion protein, as cleavage products. Subsequent ITC processing then is employed recover the protein of interest from the cleavage products mixture containing same.

It will be appreciated that the foregoing process may be arranged with respective process streams in heat exchange relationship with each other, to permit sensible heat to be recovered from hot process streams and transferred to streams to be heated in the operation of the process, thereby maximizing the efficiency of the overall process.

The invention in a further aspect utilizes heat shock proteins in the culturing cells to moderate adverse effects of temperatures required for inducing phase transition of secreted fusion proteins in the culture medium, as part of a continuous process. Heat shock protein expression may be induced by hyperthermalizing the cultured cells in a take-off stream (side stream) from a bioreactor tank containing the cell culture, or by modifying the cultured cells to overexpress heat shock proteins during residence of the cultured cells in the bioreactor.

Previous studies by Urry and colleagues have shown that the fourth residue (X) in the elastin pentapeptide sequence, VPGXG, can be altered without eliminating the formation of the β-turn. These studies also showed that the Tₜ is a function of the hydrophobicity of the guest residue. By varying the identity of the guest residue(s) and their mole fraction(s), ELPs can be synthesized that exhibit an inverse transition over a 0-100° C range.

The Tₜ at a given ELP length can be decreased by incorporating a larger fraction of hydrophobic guest residues in the ELP sequence. Examples of suitable hydrophobic guest residues include valine, leucine, isoleucine, phenyalanine, tryptophan and methionine. Tyrosine, which is moderately hydrophobic, may also be used. Conversely, the Tₜ can be increased by incorporating residues, such as those selected from the group consisting of: glutamic acid, cysteine, lysine, aspartate, alanine, asparagine, serine, threonine, glysine, arginine, and glutamine; preferably selected from alanine, serine, threonine and glutamic acid.

The ELP is preferably selected to provide the FP a Tₜ ranging from about 10 to about 80° C., more preferably from about 35 to about 60° C., most preferably from about 38 to about 45° C. However, as stated above, the preferred Tₜ varies with the required culture conditions of the organism in which the FP will be cultured.

The Tₜ can also be varied by varying ELP chain length. By way of specific illustrative example, the Tₜ's of the higher molecular weight ELPs are in the vicinity of 42° C. for the thioredoxin/180-mer fusion (at 25 μM in PBS). The Tₜ increased dramatically with decreasing MW. In low ionic strength buffers, the Tₜ's of the lower molecular weight ELPs are often too high for protein purification, absent the use of thermophils, mesophils, or other thermotolerant cellular species, and/or heat shock protein expression, as previously discussed. In such cases, a high concentration of NaCl or other ionic solute, or other organic or inorganic solute or solvent species, can be used to decrease the Tₜ to a useful temperature.

For polypeptides having a molecular weight >100,000, the hydrophobicity scale developed by Urry et al. (PCT/US96/05186) is preferred for predicting the approximate Tₜ of a specific ELP sequence.

For polypeptides having a molecular weight <100,000, the Tₜ is preferably determined by the following quadratic function:
Tₜ=M₀+M₁X+M₂X²
where X is the MW of the FP, and M₀=116.21; M₁=−1.7499; M₂=0.010349.

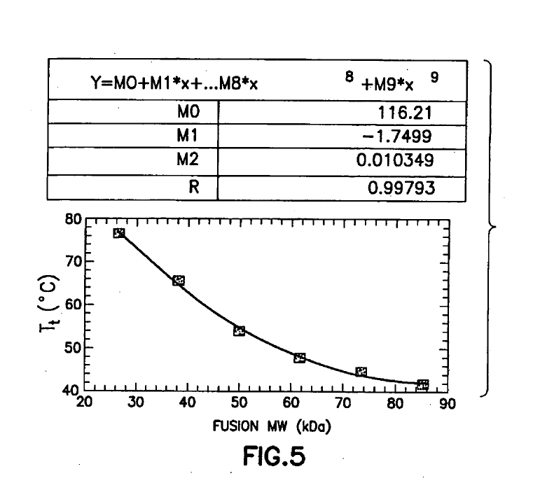

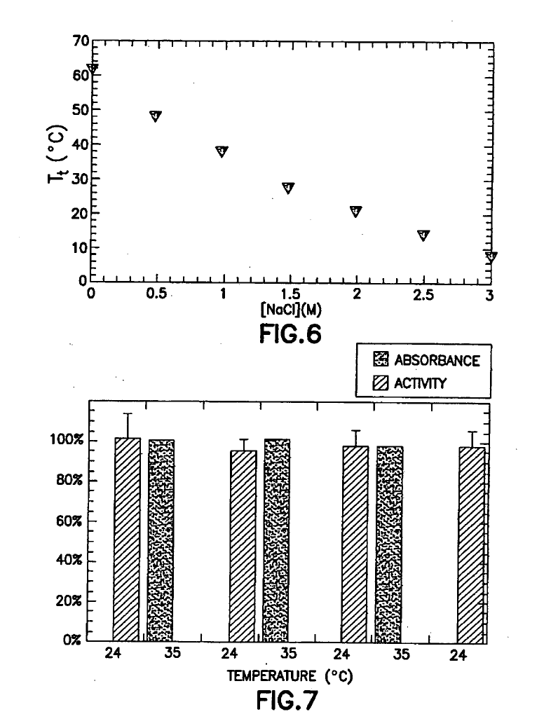

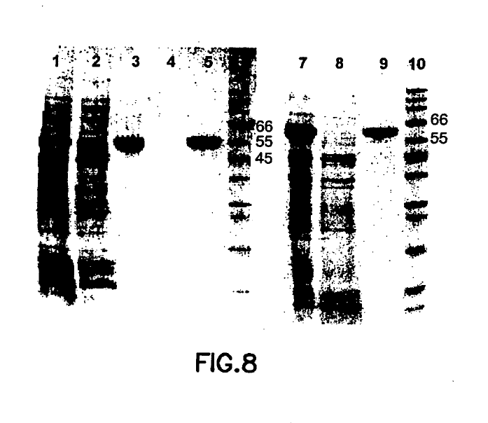

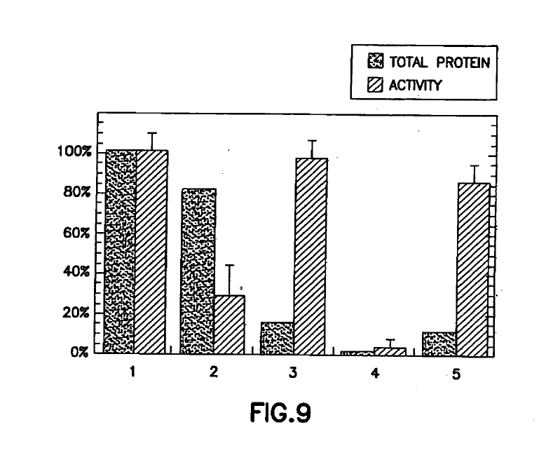

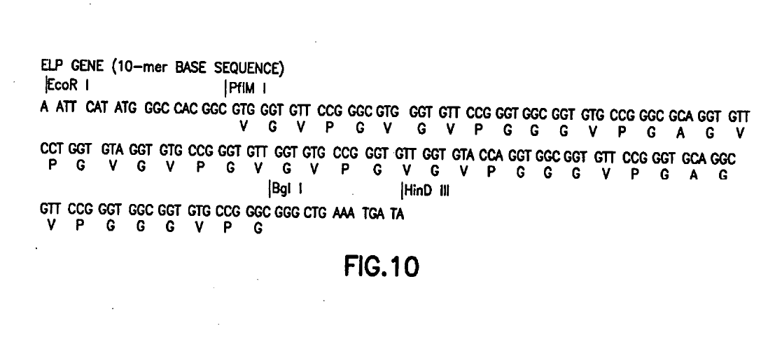

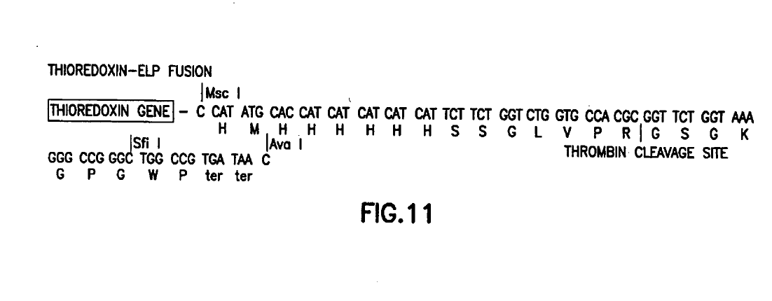

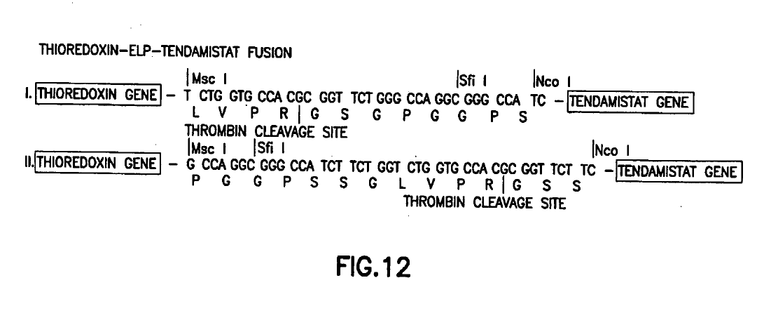

The regression coefficient for this fit is 0.99793 (see FIG. 5, discussed more fully hereinafter).

ELP chain length is also important with respect to protein yields. In addition to the decreased total yield of expressed fusion protein observed with increasing ELP MW, the weight percent of target protein versus the ELP also decreases as the MW of the ELP carrier increases. In a preferred aspect of the invention, the ELP length is from 5 to about 500 amino acid residues, more preferably from about 10 to about 450 amino acid residues, and still more preferably from about 15 to about 150 amino acid residues. ELP length can be reduced while maintaining a target Tₜ by incorporating a larger fraction of hydrophobic guest residues in the ELP sequence.

Reduction of the size of the ELP tag may be employed to substantially increase the yield of the target protein, as shown by the results presented hereinafter, wherein reduction of the ELP tag from 36 to 9 kDa increased the expression yield of thioredoxin by a factor of four, to a level comparable to free thioredoxin expressed without an ELP tag, while still allowing efficient and effective purification.

Truncation of the ELP tag, however, results in more complex transition behavior than observed with larger tags. In the case of thioredoxin, dynamic light scattering experiments showed that for both tags, large aggregates with hydrodynamic radii of 2 μm formed as the temperature was raised to above Tₜ. These aggregates persisted at all temperatures above the Tₜ for the thioredoxin fusion with the larger 36 kDa ELP tag. With the 9 kDa tag, however, smaller particles with hydrodynamic radii of ˜12 nm began to form at the expense of the initial larger aggregates as the temperature was raised further above the Tₜ.

Since only large aggregates can be effectively retrieved by centrifugation, efficient purification of fusion proteins with short ELP tags requires selection of solution conditions that favor the formation of the larger aggregates. Despite this additional complexity, the ELP tag can be successfully truncated to enhance the yield of a target protein without compromising purification and recovery level.

In one aspect of the present invention, the above-described susceptibility of the fusion protein to form disproportionately small, difficult-to-separate aggregates at shorter ELP tag length at temperatures above Tₜ, combined with the disproportionately higher yields achieved at shorter ELP tag length, and the desirability of keeping the temperature of the fusion protein-containing medium as close to the Tₜ of the fusion protein as possible consistent with efficient aggregate formation, is efficiently accommodated by monitoring the aggregate size being formed in the phase transition, and responsively adjusting temperature so that aggregate formation is maintained in an aggregate size regime that is consistent with good separability of the fusion protein from the FP-containing medium, and high yield of the protein of interest.

Another aspect of the present invention relates to the use of a population of fusion proteins having phase transition-endowing proteins, e.g., ELP tags, of different lengths, in mixture with one another, to maintain stable yields, separability and aggregate size, so that small perturbations of temperature or other environmental conditions do not cause gross deviations in the level of recovery of the purified protein of interest. By such provision of a heterogeneous population of differently sized ELP tags, the protein purification process is buffered against process upsets, so that the output of the protein of interest from the process is maintained at a consistent and stable level, relative to a corresponding process utilizing a homogeneous fusion protein population having same-sized ELP tags.

Yet another aspect of the invention relates to a protein purification process comprising expression of a population of fusion proteins having phase transition-endowing proteins, e.g., ELP tags, of different lengths, in mixture with one another, to maintain stable yields, separability and aggregate size, so that small perturbations of temperature or other environmental conditions do not cause gross deviations in the level of recovery of the purified protein of interest. In such process, the fusion proteins population is subjected to a phase transition to aggregate the fusion proteins, and the aggregated fusion proteins are separated from the mixture, followed by separation of the aggregated fusion proteins to recover a protein of interest therefrom. The output of the process is monitored, e.g., the level of production of the protein of interest, and the fusion proteins population is responsively adjusted to maintain the level of recovery at a predetermined level. Such adjustment may for example take the form of adding a greater or lesser proportion of one or more of differently ELP-sized sub-populations of fusion proteins so that the relative proportions of the differently ELP-sized sub-populations of fusion proteins relative to one another are balanced to achieve the continuous achievement of the desired level of production of the protein of interest.

The process variable(s) monitored in the above-described process embodiments of the invention may be any suitable variable(s), including for example, temperature of the fusion proteins mixture, turbidity, opacity, light scattering, or light attenuation of the mixture in response to impingement of a light beam on the mixture for monitoring of the concentration and size of the aggregates formed in the phase transition.

A further aspect of the invention involves use of in vitro tags for protein purification, in which protein of interest is expressed with a common affinity tag such as maltose binding protein (MBP), glutathione S-transferase (GST), biotin carboxyl carrier protein, thioredoxin, cellulose binding domain, or short peptide tags such as oligohistidine, S-peptide, and the FLAG peptide. A fusion protein containing ELPs and an affinity ligand specific for such affinity tag is added to the expression mixture to bind the protein of interest, following which ITC is conducted in accordance with the invention, to recover the protein of interest.

The invention in a still further aspect contemplates automated high throughput protein purification, in which cells engineered for fusion protein expression are loaded in a multiwell filter block, e.g., a 96-well filter block, and incubated following addition of a lysing agent. The filter block then is heated to precipitate the fusion proteins by phase transition aggregation, and cell debris is resuspended and removed in supernatent, to recover the fusion protein comprising the protein of interest.

Other high throughput protein purification methods, as well as peptide library screening processes, are contemplated by the invention, in which ELP fusion protein constructs may be employed.

In one aspect, high throughput protein purification is carried out involving a protein of interest, e.g., a therapeutic protein, which is expressed as a fusion protein from transformed cells. The fusion protein, containing a cleavage site that is enzymatically cleavable, is subjected to ITC to remove impurities, as described herein. An ELP-tagged enzyme next is added to the fusion protein to enzymatically cleave the protein of interest from the fusion protein, following which ITC is conducted to remove ELP, uncleaved fusion protein, and the ELP-tagged enzyme, thereby yielding the purified protein of interest.

Protein purification in accordance with the invention may utilize ELP-tagged external purification agents that are added to mixtures containing the protein of interest, to effect separation and purification of the protein of interest. For example, the external purification agent can be an ELP-tagged antibody or other ligand-binding protein that is specific for the protein of interest, in which target binding produces a bound entity that is separable by phase transition. Other ELP-tagged binding agents can be similarly employed.

In one aspect, the present invention relates to a method of conducting a biocatalytic reaction in a reaction zone, comprising utilizing a biocatalyst to catalyze the reaction, wherein the biocatalyst comprises an ELP fusion protein, and removing the biocatalyst from the reaction zone by ITC. The reaction zone may for example be within a bioreactor.

The ELP fusion protein for such purpose is suitably solubilized in a reaction medium in the reaction zone during the biocatalytic reaction to effect catalysis of the reaction. As one illustrative mode of operation, the ELP fusion protein is added to the reaction zone at temperature above Tₜ of the ELP fusion protein, and temperature in the reaction zone is decreased to below said Tₜ to solubilize the ELP fusion protein comprising a biocatalytic enzyme for the reaction, e.g., as a ELP-tagged biocatalyst, to effect catalysis of the reaction.

In one embodiment, cells transformed to express the ELP fusion protein are disposed in the reaction zone, and the ELP fusion protein is expressed in situ in the reaction zone from such cells, and secreted therefrom into a reaction medium in the reaction zone. The reaction medium may for example comprise an aqueous medium, e.g., as a culture medium containing the transformed cells.

Such methodology has broad application to the production of therapeutic or diagnostic agents.

Protein Component of the Fusion Protein

The FP of the invention comprises a protein of interest. The protein of interest is preferably a biologically active protein. Suitable proteins include those of interest in medicine, agriculture and other scientific and industrial fields, particularly including therapeutic proteins such as erythropoietins, inteferons, insulin, monoclonial antibodies, blood factors, colony stimulating factors, growth hormones, interleukins, growth factors, therapeutic vaccines, calcitonins, tumor necrosis factors (TNF), and enzymes. Specific examples of such therapeutic proteins include, without limitation, enzymes utilized in replacement therapy; hormones for promoting growth in animals, or cell growth in cell culture; and active proteinaceous substances used in various applications, e.g., in biotechnology or in medical diagnostics. Specific examples include, but are not limited to: superoxide dismutase, interferon, asparaginease, glutamase, arginase, arginine deaminase, adenosine deaminase ribonuclease, trypsin, chromotrypsin, papin, insulin, calcitonin, ACTH, glucagon, somatosin, somatropin, somatomedin, parathyroid hormone, erthyropoietin, hypothalamic releasing factors, prolactin, thyroid stimulating hormones, endorphins, enkephalins, and vasopressin.

In one aspect of the invention, the protein of interest is a soluble, over-expressed protein, such as thioredoxin. Thioredoxin is expressed as soluble protein at high levels in E. coli and is therefore an exemplary model for verifying that the reversible, soluble-insoluble inverse transition of the ELP tag is retained in a fusion protein. Thioredoxin also exhibits useful pharmaceutical properties and other industrially useful properties, for example, as described in U.S. Pat. Nos. 5,985,261; 5,952,034; 5,919,657; 5,792,506; 5,646,016; and 5,028,419.

In another aspect of the invention, the protein of interest is an insoluble, poorly expressed protein, such as tendamistat. Tendamistat is predominately expressed as insoluble protein in inclusion bodies. Although fusion with thioredoxin is known to promote the soluble expression of target proteins, the inventor has observed that only 5-10% of over-expressed thioredoxin-tendamistat fusion protein is recovered as soluble and functionally-active protein. It was initially expected that incorporation of a hydrophobic ELP sequence in a fusion protein that exhibits a pronounced tendency to form inclusion bodies might (1) exacerbate its irreversible aggregation in vivo during culture, and (2) cause irreversible aggregation in vitro during purification by inverse transition cycling. Surprisingly, neither problem was encountered with the ELP-tendamistat fusion protein.

The tendamistat-ELP fusion protein provides a readily-isolated, active version of tendamistat for use as an α-amylase inhibitor, e.g., in the treatment of pancreatitis. This fusion protein is suitably provided as a component of a pharmaceutical formulation in association with a pharmaceutically acceptable carrier.

Various other proteins and peptides, such as insulin A peptide, T20 peptide, interferon alpha 2B peptide, tobacco etch virus protease, small heterodimer partner orphan receptor, androgen receptor ligand binding domain, glucocorticoid receptor ligand binding domain, estrogen receptor ligand binding domain, G protein alpha Q, 1-deoxy-D-xylulose 5-phosphate reductoisomerase peptide, and G protein alpha S, have been fused with different ELP polypeptides to form FPs that exhibit inverse phase transition behavior.

The above-described proteins and peptides are significantly different in their primary, secondary, and tertiary structures, sizes, molecular weights, solubility, electric charge distribution, viscosity, and biological functions, which shows that the FPs of the present invention, when incorporating different target proteins or peptides, consistently retain the inverse phase transition behavior of the ELP tags. Therefore, the present invention has broad application in ITC-based separation and purification of various different target protein or peptide products.

The inventors have also surprisingly discovered that the protein component of the FPs retain some or all of the biological activity of the native target protein. For example, a comparison of the activity of a thioredoxin-ELP fusion protein with commercially-obtained E. coli thioredoxin showed that the thioredoxin-ELP fusion protein retains activity without requiring cleavage of the ELP tag. Similarly, tendamistat-ELP fusion protein retained most of the α-amylase inhibition activity of the free tendamistat, and after thrombin cleavage and removal of the ELP tag, tendamistat regained complete activity.

Moreover, altering solution conditions to effect isolation of the FPs did not affect the stability and activity of the FPs after transition cycling. For example, aggregation of the ELP-thioredoxin fusion above the Tₜ did not irreversibly denature the fusion protein. In fact, thioredoxin activity was completely retained after several rounds of inverse transition cycling. These results support the conclusion that desolvation and aggregation of the ELP-tagged fusion protein will not result in complete loss of activity of the protein of interest contain in such fusion protein.

Other Components of the Fusion Protein

The phase transition-imparting component of the fusion protein, e.g., an ELP having a P-turn or other conformation providing phase transition behavior, and the target protein components of the FPs of the present invention may be separated by a spacer that contains one or more cleavage sites, which can be subsequent cleaved to release the target protein components from the phase transition components of the FPs.

In one embodiment, the spacer is an amino acid sequence containing at least one cleavage site recognizable by a specific enzymatic protease. Examples include sequences cleavable by serine, cysteine (thiol), aspartyl (carboxyl) or metallo-proteases. Such protease-susceptible cleavage site permits the phase transition component of the FP to be enzymatically cleaved to enable isolation and/or partial purification of the protein of interest. Suitable enzymatic recognition sequences and cleavage sites (▾) include: -Pro-Val-▾-Gly-Pro- (Collagenase); -Asp-Asp-Asp-Lys-▾ (Enterokinase); -Ile-Glu-Gly-Arg-▾(Factor Xa); -Gly-Pro-Arg-▾(Thrombin); -Glu-Asn-Leu-Tyr-Phe-Gln-▾(Tobacco etch virus protease); -Arg-▾(Trypsin); -Arg-▾(Clostripain); and -Gly-Ala-His-Arg-▾(Ala⁶⁴-Subtilisin); Factor XIII cleavage sites and intein cleavage sites.

It will be recognized that the spacer providing a cleavage site may be of any of widely varying types, including, in addition to the enzymatically cleavable moieties just described, cleavage sites that are cleavable by exposure to light or other electromagnetic radiation, vibratory or shear forces, degradative chemical reaction (e.g., cleavage with acid or cyanogens bromide), change of pH, change of temperature, or any other means or modality for effecting scission of the spacer to yield the protein of interest and the ELP tag as scission products.

In one illustrative aspect, the spacer utilized to provide a cleavage site in the FP of the invention includes a photolabile site. An illustrative example of such a cleavage moiety is amino acid (2-nitrophenyl) glycine (Npg), an unnatural amino acid, for which a site-specific photochemical proteolysis may be employed (see England et al.(1997) Site-Specific, photochemical proteolysis applied to ion channels in vitro. Proc. Natl. Acad. Sci. USA 94: 11025-11030). Studies have shown that irradiation of proteins containing an Npg residue leads to peptide backbone cleavage at the site of the unnatural residue.

Site-specific photocleavage of hen egg lysozyme and bovine serum albumin (BSA) can be utilized as a technique for cleaving the spacer moiety, using the method described in Kumar et al. (1998) Photochemical protease: Site-Specific photocleavage of hen egg lysozyme and bovine serum albumin. Proc. Natl. Acad. Sci. USA. 95: 10,361-10,366.), in which the lysozyme is cleaved between a Trp-Val residue pair and BSA was cleaved between a Leu-Arg residue pair.

In yet another photochemical approach, vanadate may be used to effect photocleavage of phosphate binding cleavage sites of the FP. This approach takes advantage of the fact that vanadate competes for phosphate binding sites of proteins, and induces photocleavage with a high preference for serine residues, as described in Cremo et al. (1992) Biochemistry 31, 491-497; Correia et al. (1994) Arch. Biochem. Biophys. 309: 94-104.

In the general practice of the present invention involving the use of cleavable spacer moieties in the fusion protein, the use of light as a protein cleavage agent affords distinct advantages in providing precise control for the initiation and termination of photoreactions, and being environmentally benign.

In another illustrative aspect, N-(1-phenylalanine)-4-(1-pyrene) butyramide (Py-Phe), or other molecular probe, is employed to cleave a site-specific sequence of the spacer moiety.

In other aspects of the present invention, the spacer may be engineered to contain chemical cleavage sites. Chemical cleavage reagents may be employed to recognize single or paired amino acid residues and thus are useful for the release of short peptides. Chemical cleavage reagents include: cyanogen bromide, which cleaves at methionine residues (Piers et al. (1993) Gene 134: 7); N-chlorosuccinimide (Forsberg et al. (1989)Biofactors 2: 105-112) and BNPS-skatole (Knott et al. (1988) Eur. J. Biochem. 174: 405-410), which cleave at tryptophan residues, dilute acids, which cleave aspartyl-prolyl bonds (Gram et al. (1994) Biotechnology 12: 1017-1023) and hydroxylamine which cleaves asparagine-glycine bonds at basic pH (Moks et al. (1987) Bio/Technology 5: 379-382).

In a particular aspect, the technique described in U.S. Pat. No. 6,242,219 to Better and Gavit, the disclosure of which is hereby incorporated herein by reference in its entirety, is advantageously used to produce peptides from fusion proteins. In such technique, the fusion protein comprises a peptide of interest, the ELP tag and an acid-cleavable Asp-Pro site between the peptide of interest and the ELP tag. Acid treatment is used to release the peptide of interest from the fusion protein, followed by ITC separation of the ELP tag from the peptide of interest.

The FP may further be engineered to comprise a signal sequence that causes the FP to be directed to the cell surface or excreted from a recombinant organism that is used to produce the FP. The FP may be cleaved at the cell surface or may be enzymatically cleaved in solution.

The FP may also contain a sequence that permits separate purification by affinity chromatography, commonly referred to as affinity tags. Examples include His-tag, FLAG, s-tag, etc.

The FP may also contain a “detection tag,” i.e., a sequence that is retained on the protein of interest after cleavage of the phase transition component and which by virtue of binding to a reporter molecule can be used to detect the protein of interest (e.g., antibody epitopes for Western blot).

Also included within the scope of the invention are derivatives comprising FPs, which have been differentially modified during or after synthesis, e.g., by benzylation, glycosylation, acetylation, phosphorylation, amidation, PEGylation, derivatization by known protecting/blocking groups, proteolytic cleavage, linkage to an antibody molecule or other cellular ligand, etc. In one embodiment, the FPs are acetylated at the N-terminus and/or amidated at the C-terminus. In another embodiment, the FPs are conjugated to polymers, e.g., polymers known in the art to facilitate oral delivery, decrease enzymatic degradation, increase solubility of the polypeptides, or otherwise improve the chemical properties of the therapeutic polypeptides for administration to humans or other animals. The polymers may be joined to the FPs by hydrolyzable bonds. For example, in one aspect where the FPs are therapeutically active, the polymers are joined to the FPs by hydrolyzable bonds, so that the polymers are cleaved in vivo to yield the active therapeutic FPs.

Methods for Preparing the Fusion Proteins

The FPs of the invention can be obtained by known recombinant expression techniques. To recombinantly produce an FP, a nucleic acid sequence encoding the FP is operatively linked to a suitable promoter sequence such that the nucleic acid sequence encoding such FP will be transcribed and/or translated into the desired FP in the host cells. Preferred promoters are those useful for expression in E. coli, such as the T7 promoter.

Any commonly used expression system may be used, e.g., eukaryotic or prokaryotic systems. Specific examples include yeast, pichia, baculovirus, mammalian, and bacterial systems, such as E. coli, and Caulobacter.

A vector comprising the nucleic acid sequence can be introduced into a cell for expression of the FP. The vector can remain episomal or become chromosomally integrated, as long as the gene carried by it can be transcribed to produce the desired RNA. Vectors can be constructed by standard recombinant DNA technology methods. Vectors can be plasmids, phages, cosmids, phagemids, viruses, or any other types known in the art, used for replication and expression in prokaryotic or eukaryotic cells.

It will be appreciated by one of skill in the art that a wide variety of components known in the art may be included in the vectors of the present invention, including a wide variety of transcription signals, such as promoters and other sequences that regulate the binding of RNA polymerase onto the promoter. The operation of promoters is well known in the art.

Any promoter known to be effective in the cells in which the vector will be expressed can be used to initiate expression of the FP. Suitable promoters may be inducible or constitutive. Examples of suitable promoters include the SV40 early promoter region, the promoter contained in the 3′ long terminal repeat of Rous sarcoma virus, the HSV-1 (herpes simplex virus-1) thymidine kinase promoter, the regulatory sequences of the metallothionein gene, etc., as well as the following animal transcriptional control regions, which exhibit tissue specificity and have been utilized in transgenic animals: elastase I gene control region which is active in pancreatic acinar cells; insulin gene control region which is active in pancreatic beta cells, immunoglobulin gene control region which is active in lymphoid cells, mouse mammary tumor virus control region which is active in testicular, breast, lymphoid and mast cells, albumin gene control region which is active in liver, alpha-fetoprotein gene control region which is active in liver, alpha 1-antitrypsin gene control region which is active in the liver, beta-globin gene control region which is active in erythroid cells, myelin basic protein gene control region which is active in oligodendrocyte cells in the brain, myosin light chain-2 gene control region which is active in skeletal muscle, and gonadotropin releasing hormone gene control region which is active in the hypothalamus.

In one aspect of the invention, a mammal is genetically modified to produce the FP in its milk. Techniques for performing such genetic modifications are described in U.S. Pat. No. 6,013,857, issued Jan. 11, 2000, for “Transgenic Bovines and Milk from Transgenic Bovines.” The genome of the transgenic animal is modified to comprise a transgene comprising a DNA sequence encoding an FP operably linked to a mammary gland promoter. Expression of the DNA sequence results in the production of FP in the milk. The FP peptides may then be isolated by phase transition from milk obtained from the transgenic mammal. The transgenic mammal is preferably a bovine.

In another aspect of the invention, the inverse phase transition method is used for synthesizing compounds, such as peptides and oligonucleotides, by reacting an ELP-monomer with substituent 1, followed by conducting an ITC cycle to remove unreacted components, and repeating this cycle with substituents 2, 3, 4, . . . until the desired compound is synthesized. This method is useful for making large amounts of peptides that are traditionally difficult to cost-effectively synthesize on a large scale.

Method for Isolating and/or Partially Purifying Recombinant Proteins and Other Applications

The invention provides a method for isolating and/or partially purifying recombinantly produced proteins. The method generally comprises preparing a nucleotide sequence encoding the fusion protein, introducing the nucleotide sequence into cells of a cell culture, expressing the fusion protein in the cells of the cell culture, lysing the cells of the cell culture and isolating the FP from solution by inverse phase transition. Where the FP is secreted from live cells, it is not necessary to lyse the cells.

The FPs of the invention can be separated from other contaminating proteins to high purity using the inverse transition cycling (ITC) procedure of the present invention. Methods of isolation can employ the temperature-dependent solubility of the FP. The inventor has surprisingly discovered that soluble FP can be selectively aggregated by raising the solution temperature above the Tₜ with no effect on other soluble proteins present in the cell lysate. Successive inverse phase transition cycles may be used to obtain a correspondingly higher degree of purity.

Other purification techniques may also be employed in conjunction with the inverse phase transition. For example, recombinant cells may be designed to secrete the FP; the cells may be cultured in a cross-flow filter system that permits the secreted FP proteins to diffuse across a membrane. The FPs may then be purified from other contaminants by inverse phase transition.

Inverse phase transition can also be induced by depressing the Tₜ by manipulating other solution conditions. For example, the Tₜ can be adjusted so that soluble fusion protein can be isothermally aggregated at room temperature, for example, by the addition of salt. Because this process is reversible, altering the solution conditions back to the original conditions results in the recovery of soluble, pure, and functionally-active fusion protein.

The inverse transition of the ELP also provides a simple method for purifying the ELP tag from the target protein after cleavage at a protease recognition site encoded in the primary amino acid sequence between the target protein and the ELP carrier. After cleavage, the target protein is easily separated from free ELP by another round of inverse transition cycling.

In addition to temperature and ionic strength, other environmental variables useful for modulating the inverse transition of FPs include pH, the addition of inorganic and organic solutes and solvents, side-chain ionization or chemical modification, and pressure.

Although purification of recombinant proteins is the most obvious and immediate application of the FPs of the invention, the invention provides other applications in biotechnology and medicine.

In one embodiment, the protein component of the FP is an enzyme. Such enzyme-FPs (EFPs) may be used as substitutes for immobilized enzymes in industrial biocatalysis. The EFPs may be added to a solution to facilitate biocatalysis and then reisolated from the solution. The utilization of free EFPs rather than immobilized enzymes permits substantial increases in kinetics of the biocatalysis to be achieved. Furthermore, the EFPs facilitate both separation of the enzyme from product and recycling of the enzyme for subsequent rounds of biocatalysis.

Consider the following method for purifying a therapeutic protein in bulk comprising the forming of a polynucleotide sequence encoding a fusion protein including the therapeutic protein and a protein exhibiting a phase transition (ELP tag). The method includes the steps of (i) expressing the fusion protein in a transformed host cell; (ii) secreting the fusion protein from the host cells, or alternatively disrupting the host cells to release the fusion protein; (iii) aggregating the fusion protein by a method that comprises a phase transition, e.g., by raising temperature (ITC); (iv) concentrating the aggregated fusion protein by centrifugation; (v) discarding the supernatant and resolubilizing the pelleted fusion protein; (vi) adding an enzyme to cleave the therapeutic protein from its ELP-tag; (vii) aggregating the free ELP-tag by a method that comprises a phase transition, e.g., by raising temperature; (viii) concentrating the aggregated free ELP-tag by centrifugation; (ix) recovering the supernatant containing the purified therapeutic protein.

In another embodiment, the protein component of the FP is a ligand-binding protein, such as an antibody, that has binding affinity to a biomolecule of interest, such as small organic or inorganic molecules, proteins, peptides, single-stranded or double-stranded oligonucleotides, polynucleotides, lipids, and carbonhydrates. Such FPs containing the ligand-binding protein can be employed for capture and subsequent isolation of an analyte from a solution, such as a biological fluid, and are useful in immunoassays. The ligand-binding protein can be further labeled (e.g., radiolabelled, labeled with fluorescent or luminescent tags) to facilitate assays, such as immunoassays.

Another application of FPs of the invention is for targeted delivery of therapeutics and imaging agents, where in concert with targeted hyperthermia, FP conjugated to radionuclides or protein therapeutics enables precise targeting for imaging and therapy.

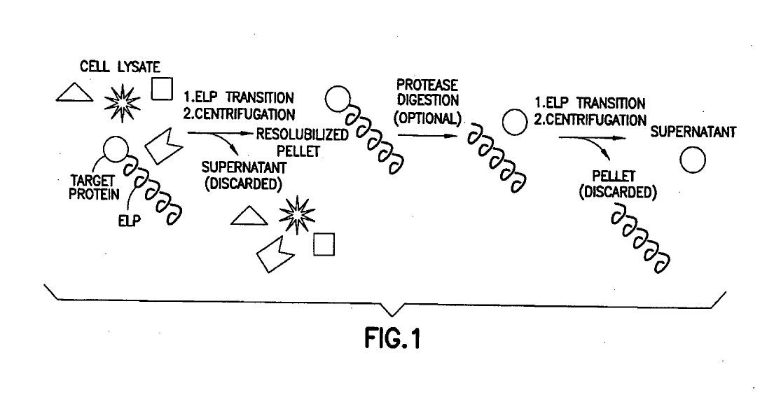

FIG. 1 schematically shows an inverse transition cycling (ITC) purification scheme. A target protein, which is genetically fused to an ELP, is separated from other contaminating proteins in the cell lysate after inducing the ELP inverse temperature phase transition. The solution is first cycled The solution is first cycled to above the Tₜ to selectively aggregate the target fusion protein so that it can be separated by centrifugation, and then cooled to below the Tₜ to resolubilize the purified fusion protein. The target protein can be liberated from the fused ELP tag by cleavage at a specific protease recognition site engineered between the ELP tag and the target protein. The cleaved ELP can be removed by a final round of ITC. After centrifugation, the purified target protein is obtained in the supernatant, while the aggregated ELP is discarded in the pellet.

ELP Optimization

The ELP tag size may be optimized to provide a desired inverse transition temperature (Tₜ). The ability to optimize Tₜ to a desired temperature enables the efficient recovery of expressed protein from recombinant organisms that are grown in culture. Consider an ELP tag sequence that allows the expressed fusion protein to remain soluble under culture conditions yet effect its aggregation in response to a small increase in temperature. Both ELP composition and chain length have been shown to strongly affect the Tₜ (Urry, D. W. et al. Temperature of polypeptide inverse temperature transition depends on mean residue hydrophobicity. J. Am. Chem. Soc. 113, 4346-4348 (1991); and Urry, D. W. et al. Phase-structure transitions of the elastin polypentapeptide water system within the framework of composition-temperature studies. Biopolymers 24, 2345-2356 (1985)).

As known to those of skill in the art, the preferred Tₜ will vary among organisms with respect to their temperature requirement for growth. Wherein, the preferred Tₜs permit solubility of FP in the recombinant organism during culture and aggregation of FP by a small increase in temperature following cell lysis. Preferably the temperature increase to effect aggregation is 1 to 5° C. Given a culture temperature of 37° C., the preferred Tₜ will be 40° C. To effect such a Tₜ an ELP residue composition was selected based on the previous studies of Urry et al. (Urry, D. W. et al. Temperature of Polypeptide Inverse Temperature Transition Depends on Mean Residue Hydrophobicity. J. Am. Chem. Soc. 113, 4346-4348 (1991)) with the preferred ELP pentapeptide Val-Pro-Gly-X-Gly, with guest residues Val, Ala and Gly in the ratio of 5:2:3.

Varying ELP chain length and ionic strength can also vary inverse transition temperatures. Moreover, ELP chain lengths are also important with respect to protein yields. Reducing the size of the ELP tag may be employed to substantially increase the yield of the target protein. However, truncation of the ELP tag results in more complex transition behavior than observed with larger tags. Since only large aggregates can be effectively retrieved by centrifugation, efficient purification of fusion proteins with short ELP tags requires selection of solution conditions that favor the formation of the larger aggregates. Despite this additional complexity, the size of the ELP tag can be optimized to enhance the yield of a target protein without compromising purification.

Genetically-encodable, environmentally-responsive ELP peptides may be expressed and screened for optimal activity as a function of solution environment. In such methodology, polynucleotides are employed that comprise a nucleotide sequence encoding a fusion protein that comprises the protein of interest and an ELP tag. The method comprises the steps of (i) forming a multiplicity of polynucleotides, each comprising a nucleotide sequence encoding a fusion protein exhibiting a phase transition, wherein each of such multiplicity of polynucleotides includes a different-sized ELP expression tag, (ii) expressing corresponding fusion proteins from such multiplicity of polynucleotides, (iii) determining a yield of the desired protein for each of the corresponding fusion proteins, (iv) determining size of particulates for each of the corresponding fusion proteins in solution as temperature is raised above Tₜ, and (v) selecting an optimized size ELP expression tag according to predetermined selection criteria, e.g., for maximum recoverable protein of interest from among said multiplicity of polynucleotides, or for achieving a desired balance between yield and ease of isolation ability for each of the proteins of interest produced from the respective polynucleotides.

The residue composition of the synthetic gene is based upon predetermined selection criteria (e.g., culture temperature) for the base polypeptide ELP. Standard molecular biology protocols are used for gene synthesis and oligomerization.

In a specific illustrative embodiment of the invention, a 10 polypentapeptide ELP (an ELP 10-mer) is constructed. The ELP 10-mer may be oligomerized or polymerized up to 18 times to create a library of ELPs with precisely specified molecular masses (10-, 20-, 30-, 60-, 90-, 120-, 150-, and 180-mers). The ELP polymers or oligomers may then be fused to the C- or N-terminus of the protein of interest. A second protein of interest may be fused to the ELP component of the fusion protein construct, providing a ternary fusion. Optionally, one or more spacers may be used to separate the ELP tag from the protein(s) of interest. Preferably, when the spacers are present, each spacer comprises a proteolytic cleavage site, which permits the ELP tag to be enzymatically cleaved to enable isolation and/or partial purification of the protein(s) of interest.

Microplate Format and High Throughput Purification Using ITC

The ITC purification technique of the invention can be scaled down to a microplate format (96-well). Growth or expression of a FP, and its subsequent purification using a microplate format can for example be carried out with purification efficiencies on the order of 8-20% of the expressed protein from the cell lysate, with net yields of 3-5 μg of target protein per well at a purity of 90% as determined by SDS-PAGE. Microplate protein growth and purification is readily carried out, e.g., by the steps of: (i) inoculating growth media with a transformed cell line; (ii) inducing the inoculated cell line to express the FP; (iii) harvesting the cells; (iv) lysing the cells; (v) centrifuging and retaining the supernatant; (vi) inducing an inverse transition cycle (ITC) by adding salt or increasing temperature; (v) centrifuging and discarding the supernatant; (vi) resuspending the pellet in a low salt buffer; and (vii) centrifuging and retaining the supernatant.

Further, the scaled down microplate format can be multiplexed for concurrent, parallel laboratory scale purification from numerous cell cultures, to achieve simultaneous purification of proteins from multiple cultures. Such high-throughput purification application of the invention can be utilized, for example, to expedite both structure-function studies of proteins and the screening of proteins in pharmaceutical studies.

### EXAMPLES

The principal features of the invention are more fully shown with illustrative reference to experiments involving the expression of fusion proteins containing various different recombinant proteins, such as thioredoxin, tendamistat, insulin, T20 protein, interferon, tobacco etch virus protease, small heterodimer partern orphan receptor, androgen receptor ligand binding protein, glucocorticoid receptor ligand binding protein, estrogen receptor ligand binding protein, G proteins, and 1-deoxy-D xylulose 5-phosphate reductoisomerase, that are fused to various different ELP sequences.

The results demonstrate a gentle, one-step separation of these fusion proteins from other soluble proteins in the cell lysate, by exploiting the inverse transition of the fusion proteins imparted by the ELP tags.

### Example 1

### Fusion Proteins Containing Thioredoxin and/or Tendamistat

Thioredoxin and tendamistat exemplify two limiting scenarios of protein expression: (1) the target protein over-expresses at high levels and is highly soluble (thioredoxin), and (2) the target protein is expressed largely as insoluble inclusion bodies (tendamistat). It is preferable that proteins representative of this second class exhibit some level of expression as soluble protein to be purified by inverse transition cycling.

The thioredoxin-ELP fusion protein exhibited only a small increase in Tₜ (1-2° C.) compared to free ELP, while the tendamistat fusion displayed a more dramatic 15° C. reduction in Tₜ. This shift was identical for both the ternary (thioredoxin-ELP-tendamistat) and binary (ELP-tendamistat) constructs, indicating that the Tₜ shift was associated specifically with tendamistat. These observations are consistent with the conclusion that the decreased Tₜ was due to interactions between the ELP chain and solvent-exposed hydrophobic regions in tendamistat, whereas, for the highly soluble thioredoxin, these hydrophobic interactions were negligible. Moreover, with highly soluble proteins only a small perturbation of Tₜ relative to the free ELP is likely to be introduced upon fusion with an ELP tag.

In order to demonstrate fundamental concepts of the invention, a gene encoding an ELP sequence was synthesized and ligated into two fusion protein constructs (shown schematically in FIG. 1b).

In the first construct, an ELP sequence was fused to the C-terminus of E. coli thioredoxin, a 109 residue protein that is commonly used as a carrier to increase the solubility of target recombinant proteins. In the second, more complex construct, tendamistat, a 77 residue protein inhibitor of α-amylase, was fused to the C-terminus of a thioredoxin-ELP fusion, forming a ternary fusion.

The objective in this example was to design a β-turn sequence with a predicted Tₜ above 37° C. so that an FP would remain soluble under conditions used for E. coli culture, but which could be aggregated by a small increase in temperature. Previous studies by Urry and colleagues have shown that two ELP-specific variables, guest residue(s) composition (i.e., identity and mole fraction of X in the VPGXG monomer) and chain length of the ELP profoundly affect the transition temperature, and thereby provide design criteria to specify the Tₜ for a specific application.

Based on these studies, a gene was synthesized encoding an ELP sequence (SEQ ID NO: 13) with guest residues valine, alanine, and glycine in the ratio 5:2:3, with a predicted Tₜ of ˜40° C. in water. The synthetic gene, which encoded 10 VPGXG pentapeptide repeats (the “10-mer”), was oligomerized up to 18 times to create a library of genes encoding ELPs with precisely-specified molecular weights (MWs) ranging from 3.9 to 70.5 kDa. To the inventor's knowledge, these are the first examples of genetically-engineered ELPs with precisely-defined chain length and amino acid sequence, which are designed to exhibit an inverse transition at a specified temperature. Thioredoxin was expressed as a N-terminal fusion with the 10-, 20-, 30-, 60-, 90-, 120-, 150-, and 180-mer ELP sequences, and tendamistat was expressed as a C-terminal fusion to thioredoxin/90-mer ELP (FIG. 1b).

The FPs were expressed in E. coli and purified from cell lysate either by immobilized metal affinity chromatography (IMAC) using a (histidine)₆ tag present in the fusion protein or by inverse transition cycling (described below). The purified FP was cleaved with thrombin to liberate the target protein from the ELP. The ELP was then separated from the target protein by another round of inverse transition cycling, resulting in pure target protein. For each construct, the purified FP, target protein, and ELP were characterized by sodium dodecyl sulfate polyacrylamide gel electrophoresis (SDS-PAGE), which confirmed protein purity, verified completeness of thrombin cleavage, and showed that the migration of each protein was consistent with its predicted size (results not shown).

The inverse transition of the fusion protein so formed can be spectrophotometrically-characterized by monitoring solution turbidity as a function of temperature, due to aggregation of the ELP-containing fusion protein as it undergoes the transition. As the temperature is raised up to a critical temperature, the solution remains clear. Further increase in temperature results in a sharp increase in turbidity over a ˜2° C. range to a maximum value (OD₃₅₀˜2.0). The Tₜ, defined as the temperature at the midpoint of the spectrophotometrically-observed transition, is a convenient parameter to describe this process.

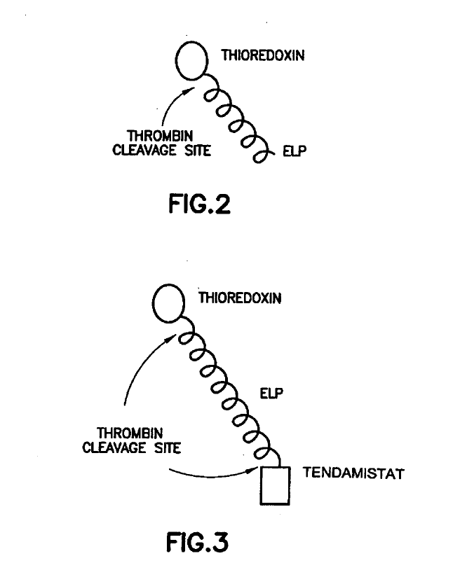

The inverse transition of free ELP, thioredoxin-ELP fusion, ELP-tendamistat fusion, and ternary thioredoxin-ELP-tendamistat fusion in PBS are shown in FIG. 2a. The Tₜ, was 51° C. for free ELP and 54° C. for the thioredoxin fusion, showing that the Tₜ is only slightly affected by fusion to thioredoxin. Thioredoxin-ELP produced by cleavage from the ternary tendamistat fusion had a higher Tₜ compared to thioredoxin-ELP produced directly (60° C. vs. 54° C.), presumably due to differences in the leader and trailer amino acid sequences immediately adjacent to the ELP sequence (see FIG. 5). The transition profiles of ELP-tendamistat and the thioredoxin-ELP-tendamistat were nearly identical, with a Tₜ of 34° C. Aggregation of the FPs was reversible, and the aggregates were resolubilized completely upon lowering the temperature below the Tₜ. However, resolubilization kinetics were slower for ELP-tendamistat and thioredoxin-ELP-tendamistat fusions, typically requiring 5 to 10 minutes versus only a few seconds for free ELP and thioredoxin-ELP. Thioredoxin and tendamistat controls exhibited no change in absorbance with increasing temperature, indicating that the thermally-induced aggregation observed for the fusion proteins was due to the inverse transition of the ELP carrier. Typically, the inverse transition of the fusion proteins was also slightly broader than that of free ELP, and small upper and lower shoulders were observed in their turbidity profiles.

Motivated by the studies of Urry and colleagues, who observed a decrease in Tₜ with increasing chain length, the effect of ELP MW on the inverse transition of FPs was also investigated. The Tₜ of a set of thioredoxin-FPs were determined as a function of the MW of the ELP carrier, which ranged from 12.6 to 71.0 kDa (FIG. 2b). The Tₜ's of the higher MW fusion proteins approached the design target temperature of 40° C. (42° C. for the 71 kDa ELP), while the Tt's for the lower MW fusions were significantly greater (e.g., 77° C. for the 12.6 kDa ELP).

In addition to ELP-specific variables that affect the Tₜ (i.e., guest residue composition and MW), the Tₜ can be further modulated for a given ELP by several extrinsic factors, such as the choice of solvent, ELP concentration, and ionic strength. Controlling the ionic strength, in particular, allows the Tₜ to be tuned over a 50° C. range (FIG. 2c), and thereby provides a convenient method to optimize the Tₜ of a given ELP for a specific application. Manipulating the solution temperature and ionic strength also provides experimental flexibility in inducing the inverse transition for a specific ELP by several methods: (1) by increasing the solution temperature above the Tₜ at a given ionic strength, (2) by increasing the ionic strength isothermally to reduce the Tₜ below solution temperature, or (3) by simultaneously changing the solution temperature and ionic strength.

The specific activity of the thioredoxin/60-mer FP, determined by an insulin reduction assay, was identical to that of commercially-available E. coli thioredoxin (results not shown), indicating that below the Tₜ, the ELP tag had no effect on thioredoxin activity. For the ternary thioredoxin-ELP-tendamistat fusion, an α-amylase inhibition assay showed that the thioredoxin/90-mer ELP carrier reduced the α-amylase inhibition activity of tendamistat by 2-fold (results not shown). However, after thrombin cleavage and purification of tendamistat from the thioredoxin-ELP carrier, the activity of purified tendamistat was indistinguishable from recombinant tendamistat, which was independently purified by IMAC.

The application of inverse transition cycling for protein purification requires that the phase transition of the ELP does not denature the target protein. The aggregation, resolubilization, and functional activity of the thioredoxin/60-mer ELP fusion upon thermally cycling in 1.5 M NaCl were therefore monitored (FIG. 3). 1.5 M NaCl was added to the buffer simply to lower the Tₜ (from 62° C. in water to 27° C.) so that the FP would undergo its inverse transition in each thermal cycle between the experimentally-convenient temperatures of 24 and 35° C. Before commencing thermal cycling, the solution temperature of 24° C. was below the Tₜ of the thioredoxin-FP, and the protein solution exhibited no detectable turbidity. The thioredoxin activity of the fusion protein was initially assayed at this temperature to establish a baseline. Upon increasing the temperature to 35° C., the fusion protein aggregated, resulting in increased turbidity (OD₃₅₀ 2.0). After lowering the temperature to 24° C., the solution cleared completely, indicating that the fusion protein had resolubilized. An aliquot was removed and assayed for thioredoxin activity, which was found to be identical to the initial value. This thermal cycling process was repeated twice. No change in activity was observed at 24° C. after each thermal cycle, which confirmed that the small temperature change and the resulting aggregation/resolubilization had no effect on protein stability and function. In addition, resolubilization and recovery of the aggregated fusion protein was quantitative and complete after lowering the temperature to 24° C.

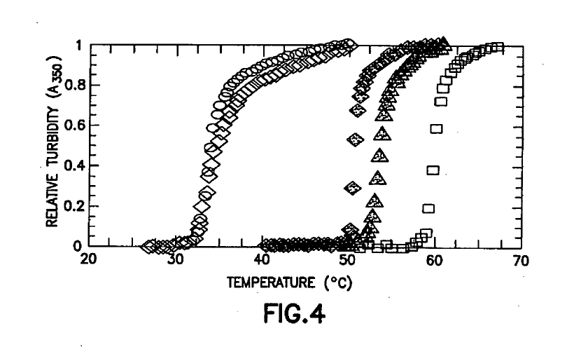

Six thioredoxin-FPs, where each fusion protein contained a C-terminal 30-, 60-, 90-, 120-, 150-, or 180-mer ELP tag, and the thioredoxin/90-mer ELP/tendamistat fusion protein were purified from cell lysate by inverse transition cycling, achieved by repeated centrifugation at conditions (i.e., NaCl concentration and temperature) alternating above and below the transition temperature. Typical SDS-PAGE results are shown in FIG. 4a for two rounds of inverse transition purification of thioredoxin/90-mer ELP (lanes 1-5) and for one round of purification of thioredoxin/90-mer ELP/tendamistat (lanes 7-9).

Before purification, the induced E. coli were harvested from culture media by centrifugation, resolubilized in a low salt buffer (typically PBS), and lysed by ultrasonic disruption. After high-speed centrifugation to remove insoluble matter, polyethylenimine was added to the lysate to precipitate DNA, yielding soluble lysate (lanes 1 and 7, FIG. 4a). Inverse transition cycling was then initiated by adding NaCl and/or increasing the solution temperature to induce the inverse transition of the FP, causing the solution to become turbid as a result of aggregation of the FP. The aggregated fusion protein was separated from solution by centrifugation at a temperature greater than the Tₜ, and a translucent pellet formed at the bottom of the centrifuge tube. The supernatant, containing contaminating E. coli proteins, was decanted and discarded (lanes 2 and 8). The pellet was redissolved in a low ionic strength buffer at a temperature below the Tₜ of the ELP, and centrifuged at low temperature to remove any remaining insoluble matter (lanes 3 and 9). Although additional rounds of inverse transition cycling were undertaken (lanes 4 and 5), the level of contaminating proteins was below the detection limit of SDS-PAGE after a single round of inverse transition cycling.

FIG. 4b shows the thioredoxin specific activity at each stage of purification of the thioredoxin/ELP fusion, as well as the total protein as estimated by BCA assay. Approximately 20% of the total protein in the soluble lysate (1) was precipitated in the first round of inverse transition purification (3), and the remaining soluble protein was decanted and discarded (2). The low thioredoxin activity measured in the supernatant, a portion of which is contributed by native E. coli thioredoxin, confirmed that this fraction primarily contained contaminating host proteins. The thioredoxin specific activity of the resolubilized protein approached that of commercially-available thioredoxin (data not shown), which confirmed that one round of inverse transition cycling resulted in complete purification. A second round of purification resulted in no detectable increase in thioredoxin specific activity (data not shown). The total thioredoxin activity after several rounds of inverse transition purification was experimentally-indistinguishable from that of the cell lysate (1, 3, and 5), indicating negligible loss of target protein in the discarded supernatant. These results quantitatively confirmed the high purity and efficient recovery of the thioredoxin-FP, and further demonstrated that functional activity of thioredoxin is fully retained after undergoing several rounds of inverse transition cycling.

Protein yields for the thioredoxin fusion constructs were typically greater than 50 milligrams of purified fusion protein per liter culture. The inventor found that the total gravimetric yield of fusion protein decreased with increasing ELP length, with the 30-mer (MW=12.6 kDa) averaging ˜70 mg/L and the 180-mer (MW=71.0 kDa) averaging ˜50 mg/L. Expression levels of soluble tendamistat were slightly larger for the ternary thioredoxin-ELP-tendamistat fusion (45 mg/L ternary fusion, or 7 mg/L tendamistat) compared to its fusion with thioredoxin only (10 mg/L thioredoxin-tendamistat fusion, 4 mg/L tendamistat).

As described hereinabove, two recombinant proteins, thioredoxin and tendamistat, fused to an environmentally-responsive ELP sequence, were expressed and a gentle, one-step separation of these fusion proteins from other soluble E. coli proteins was achieved by exploiting the inverse transition of the ELP sequence. Thioredoxin and tendamistat were selected as target proteins because they exemplify two limiting scenarios of soluble protein expression: (1) the target protein over-expresses at high levels and is highly soluble (thioredoxin), and (2) the protein is expressed largely as insoluble inclusion bodies (tendamistat). However, proteins representative of this latter class must exhibit some level of expression as soluble protein to be purified by inverse transition cycling.

Thioredoxin is expressed as soluble protein at high levels in E. coli, and is a therefore a good first test of whether the reversible, soluble-insoluble inverse transition of the ELP tag would be retained in a fusion protein. In contrast, tendamistat was selected as the other test protein because it is largely expressed as insoluble protein in inclusion bodies. Although fusion with thioredoxin is known to promote the soluble expression of target proteins, only 5-10% of over-expressed thioredoxin-tendamistat fusion protein was recovered as soluble and functionally-active protein. There was initial concern that incorporation of a hydrophobic ELP sequence in a fusion protein that exhibits a pronounced tendency to form inclusion bodies might (1) exacerbate its irreversible aggregation in vivo during culture, and (2) cause irreversible aggregation in vitro during purification by inverse transition cycling. Contrariwise, however, neither problem was encountered with the ELP-tendamistat fusion protein.

The ELP polypeptide tag used for thermally-induced, phase separation of the target recombinant protein was derived from polypeptide repeats found in mammalian elastin. Because the phase transition of ELPs is the fundamental basis of protein purification by inverse transition cycling, specifying the transition temperature is the primary objective in the design of an ELP tag.

Previous studies by Urry and colleagues have shown that the fourth residue (X) in the polypentapeptide sequence, VPGXG, can be altered without eliminating the formation of the β-turn, a structure that is advantageous to the inverse transition. These studies also showed that the Tₜ is a function of the hydrophobicity of the guest residue. Therefore, by varying the identity of the guest residue(s) and their mole fraction(s), ELP copolymers can be synthesized that exhibit an inverse transition over a 0-100° C. range. Based on these results, an amino acid sequence was selected to result in a predicted Tₜ of ˜40° C. in water, so that the ELP carrier would remain soluble in E. coli during culture but could be aggregated by a small increase in temperature after cell lysis.

In addition to the amino acid sequence, it is known that Tₜ also varies with ELP chain length. The design therefore incorporated precise control of molecular weight by a gene oligomerization strategy so that a library of ELPs with systematically varied molecular weight could be synthesized. The Tₜ's of the higher molecular weight ELPs approached the target temperature, with an experimentally-observed Tₜ of 42° C. for the thioredoxin/180-mer fusion (at 25 μM in PBS). However, the Tₜ increased dramatically with decreasing MW. In low ionic strength buffers, the Tₜ's of the lower molecular weight ELPs are too high for protein purification, and would consequently require a high concentration of NaCl to decrease the Tₜ to a useful temperature. ELP chain length is also important with respect to protein yields. In addition to the decreased total yield of expressed fusion protein observed with increasing ELP MW, the weight percent of target protein versus the ELP also decreases as the MW of the ELP carrier increases. Therefore, the design of the ELP tags of the present invention for purification preferably maximizes target protein expression by minimizing the ELP molecular weight, while retaining a target Tₜ near 40° C. through the incorporation of a larger fraction of hydrophobic guest residues in the ELP sequence.

The thioredoxin-ELP fusion as described hereinabove exhibited only a small increase in Tₜ (1-2° C.) compared to free ELP, while the tendamistat-ELP fusion displayed a more dramatic 15° C. reduction in Tₜ. This shift was identical for both the ternary (thioredoxin-ELP-tendamistat) and binary (ELP-tendamistat) constructs, indicating that the Tₜ shift is associated specifically with tendamistat. Based on these observations, it was hypothesize that the decreased Tₜ was due to interactions between the ELP chain and solvent-exposed hydrophobic regions in tendamistat, whereas, for the highly soluble thioredoxin, these hydrophobic interactions were negligible. Although this shift in Tₜ added complexity to the design of ELP carriers for inverse transition purification of proteins containing a significant fraction of exposed hydrophobic area, for highly soluble proteins only a small perturbation of Tₜ relative to the free ELP is likely to be introduced upon fusion with an ELP tag.

Standard molecular biology protocols were used for gene synthesis and oligomerization of the ELP tags. The synthetic gene for the 10-mer polypentapeptide VPGXG ELP was constructed from four 5′-phosphorylated, PAGE-purified synthetic oligonucleotides (Integrated DNA Technologies, Inc.), ranging in size from 86 to 97 bases. The oligonucleotides were annealed to form double-stranded DNA spanning the ELP gene with EcoRI and HindIII compatible ends (FIG. 5a). The annealed oligonucleotides were then ligated, using T4 DNA ligase, into EcoRI/HindIII linearized and dephosphorylated pUC-19 (NEB, Inc.). Chemically competent E. coli cells (XL1-Blue) were transformed with the ligation mixture, and incubated on ampicillin-containing agar plates. Colonies were initially screened by blue-white screening, and subsequently by colony PCR to verify the presence of an insert. The DNA sequence of a putative insert was verified by dye terminator DNA sequencing (ABI 370 DNA sequencer).

First, a 20-mer ELP gene was created by ligating a 10-mer ELP gene into a vector containing the same 10-mer ELP gene. The 20-mer gene was similarly combined with the original 10-mer gene to form a 30-mer gene. This combinatorial process was repeated to create a library of genes encoding ELPs ranging from 10-mer to 180-mer polypentapeptides. For a typical polymerization or oligomerization, the vector was linearized with PflMI and enzymatically dephosphorylated. The insert was doubly digested with PflMI and BglI, purified by agarose gel electrophoresis (Qiaex II Gel Extraction Kit, Qiagen Inc.), ligated into the linearized vector with T4 DNA ligase, and transformed into chemically competent E. coli cells. Trans formants were screened by colony PCR, and further confirmed by DNA sequencing.

For the thioredoxin fusion proteins, pET-32b expression vector (Novagen Inc.) was modified to include an SfiI restriction site and a transcriptional stop codon downstream of the thioredoxin gene (FIG. 5b). For the ternary tendamistat fusion, a previously constructed pET-32a based plasmid containing a gene for a thioredoxin-tendamistat fusion was modified to contain an SfiI restriction site in two alternate locations, upstream or downstream of the thrombin recognition site (FIG. 5c). ELP gene segments, produced by digestion with PflMI and BglI, were then ligated into the SfiI site of each modified expression vector. Cloning was confirmed by colony PCR and DNA sequencing.

The expression vectors were transformed into the expression strains BLR(DE3) (for thioredoxin fusions) or BL21-trxB(DE3) (for tendamistat fusion) (Novagen, Inc.). Shaker flasks with 2×YT media, supplemented with 100 μg/ml ampicillin, were inoculated with transformed cells, incubated at 37° C. with shaking (250 rpm), and induced at an OD₆₀₀ of 0.8 by the addition of isopropyl α-thiogalactopyranoside (IPTG) to a final concentration of 1 mM. The cultures were incubated an additional 3 hours, harvested by centrifugation at 4° C., resolubilized in low ionic strength buffer (˜1/30 culture volume), and lysed by ultrasonic disruption at 4° C. The lysate was centrifuged at ˜20,000×g at 4° C. for 15 minutes to remove insoluble matter. Nucleic acids were precipitated by the addition of polyethylenimine (0.5% final concentration), followed by centrifugation at ˜20,000×g at 4° C. for 15 minutes. Soluble and insoluble fractions of the cell lysate were then characterized by sodium-dodecyl sulfate polyacrylamide gel electrophoresis (SDS-PAGE).

The thioredoxin-ELP fusions, which contained a (His)₆ tag, were purified by immobilized metal ion affinity chromatography (IMAC) using a nickel-chelating nitrilotriacetic derivatized resin (Novagen Inc.) or alternatively by inverse transition cycling. The tendamistat-ELP fusion was purified exclusively by inverse transition cycling. For purification by inverse transition cycling, FPs were aggregated by increasing the temperature of the cell lysate to ˜45° C. and/or by adding NaCl to a concentration ˜2 M. The aggregated fusion protein was separated from solution by centrifugation at 35-45° C. at 10-15,000×g for 15 minutes. The supernatant was decanted and discarded, and the pellet containing the fusion protein was resolubilized in cold, low ionic strength buffer. The resolubilized pellet was then centrifuged at 4° C. to remove any remaining insoluble matter.

The optical absorbance at 350 nm of ELP fusion solutions were monitored in the 4-80° C. range on a Cary 300 UV-visible spectrophotometer equipped with a multi-cell thermoelectric temperature controller. The Tₜ was determined from the midpoint of the change in optical absorbance at 350 nm due to aggregation of FPs as a function of temperature at a heating or cooling rate of 1.5° C. min⁻¹. SDS-PAGE analysis used precast Mini-Protean 10-20% gradient gels (BioRad Inc.) with a discontinuous buffer system. The concentration of the fusion proteins was determined spectrophotometrically using calculated extinction coefficients. Total protein concentrations were determined by BCA assay (Pierce). Thioredoxin activity was determined by a calorimetric insulin reduction assay. Tendamistat activity was determined by a colorimetric α-amylase inhibition assay (Sigma).

The inventor has also synthesized ELP-GFP fusion proteins, where the ELP 90-mer and 180-mer were fused either N-terminal or C-terminal to green fluorescent protein (GFP) or its variant—blue fluorescent protein (BFP). All fusion polypeptides exhibited a reversible inverse transition as characterized by UV-vis spectrophotometric measurement of turbidity as a function of temperature, as well as temperature dependent fluorescence measurement. The inverse transition of the GFP-ELP and BFP-ELP fusions, was used to purify these fusion proteins to homogeneity by ITC, and was verified by SDS-PAGE and Coomassie staining.

Standard molecular biology protocols were further used for synthesis and polymerization/oligomerization of the ELP genes with reduced ELP molecular weight (Ausubel, et al.). Monomer genes for two ELP sequences were utilized in this example.

The first, ELP1 [V₅A₂G₃-10] encoding ten Val-Pro-Gly-Xaa-Gly repeats where Xaa was Val, Ala, and Gly in a 5:2:3 ratio (SEQ ID NO: 13), respectively, had been synthesized previously. The second monomer, ELP1 [V-5] (SEQ ID NO: 14), encoded five Val-Pro-Gly-Val-Gly pentapeptides (i.e., Xaa was exclusively Val). The coding sequence for the ELP1 [V-5] monomer gene was: 5′-GTGGGTGTTCCGGGCGTAGGTGTCCCAGGTGTGGGCGTACCGGGCGTTGGTGTTCCTG GTGTCGGCGTGCCGGGC-3′ (SEQ ID NO: 15). The monomer genes were assembled from chemically synthesized, 5′-phosphorylated oligonucleotides (Integrated DNA Technologies, Coralville, Iowa), and ligated into a pUC19-based cloning vector. A detailed description of the monomer gene synthesis is presented elsewhere.

The monomer genes for both ELP sequences, ELP1 [V₅A₂G₃-10] and ELP1 [V-5], were seamlessly oligomerized by tandem repetition to encode libraries of increasing ELP molecular weight. A detailed description of the gene oligomerization, using a methodology termed “recursive directional ligation”, is presented elsewhere. Briefly, an ELP gene segment (the monomer gene initially and larger multiples of the monomer in later rounds) is excised by restriction digest from its vector, purified, and ligated into a second cloning vector containing the same or a different ELP gene segment, thereby concatenating the two gene segments. This process can be repeated recursively, doubling the gene length with each round.

Different ELP constructs are distinguished here using the notation ELPk [XᵢYⱼ-n], where k designates the specific type of ELP repeat unit, the bracketed capital letters are single letter amino acid codes and their corresponding subscripts designate the relative ratio of each guest residue X in the repeat units, and n describes the total length of the ELP in number of the pentapeptide repeats. The two ELP constructs central to the present example are ELP1 [V₅A₂G₃-90] (35.9 kDa) (SEQ ID NO: 16) and ELP1 [V-20] (9.0 kDa) (SEQ ID NO: 17).

To produce the thioredoxin fusion proteins, genes encoding ELP1 [v₅A₂G₃-90] and ELP1 [V-20] were excised from their respective cloning vectors and separately ligated into a pET-32b expression vector (Novagen, Madison, Wis.), which had been previously modified to introduce a unique Sfi I site located 3′ to the thioredoxin gene, a (His)₆ tag, and a thrombin protease cleavage site. The modified pET32b vector encoding free thioredoxin with no ELP tag (“thioredoxin(His₆)”) and the two expression vectors encoding each fusion protein (“thioredoxin-ELP1 [V₅A₂G₃-90]” and “thioredoxin-ELP1 [V-20]”) were transformed into the BLR(DE3) E. coli strain (Novagen).

For quantitative comparison of the protein expression levels and purification yields, the three constructs were each expressed and purified in parallel. For each sample (four samples each of thioredoxin(His₆), thioredoxin-ELP1 [V-20], and thioredoxin-ELP1 [V₅A₂G₃-90]), a 2 ml starter culture (CircleGrow media, Qbiogene, Carlsbad, Calif., supplemented with 100 μg/ml ampicillin) was inoculated with a stab taken from a single colony on a freshly streaked agar plate, and incubated overnight at 37° C. with shaking at 300 rpm. To remove β-lactamase from the media, the cells were then pelleted from 500 μl of the confluent overnight culture by centrifugation (2000×g, 4° C., 15 min), resuspended in fresh media wash, and repelleted. After a second resuspension in fresh media, the cells were used to inoculate 50 ml expression cultures in 250 ml flasks (CircleGrow media with 100 μg/ml ampicillin).

The culture flasks were incubated at 37° C. with shaking at 300 rpm. Growth was monitored by the optical density at 600 nm, and protein expression was induced at OD₆₀₀=1.0 by the addition of isopropyl β-thiogalactopyranoside (IPTG) to a final concentration of 1 mM. After a further 3 hours of culture, the cells were harvested from 40 ml by centrifugation (2,000×g, 4° C., 15 min), resuspended in 2 ml of IMAC binding buffer (5 mM imidazole, 500 mM NaCl, 20 mM Trix-HCl, pH 7.9) for thioredoxin(His₆) or PBS (137 mM NaCl, 2.7 mM KCl, 4.2 mM Na₂HPO₄, 1.4 mM KH₂PO₄, pH 7.3) for thioredoxin-ELP1 [V-20] and thioredoxin-ELP1 [V₅A₂G₃-90], and stored frozen at −20° C. until purified. The culture density at harvest was measured by OD₆₀₀, after 1:10 dilution in fresh buffer. The amount of plasmid DNA at harvest was quantified by UV-visible spectrophotometry following plasmid isolation (plasmid miniprep spin kit, Qiagen, Valencia, Calif.).

As a control for ITC purification of the thioredoxin-ELP fusion proteins, free thioredoxin was purified using standard IMAC protocols. Briefly, the thawed cells were transferred to iced 15 ml centrifuge tubes and lysed by ultrasonic disruption (Fisher Scientific 550 Sonic Dismembrator using a microtip). After transferring to 1.5 ml micro centrifuge tubes, the E. coli lysate was centrifuged (16,000×g, 4° C., 30 min) to remove the insoluble cellular debris. 1 ml of the soluble cell lysate was loaded by gravity flow onto a column packed a 1 ml bed of nitrilotriacetic acid resin that had been charged with 5 ml of 50 mM NiSO₄.

After the column was washed with 15 ml of IMAC binding buffer, thioredoxin(His₆) was eluted in 6 ml of IMAC binding buffer supplemented with 250 mM imidazole. Imidazole was removed from the eluent by dialysis against a low salt buffer (25 mM NaCl, 20 mM Tris-HCl, pH 7.4) overnight using a 3,500 MWCO membrane. The IMAC purification was monitored by SDS-PAGE using precast 10-20% gradient gels (BioRad Inc., Hercules, Calif.) with a discontinuous buffer system.

The yield of the purified thioredoxin(His₆) was determined by spectrophotometry, using a molar extinction coefficient of thioredoxin modified to include the absorption of the single Trp residue present in the C-terminal tag (ε₂₈₀=19870 M⁻¹ cm⁻¹ for thioredoxin(His₆) and all thioredoxin-ELP fusion proteins, independent of ELP molecular weight.

In a typical purification by ITC, the thawed cells were transferred to iced 15 ml centrifuge tubes and lysed by ultrasonic disruption (Fisher Scientific 550 Sonic Dismembrator with a microtip). After transferring to 1.5 ml micro centrifuge tubes, the E. coli lysate was centrifuged at 4° C. for 30 min to remove the insoluble cellular debris. (All centrifugation steps during purification by ITC were performed at 16,000×g in Eppendorf 5415C microcentrifuges.)

Polyethylenimine was added (to 0.5% w/v) to the decanted supernatant of the cell lysate to precipitate nucleic acids, which were removed by an additional 20 min centrifugation at 4° C. The supernatant was retained, and the ELP phase transition was induced by increasing the NaCl concentration by 1.3 M. The aggregated fusion protein was separated from solution by centrifugation at 33° C. for 5 min, which resulted in the formation of translucent pellet at the bottom of the tube.

The supernatant was decanted and discarded, and the pellet containing the fusion protein was redissolved in an equal volume of PBS at 4° C. Any remaining insoluble matter was removed by a final centrifugation step at 4° C. for 15 min, and the supernatant containing the purified fusion protein was retained. The progression of fusion protein purification was monitored by SDS-PAGE, and the protein concentrations were determined by spectrophotometry, as described above for IMAC purification.

Thioredoxin was liberated from its ELP fusion partner using thrombin protease (Novagen), which cleaved the fusion protein at a recognition site located between thioredoxin and the ELP tag. The thrombin proteolysis reaction was allowed to proceed overnight at room temperature in PBS using ˜10 units of thrombin per μmol of fusion protein, which was typically at a concentration of ˜100 μM. Free ELP was then separated from the cleaved thioredoxin by another round of ITC, this time retaining the supernatant that contained the product thioredoxin.

The inverse transition can be monitored by assaying solution turbidity photometrically as a function of temperature, taking advantage of the fact that increase in temperature beyond a critical point results in a sharp increase in turbidity over an approximately 2° C. range to a maximum value (OD₃₅₀ approximately 2.0), because of aggregation of the ELP. The temperature at 50% maximal turbidity, T_{b}, is a convenient parameter for quantitatively monitoring the aggregation process.

The temperature-dependent aggregation behaviors of the thioredoxin-ELP fusion proteins were characterized by measuring the optical density at 350 nm as a function of temperature. Fusion proteins at concentrations typical of those found in the E. coli lysate during protein purification (160 μM for thioredoxin-ELP1 [V-20] and 40 μM for thioredoxin-ELP1 [V₅A₂G₃-90]) were heated or cooled at a constant rate of 1° C. min⁻¹ in a Cary Bio-300 UV-visible spectrophotometer (Varian Instruments, Walnut Creek, Calif.), which was equipped with a thermoelectric temperature-controlled multicell holder. The experiments were performed in PBS variously supplemented with additional NaCl. The ELP Tₜ was defined as the temperature at which the optical density reached 5% of the maximum optical density at 350 nm.

Dynamic light scattering (DLS) was used to monitor the particle size distribution of the thioredoxin-ELP fusion proteins as a function of temperature and NaCl concentration. Samples were prepared to reflect the protein and solvent compositions used in the turbidity measurements described above, and were centrifuged at 4° C. and 16,000×g for 10 minutes to remove air bubbles and insoluble debris. Prior to particle size measurement, samples were filtered through a 20 nm Whatman Anodisc filter at a temperature below the T.

Autocorrelation functions were collected using a DynaPro-LSR dynamic light scattering instrument (Protein Solutions, Charlottesville, Va.) equipped with a Peltier temperature control unit. Analysis was performed using Protein Solutions' Dynamics software version 5.26.37 using its regularization analysis for spherical particles. Light scattering data were collected at regular temperature intervals (either 1 or 2° C.) as solutions were heated from 20° to 60° C. Data were collected at each temperature by ramping the cell up to the temperature of interest, allowing the sample temperature equilibrate for at least 1 minute, and collecting 10 measurements, each with a 5 second collection time.

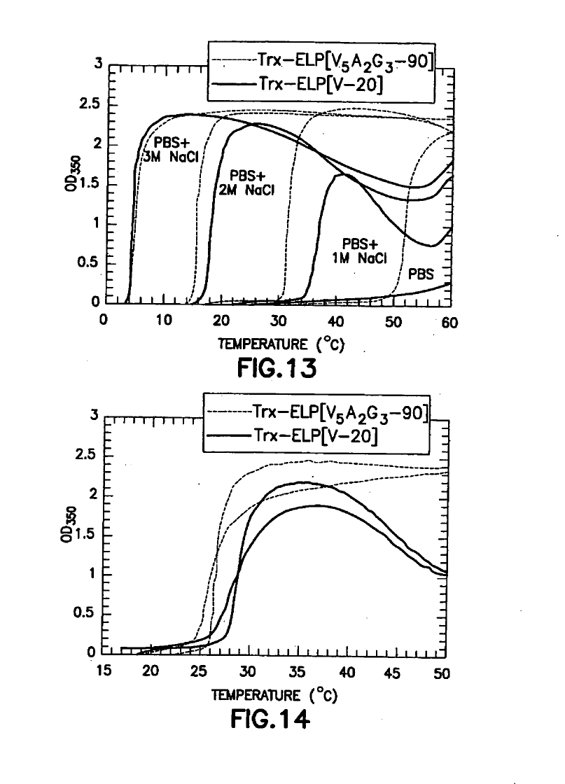

The inverse transition of each thioredoxin-ELP fusion protein in solution was characterized by monitoring the optical density at 350 nm as a function of temperature. Because different NaCl solutions are routinely used during ITC purification to depress the Tₜ or isothermally trigger the inverse transition, turbidity profiles were obtained for 40 μM thioredoxin-ELP1 [V₅A₂G₃-90] and 160 μM thioredoxin-ELP1 [V-20] in PBS and in PBS with an additional 1M, 2M, and 3M NaCl (FIG. 13).

FIG. 13 is a graph of optical density at 350 nm as a function of temperature for solutions of the thioredoxin-ELP fusion proteins. The turbidity profiles were obtained for thioredoxin-ELP1 [V-20] (solid lines) and thioredoxin-ELP1 [V₅A₂G₃-90] (dashed lines) in PBS, and in PBS supplemented with 1, 2, and 3 M NaCl, while heating at a rate of 1° C. min⁻¹. The concentration of thioredoxin-ELP1 [V₅A₂G₃-90] was 40 μM in each of the four PBS solutions, and that of thioredoxin-ELP1 [V-20] was 160 μM, which matched the typical concentration of each protein in the soluble cell lysate during ITC purification. All solutions showed a rapid rise in turbidity as they were heated through the Tₜ, but with continued heating beyond the Tₜ, the thioredoxin-ELP1 [V-20] solutions eventually became less turbid while the thioredoxin-ELP1 [V₅A₂G₃-90] solutions remained consistently turbid. All solutions of thioredoxin-ELP1 [V₅A₂G₃-90] cleared fully upon cooling the solution to below the Tₜ. However, solutions of ELP1 [V-20] cleared reversibly only if the solutions were not heated to above ˜55° C., suggesting thermal denaturation of the thioredoxin fusion protein occurred above this temperature. For clarity, only the heating profiles are shown.

The protein concentrations shown in FIG. 13 were chosen because they are typical of the concentrations obtained for each fusion protein in the soluble fraction of E. coli lysate, the stage at which the ELP inverse transition is first induced during ITC purification. Turbidity profiles obtained directly in the E. coli soluble cell lysate, supplemented with 1 and 2 M NaCl, were indistinguishable from the corresponding profiles in FIG. 13 (data not shown). (Turbidity profiles were not routinely obtained in E. coli lysate because of the potential for turbidity arising from thermal denaturation of E. coli proteins, which could not be differentiated from turbidity arising from aggregation of the ELP fusion protein.) Turbidity profiles were also obtained for each fusion protein in PBS with 1.3 M salt (FIG. 14), which matches the conditions used for the ITC purification described below.

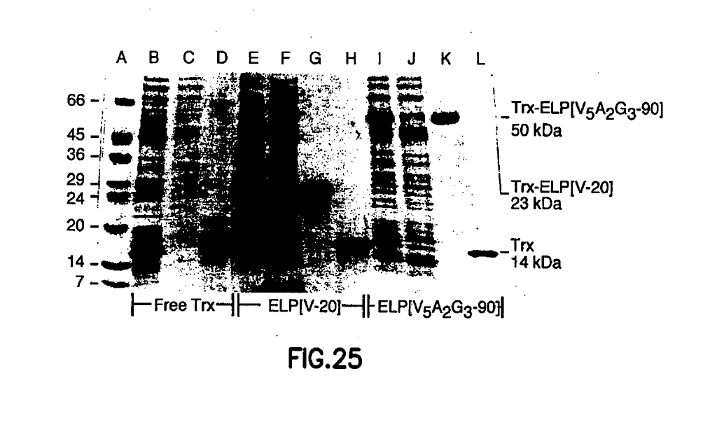

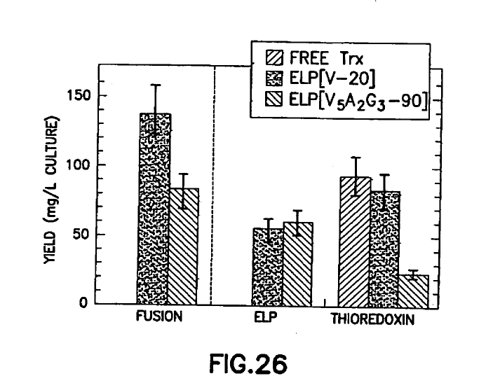

FIG. 14 is a graph showing the heating and cooling turbidity profiles for the solution conditions used in ITC purification, for solutions of thioredoxin-ELP1 [V-20] (solid lines) and thioredoxin-ELP1 [V₅A₂G₃-90] (dashed lines) at lysate protein concentrations in PBS with 1.3 M NaCl, corresponding to ITC conditions used for the quantitative comparison of expression and purification (FIGS. 25 and 26). These conditions were chosen so that the maximum turibidity of the thioredoxin-ELP1 [V-20] solution occurred at the centrifugation temperature of 33° C. The solutions were heated and cooled at 1° C. min⁻¹. The slight path differences between the heating and cooling curves were primarily due to slow settling of the aggregates over time at temperatures above Tₜ, and to the slower kinetics of disaggregation versus aggregation as the solutions are cooled to below Tₜ.

The thermally induced aggregation behavior of thioredoxin-ELP1 [V₅A₂G₃-90] was similar to that of free ELPs. All four salt concentrations, as the temperature of the thioredoxin-ELP1 [V₅A₂G₃-90] solutions was increased, remain clear until they reach the ELP Tₜ, at which point the turbidity sharply increased. This occurred at 51, 31, 15, and 4° C. in PBS with 0, 1, 2, and 3 M added NaCl, respectively. A free thioredoxin control solution exhibited no change in turbidity with increasing temperature over this temperature range, indicating that the thermally induced aggregation observed was due to the inverse transition of the ELP tag (results not shown). As these solutions were heated further beyond the Tₜ, the turbidity level remained essentially constant, and was only slightly reduced by settling of the aggregates over time. Upon cooling to below the Tₜ, the aggregates resolubilize and the optical density returned to zero, showing that the inverse transition of the ELP1 [V₅A₂G₃-90] fusion protein was completely reversible (for clarity, cooling traces are not shown in FIG. 13; however, an example of reversibility upon cooling is shown in FIG. 14). While increasing the NaCl concentration markedly decreases the Tₜ, salt has no measurable effect on the maximum optical density, on the general shape of the turbidity profiles, or on the reversibility of the aggregation.

In contrast, the phase transition behavior of thioredoxin-ELP1 [V-20] was considerably more complex than for the thioredoxin-ELP1 [V₅A₂G₃-90] fusion protein and free ELPs. Although the initial rapid rise in turbidity at the Tₜ (33, 17, and 4° C. in PBS supplemented with 1, 2, and 3 M NaCl, respectively) was similar to the other ELP constructs, the maximum turbidity observed with each of the thioredoxin-ELP1 [V-20] solutions increased with increasing salt concentration. Furthermore, increases in temperature beyond the Tₜ eventually resulted in a significant decrease in turbidity. This decrease was reversible; if the solution was cooled after heating to the point of decreased turbidity, the turbidity again increased (as illustrated in FIG. 3). Because the clearing phenomenon is a reversible function of temperature, it was concluded that a second, thermodynamically driven molecular rearrangement occurs with increasing temperature after the initial ELP aggregation event at T.

Another unique feature of the thioredoxin-ELP1 [V-20] turbidity profiles was a second increase in turbidity beginning at ˜55° C. (FIG. 13), which may have been due to aggregation arising from the irreversible thermal denaturation of thioredoxin. Samples heated to less than 55° C. reversibly cleared upon cooling to below the Tₜ (e.g., as in FIG. 14), whereas samples that are heated to above 55° C., for salt concentrations of 1 M and greater, remained turbid even upon cooling to below the Tₜ (not shown). This phenomenon appeared to be unique to the thioredoxin-ELP1 [V-20] fusion protein, as solutions of free thioredoxin and of its fusion proteins to larger ELPs were stable to much higher temperatures (results not shown). No inverse transition was observed for thioredoxin-ELP1 [V-20] in PBS below 60° C., however, with added salt the Tₜ was depressed so that it occured below the denaturation temperature in the PBS+1, 2, and 3 M NaCl solutions.

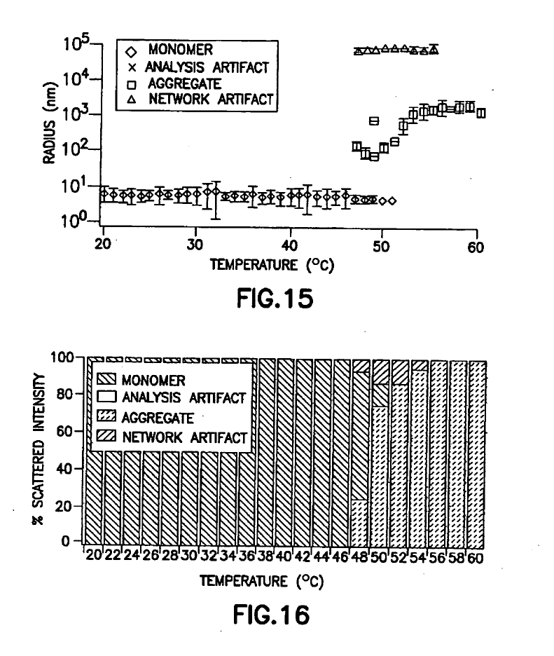

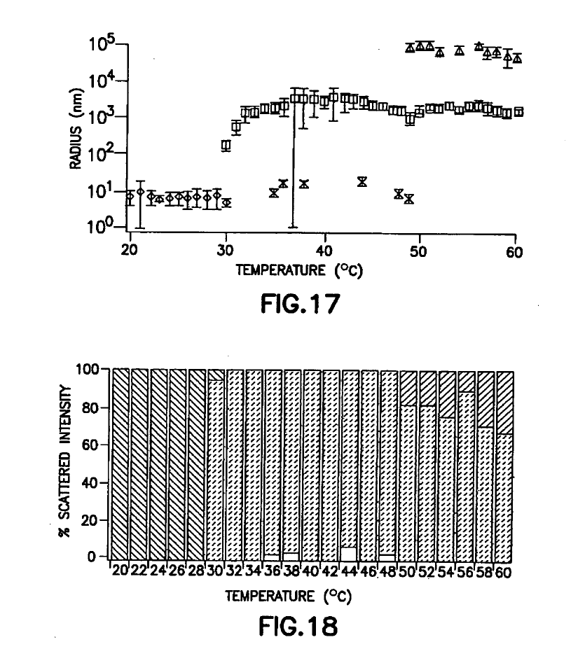

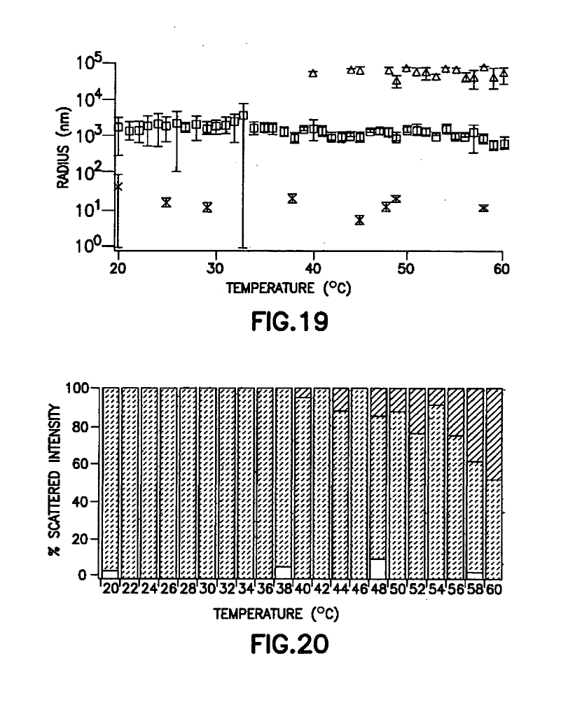

The sizes of the fusion protein particles were measured using DLS as a function of temperature. FIGS. 15-20 show the effect of temperature and salt on the particle size distribution (radius of hydration, Rₕ) of 40 μM thioredoxin-ELP1 [V₅A₂G₃-90] in PBS (FIGS. 15 and 16), PBS+1 M NaCl (FIGS. 17 and 18), and PBS+2 M NaCl (FIGS. 19 and 20). FIGS. 15, 17 and 19 show the effect of temperature on particle sizes of monomers (diamonds) and aggregates (squares). Analysis artifacts (stars) and network contributions (triangles), which may result from the coordinated slow movements of a network of smaller particles, are also shown (see text for discussion). FIGS. 16, 18 and 20 show the percentage of the scattered intensity attributed to each type of particle as a function of temperature. The appearance of the large aggregates closely coincided with the rise in turbidity observed in FIG. 13.

The sizes of thioredoxin-ELP1 [V₅A₂G₃-90] particles in PBS (FIG. 15), PBS with 1M added NaCl (FIG. 17), and PBS with 2M added NaCl (FIG. 19) indicate that the sharp increase in turbidity at the Tₜ resulted from the conversion of monomers with hydrodynamic radii (Rh) of 5.9±3.9 nm to aggregates with Rₕ of 180±62 nm. These aggregates grew with temperature until reaching a stable Rₕ of 2.2±3.8 μm approximately 6° C. above the onset of the transition. Although the Tₜ was depressed by the addition of NaCl, the sizes of both monomers and fully formed aggregates were not significantly affected by either the salt concentration or the temperature (outside the range immediately adjacent to the Tₜ), providing a rationale for the steady-state turbidity above the inverse Tt. The temperature at the onset of large aggregate formation closely matched the Tₜ determined by the turbidity measurements for corresponding solution conditions.

The corresponding quantitative breakdown of scattered intensity attributed to each type of particle is also shown for each of the salt concentrations investigated (FIGS. 16, 18 and 20). When two or more phases coexist over a given temperature range, these data show shifts in the relative particle populations. It should be noted that the intensity attributed to a particular population was not linearly correlated with the mass of that population, and that calculating the relative masses of multiple particles was complicated by changes in packing density that would likely accompany the inverse phase transition. Without a more detailed understanding of how temperature affects the packing density of ELPs and ELP fusion proteins, it was not possible to make a reasonable estimate for the mass attributed to each type of particle. Given these quantitative limitations, this data nonetheless shows that at the Tₜ the amount of scattered light attributed to the aggregate dramatically increased at the expense of the monomer.

FIGS. 15-20 also shows the occasional presence of both an unidentified small particle (with apparent Rₕ=17±31 nm, albeit highly variable) and an extremely large aggregate (with apparent Rₕ=74±55 μm) coexisting with the 2 μm aggregates. It is unlikely that the small particle is a true component of the aggregate suspension; rather, its presence reflects an artifact in the regularization algorithm resulting from noise in the autocorrelation function. Assignment as an analysis artifact is supported by the small particle's highly variable size and by its inconsistent presence at temperatures above the transition. Likewise, because its apparent size is much larger than can be discerned by the DLS instrument, it is also unlikely that the extremely large aggregate predicted from the data analysis represented a true species in suspension. Rather, the scattering attributed to this species may result from the coordinated slow movements of a network of smaller particles.

In contrast to thioredoxin-ELP1 [V₅A₂G₃-90], the smaller thioredoxin-ELP1 [V-20] fusion protein showed a more complicated temperature-dependent particle size distribution, which was consistent with its more complex turbidity profile.

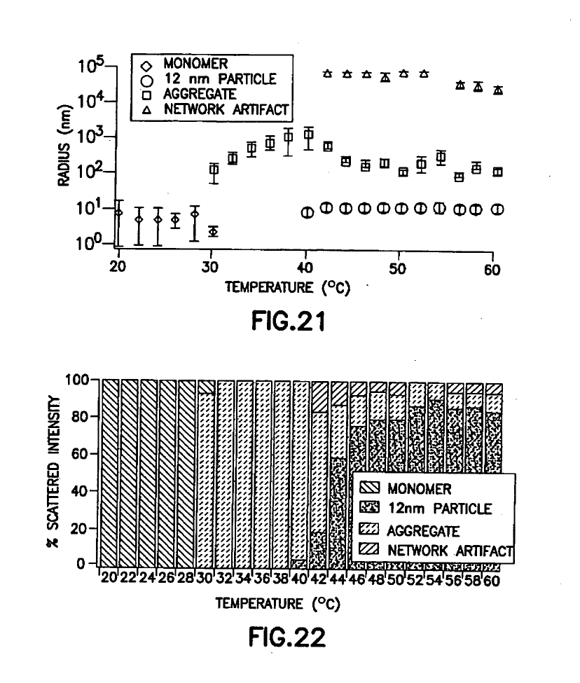

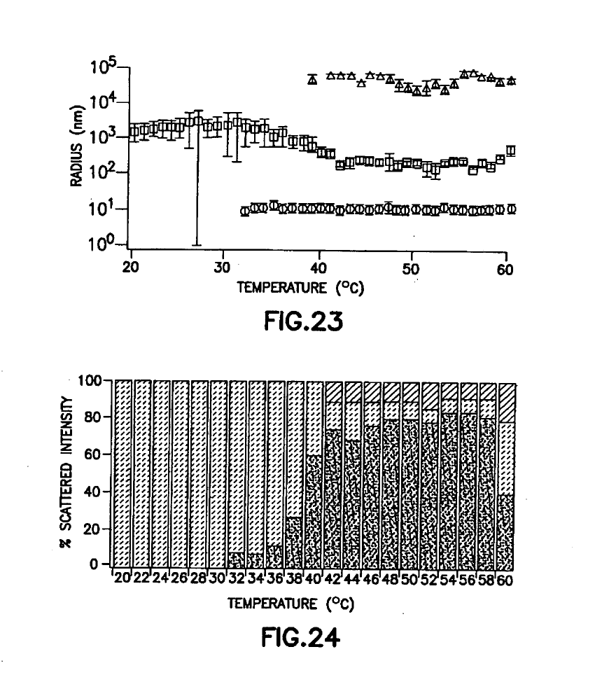

FIGS. 21-24 show the effect of temperature on the particle size distribution of ELP1 [V-20] in PBS+1 M NaCl (FIGS. 21 and 22) and PBS+2 M NaCl (FIGS. 23 and 24). FIGS. 21 and 23 show the effect of temperature on particle sizes of monomers (diamonds), 12 nm particles (circles), and larger aggregates (squares). Network contributions are also shown (triangles). FIGS. 22 and 24 show the percentage of the scattered intensity attributed to each type of particle as a function of temperature. The clearing in turbidity when the temperature is increased beyond Tₜ, as seen in FIG. 13, coincided with the shifting of mass from large aggregates to a new, smaller particle (Rₕ=12 nm).

Specifically, FIGS. 21-24 show the effects of salt and temperature on the distribution of the particle Rh and the corresponding contribution of each particle population to scattered intensity of 160 μM thioredoxin-ELP1 [V-20] in PBS with 1M and 2M added NaCl. For thioredoxin-ELP1 [V-20] with 1M added salt (FIG. 21) monomers with Rh of 5.9±5.1 nm were converted to aggregates with Rₕ of 140±79 nm at 30° C., corresponding in FIG. 13 to a small shoulder that precedes the rapid increase in turbidity at Tₜ. Above 30° C., aggregates grew with increasing temperature (up to Rₕ=1.5±0.98 μm at 40° C.), which was consistent with the rapid increase in turbidity observed starting at 33° C. in FIG. 13. Similar to the aggregation behavior of the large fusion protein, at temperatures greater than 40° C. thioredoxin-ELP1 [V-20] in PBS with 1 M added NaCl showed the presence of very large aggregates (apparent Rₕ=64±67 μm) that may reflect the coordinated slow movements of a network of smaller particles.

However, unlike the larger fusion protein, thioredoxin-ELP1 [V-20] also showed the consistent presence of a previously unobserved small particle at temperatures above 40° C. This particle had a Rₕ of 12±4.9 nm, which was roughly twice that of the monomer. Yet, relative to its mean Rₕ, its variability was only one half that of the monomer. The size, consistency, and continuous presence of this particle above 40° C. indicated that it was neither an analysis artifact resulting from noise in the autocorrelation function nor was it resolvated monomer. The 12 nm particle appeared to form at the expense of mass in the aggregates initially present above Tₜ, as evidenced by the reduction in size and scattering intensity of the larger aggregates (Rh=200±210 nm) when the 12 nm particles were present.

A similar 12 nm particle was observed when the NaCl concentration was increased to 2 M (FIGS. 23 and 24). At this NaCl concentration, the Tₜ was lowered to 17° C. as determined by the turbidity measurements. This temperature range was limited at lower temperatures by the condensation of water vapor on the sample cuvette. Therefore, between 20° C. and 30° C., the thioredoxin-ELP1 [V-20] had already transitioned into stable aggregates with average Rₕ of 2.4±1.7 μm. As the samples was heated beyond ˜36° C., the Rₕ of the aggregates gradually decreased in size to 230±170 nm and 12 nm particles (Rₕ=12±4.7 nm) appeared. The percentage of scattered light attributable to the 12 nm particles also gradually increased at the expense of the shrinking larger aggregates.

Thioredoxin-ELP1 [V-20] and thioredoxin-ELP1 [V₅A₂G₃-90] were each purified by ITC from the soluble fraction of lysed E. coli cultures, and thioredoxin(His₆) was purified by IMAC as a control having no ELP tag. Representative SDS-PAGE results for the purification of each protein are shown in FIG. 25 (showing only the first round of ITC for the two ELP fusion proteins).

Lane A shows a molecular weight marker, labeled in kDa. Lanes B-D show IMAC purification of free thioredoxin(His₆), and Lanes E-H and I-L show ITC purification of thioredoxin-ELP1 [V-20] and thioredoxin-ELP1 [V₅A₂G₃-90], respectively. Lanes B, E, and I are the soluble cell lysate. Lanes C and D are the IMAC column flow-through and elution product, respectively. For ITC purification, lanes F and J are the supernatant after inverse transition and centrifugation; lanes G and K are the pellet containing the target protein, after redissolving in PBS; and lanes H and L are the purified target protein thioredoxin, after cleavage with thrombin and separation from its ELP tag by a second round of ITC. The inverse transition was induced by the addition of 1.3 M NaCl, and the centrifugation was carried out at 33° C. The smaller ELP1 [V-20] tag was successfully used to purify the fusion protein by ITC to homogeneity, with a yield and purity similar to that of the free thioredoxin purified by a conventional affinity chromatography method.

Note that the ELP tag was not stained by Coomassie, and therefore only the thioredoxin portion of the fusion protein was visible in the stained gels. Qualitative comparison of the expression levels in the soluble cell lysate for thioredoxin-ELP1 [V-20] (lane E) and thioredoxin-ELP1 [V₅A₂G₃-90] (lane I) clearly showed that truncating the size of the ELP tag from 36 kDa to 9 kDa greatly enhanced the expression yield of the thioredoxin. Furthermore, FIG. 25 shows that thioredoxin-ELP1 [V-20] was expressed to a level qualitatively comparable to that of free thioredoxin (lane B). SDS-PAGE analysis also showed that there was no detectable loss to the insoluble fraction of the cell lysate for any the target proteins (results not shown).

For the ITC purifications, the ELP phase transition was triggered by adding 1.3 M additional NaCl and increasing the solution temperature to above ˜33° C. The cell lysates became turbid as a result of aggregation of the thioredoxin-ELP fusion proteins, which were then separated from solution by centrifugation at ˜33° C. to form a translucent pellet at the bottom of the centrifuge tube. SDS-PAGE showed that most contaminating E. coli proteins were retained in the decanted supernatant (FIG. 25, lanes F and J). The pellets were dissolved in PBS at ˜4° C., and centrifuged at low temperature (˜12° C.) to remove any remaining insoluble matter. The supernatants containing purified thioredoxin-ELP fusion proteins were retained (FIG. 25, lanes G and K). Finally, purified, free thioredoxin was obtained after cleavage of each fusion protein by thrombin at the encoded recognition site located between thioredoxin and the ELP tag, followed by a second round of ITC to remove the ELP tag from solution (FIG. 25, lanes H and L). Here, thrombin was retained with the target thioredoxin in the supernatant (although it was below the detection limit of Coomassie staining), however a thrombin-ELP fusion could be developed that would be removed after cleavage along with the free ELP.

These SDS-PAGE results clearly showed that thioredoxin can be purified by ITC to homogeneity, as ascertained by Coomassie staining, using the shorter, 9 kDa ELP 1 [V-20]. However, differences were observed in the purification efficiency of the two ELP fusion proteins under these conditions, as qualitatively ascertained by SDS-PAGE. Lanes I through K show that recovery of thioredoxin-ELP1 [V₅A₂G₃-90] by ITC from the soluble cell lysate was essentially complete, whereas lanes E though G show that a small but significant fraction of thioredoxin-ELP1 [V-20] remained in the discarded supernatant (lane G). The level of purity obtained by ITC with the ELP1 [V-20] tag was qualitatively as good or better than that obtained by IMAC purification of the free thioredoxin, although with IMAC purification there was no detectable loss of the target protein in the column flow-through (lane C).

Using UV-visible spectrophotometry, the yield of each protein recovered by ITC or IMAC purification was quantified (FIG. 26). Although these data described the amount of protein recovered after purification, the SDS-PAGE results in FIG. 25 suggested that this quantity was nearly equal to expression yield in the soluble lysate. For this analysis, four cultures were grown in parallel under identical conditions for each of the three protein constructs. For experimental convenience, these data were obtained for 50 ml cultures, and extrapolated to yield per liter of culture. Purification of separate 1 liter cultures confirmed that the actual yields closely matched the extrapolated values (data not shown).

FIG. 26 is a graph of purified protein yield. The total yields of the thioredoxin(His₆), thioredoxin-ELP1 [V-20], and thioredoxin-ELP1 [V₅A₂G₃-90] from the 50 ml test cultures are shown, extrapolated to milligrams per liter of culture (mean±SD, n=4). The separate contributions of the ELP tag and thioredoxin to the yield, as calculated using their respective mass fractions of the fusion protein, are also shown for comparison. With all other experimental conditions identical, reducing the ELP tag from 36 (thioredoxin-ELP1 [V₅A₂G₃-90]) to 9 kDa (thioredoxin-ELP1 [V-20]) resulted in a near four-fold increase in the yield of the target thioredoxin.

The data in FIG. 26 show that decreasing the molecular weight of the ELP tag can dramatically increase the yield of thioredoxin. Under experimentally identical conditions of E. coli culture, decreasing the ELP tag size from 36 kDa in thioredoxin-ELP1 [V₅A₂G₃-90] to 9 kDa in thioredoxin-ELP1 [V-20] increased the yield of fusion protein by 70% (82±12 mg/L versus 137+21 mg/L, respectively; P<0.005, unpaired t test). Furthermore, since truncating the size of the ELP tag reduced its mass fraction in the fusion protein, the target protein thioredoxin (i.e., if separated from the fusion protein at the thrombin cleavage site) constituted a larger fraction of the mass in the fusion protein yield. Thus, the yield of thioredoxin was 365% greater using the smaller tag (23±3.3 mg/L versus 83±12 mg/L for the larger and smaller tags, respectively; P<0.0001). This yield of thioredoxin obtained by ITC using the 9 kDa tag was statistically indistinguishable from that obtained for thioredoxin expressed without an ELP tag and purified using IMAC (93±13 mg/L; P>0.25).

These results corroborated the SDS-PAGE results since the relative yields of thioredoxin (FIG. 26) correlated with the expression levels observed in the cell lysate (FIG. 25). The yield of the ELP tag was the same for both fusion proteins (59±8.6 mg/L for thioredoxin-ELP1 [V₅A₂G₃-90] and 54±8.1 mg/L for thioredoxin-ELP1 [V-20]; P>0.4). This was consistent with previous observations that the gravimetric yield of the ELP tag in thioredoxin fusion proteins was essentially constant with respect to ELP molecular weight within the ELP1 [V₅A₂G₃-90]] family of polypeptides ranging from 24 to 72 kDa.

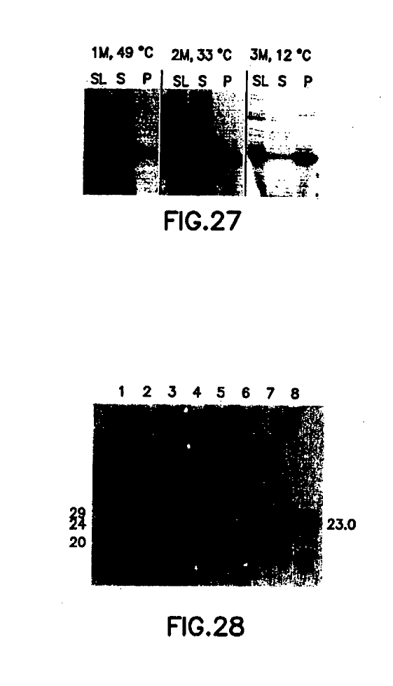

To demonstrate the relationship between purification efficiency and ITC solution conditions, we repeated ITC purification of the thioredoxin-ELP1 [V-20] fusion protein using different combinations of salt concentration and centrifugation temperature (FIG. 27).

FIG. 27 shows SDS-PAGE analysis of the effect of NaCl concentration and centrifugation temperature on purification of thioredoxin-ELP[V-20] by ITC: SL=soluble cell lysate; S=supernatant after inverse transition of fusion protein and centrifugation to remove aggregated target protein; and P=redissolved pellet containing the purified fusion protein, after dissolution in PBS. The molar NaCl concentration and centrifugation temperature for each purification is noted at top. Although a high level of purity was achieved in each case, selection of an appropriate NaCl concentration and centrifugation temperature is critical to achieve complete purification efficiency.

When PBS with 1 M NaCl combined with centrifugation at 49° C. was used for ITC purification, the majority of the target fusion protein was lost in the discarded supernatant (FIG. 27, left panel). When PBS plus 2 M NaCl and a centrifugation temperature of 33° C. was used (FIG. 27, center panel), more than half of the target protein was captured by centrifugation. Finally, using PBS with 3 M NaCl and centrifugation at 12° C. (FIG. 27, right panel), the vast majority of the target protein was successfully purified. Although the target protein was purified to homogeneity in each of these examples, these results showed that selection of salt concentration and temperature was an important factor influencing the efficiency of ITC purification.

The objective of the this example was to produce an ELP tag for ITC purification that was reduced in size relative to those previously reported, and to characterize the effect of this reduction on expression levels and on purification efficiency. In the previously reported effort, a first generation of ELP purification tags was developed based on a ELP1 [V₅A₂G₃-10] monomer sequence. This sequence was recursively oligomerized to create a library of synthetic genes encoding ELPs with molecular weights ranging from 4 kDa (ELP1 [V₅A₂G₃-10]) to 71 kDa (ELP1 [V₅A₂G₃-180]). This particular guest residue composition was selected based on previous studies of Urry et al., and ELPs with this composition were predicted to exhibit a Tₜ of 40° C. for molecular weights of ˜100 kDa in water. A 40° C. Tₜ was targeted so that the fusion proteins would remain soluble during culture at 37° C., but could be induced to reversibly aggregate through the ELP phase transition by a modest increase in salt concentration or solution temperature.

Although the Tₜ's of the higher molecular weight constructs approached 40° C. (Tₜ=42° C. for the thioredoxin-ELP1 [V₅A₂G₃-180], with MW_{ELP}=71 kDa, in PBS at 25 μM), the Tₜ of the thioredoxin-ELP1 [V₅A₂G₃] fusion proteins increased dramatically with decreasing molecular weight (T=77° C. for thioredoxin-ELP1 [V₅A₂G₃-30], with MWELP=13 kDa, under the same conditions). The high Tₜ's of the lower molecular weight ELPs required the addition of a very high concentration of NaCl (>3 M) to reduce their Tₜ to a useful temperature (e.g., 20-40° C.), which precluded their general use for purification by ITC because of the potential for salt-induced denaturation of target proteins. Although the larger ELP1 [V₅A₂G₃] polypeptides were successfully used to purify thioredoxin and second model target protein, tendamistat, we observed that the yield of the fusion protein was significantly decreased as the ELP1 [V₅A₂G₃] chain length was increased.

These observations motivated the redesign of the ELP expression tag in the above experiment to reduce the size of the ELP expression tag while also depressing its Tₜ, so that lower molecular weight ELP tags would exhibit a Tₜ near 40° C. at more moderate NaCl concentrations. The second monomer gene, which was newly synthesized for this study, encoded a five pentamer ELP sequence where the fourth guest residue was exclusively Val (ELP1 [V-5]). Because the Val present in ELP1 [V] was more hydrophobic than the Ala and Gly present in ELP1 [V₅A₂G₃], thioredoxin-ELP1 [V] fusion proteins were predicted to have a Tₜ of 40° C. at smaller ELP molecular weights than for thioredoxin-ELP1 [V₅A₂G₃] fusions.

The ELP1 [V-20] sequence (four tandem repeats of the ELP1 [V-5] gene) was selected from a library of ELP1 [V-5] oligomers for further characterization at a ITC purification tag due to the empirical observation of its Tₜ near 40° C. at lysate protein concentration with moderate (1 M) NaCl. In the present example, the thioredoxin-ELP1 [V-20] construct (MW_{ELP}=9 kDa) was compared to the previously described thioredoxin-ELP1 [V₅A₂G₃-90] construct (MW_{ELP}=36 kDa) because the two fusion proteins had very similar Tₜ's in lysate conditions for varying NaCl concentrations, as can be seen in FIG. 13. That is, they are thermal analogs from each of the two libraries that meet the above-described desired Tₜ characteristics for ITC purification tags.

Although previous observations suggested that decreasing the size of the ELP was likely to enhance the overall expression level of the fusion protein, it was not obvious, a priori, whether the decreased size of the tag would adversely affect purification of ELP fusion proteins by ITC. Therefore, in addition to its effect on the expression level of the target protein, the effect of the ELP tag length on the purification efficiency (i.e., degree of recovery) and on the purity of the target protein after ITC purification was explored.

The SDS-PAGE and spectrophotometry results (FIGS. 25-27) show that decreasing the ELP molecular weight from 36 kDa to 9 kDa enhanced expression of the fusion protein by nearly four-fold, and did not adversely affect the purity of the final protein under any of the solution conditions (i.e., NaCl concentration and temperature) used to induce the inverse transition. The level of expression with the ELP[V-20] tag was comparable to that of free thioredoxin, indicating that further reduction of the ELP tag would not be expected to increase the thioredoxin yield.

One possible explanation for the observed increase in thioredoxin yield as the ELP tag length was reduced is that, for given culture conditions, the mass of ELP that can be expressed by the cells is limited independent of ELP chain length. This is supported by the results in FIG. 26, as well as by observations with other ELPs of various molecular weight. Such a limitation would likely be engendered by a metabolic factor, perhaps by an insufficient tRNA pool and/or by amino acid depletion due to the highly repetitious ELP sequence. If the mass yield of ELP is a limiting factor, then this provides a rationale for the increased thioredoxin yields with the ELP[V-20] tag. For a given gravimetric yield of ELP, decreasing the ELP chain length increases the molar yield of the fusion protein, and hence, the target protein. Furthermore, this also suggests that increasing the gravimetric yield of ELP, e.g., through supplementation of specific, ELP-related amino acids during culture, offers another potential route for improvement of the fusion protein yield.

Although the yield of the target protein was increased with the shorter ELP1 [V-20] tag, this benefit entailed a more complicated transition behavior. The efficiency of recovery with this tag depends on the solution conditions used for ITC (FIG. 27), whereas, with the larger ELP1 [V₅A₂G₃-90] tag, recovery of the fusion protein was complete under all solution conditions (results not shown). Thus, although the truncated ELP1 [V-20] tag enabled thioredoxin to be purified to homogeneity by ITC, the efficiency of purification was sensitive to the specific conditions chosen to induce the inverse transition.

The turbidity and DLS data (FIGS. 13-24) provide insights into the sensitivity of purification efficiency for the smaller ELP1 [V-20] tag on solution conditions. While solutions of thioredoxin-ELP1 [V₅A₂G₃-90] remained turbid at all temperatures above Tₜ, the turbidity profiles for thioredoxin-ELP1 [V-20], after an initial rapid rise at Tₜ, began to clear with further heating at a temperature above Tₜ. This phenomenon of clearing with increasing temperature has not been previously observed, to my knowledge, with other ELPs or ELP fusion proteins. To study this complex aggregation behavior, the sizes of the fusion protein particles were measured using dynamic light scattering as a function of temperature to determine the structural basis for the markedly different turbidity profiles of the two fusion proteins.

With increasing temperature, monomers of thioredoxin-ELP1 [V₅A₂G₃-90] went through an abrupt, discontinuous phase transition to form aggregates that persisted at all temperatures above Tₜ with a steady state Rₕ of 2 μm. Because the aggregates were stable above the Tₜ, the aggregated protein was able to be completely recovered by centrifugation at any temperature above its Tₜ (or at any NaCl concentration for which the Tₜ was depressed to below the solution temperature).

Although thioredoxin-ELP1 [V-20] also exhibited an abrupt phase transition to form aggregates, these aggregates were not stable at all temperatures above its phase transition. As the temperature was increased beyond the Tₜ, small aggregates with Rₕ of ˜12 nm formed at the expense of mass in the larger aggregates, which also showed a decrease in size with increasing temperature. This provides a structural rationale for the decrease in turbidity observed above the Tₜ of thioredoxin-ELP1 [V-20]. Upon heating to temperatures greater than Tₜ (beginning ˜10° C. above Tₜ for PBS with 1 M NaCl, and ˜115° C. above Tₜ for PBS with 2 M NaCl), larger scattering centers were converted to small particles that scatter light less effectively. The formation of these 12 nm particles at the expense of the larger aggregates resulted in incomplete recovery by centrifugation of the fusion protein from the soluble lysate. Thus, when ELP1 [V-20] (and potentially other small ELP tags) were used for purification of fusion proteins, it was imperative for complete protein recovery that a NaCl concentration and complimentary solution temperature be chosen such that only the larger aggregates, which are easily separable by centrifugation, were present in suspension.

On the basis of size alone, the precise structure of the 12 nm particle was not able to be predicted. However, the particle may be a micelle-like structure containing a small number of fusion protein molecules (perhaps on the order of 40 to 60) that are aggregated such that solvated thioredoxin domains encase the collapsed, hydrophobic ELP domains in the particle's core. The size of the observed particle (Rₕ≈12 nm) would be consistent with such a structure, as the hydrophilic thioredoxin “head” was ˜3 nm in diameter and the hydrophobic 20 pentamer ELP “tail” was ˜7 nm in length.

The proximity of the thioredoxin molecules required in such a micellular structure may also explain the irreversible aggregation that is observed at temperatures greater than ˜55° C. Denaturation at this low temperature was only observed for thioreoxin fused to ELP1 [V-20], and only for NaCl concentrations of 1 M and greater. And, it is only for these conditions that the 12 nm particle was observed. An extremely high effective concentration of thioredoxin in the solvated, hydrophilic shell of the micelle, with little ELP buffering between thioredoxin molecules, is consistent with the observed decrease in thermal stability.

The examples in FIG. 27 illustrate the importance of appropriate selection of NaCl concentration and solution temperature during ITC. The three centrifugation temperatures were selected for experimental convenience: 12° C. when a microcentrifuge was placed in a 4° C. refrigerated laboratory cabinet, 33° C. when placed on a laboratory bench top at 22° C., and 49° C. when placed in a 37° C. static incubator (all sample temperatures were measured directly by thermocouple after a 10 minute centrifugation). The NaCl concentrations were selected in 1 M increments to depress the Tₜ to some point below each centrifugation temperature.

For the first two examples (FIG. 27, left and center), recovery was incomplete because at these combinations of centrifugation temperature and NaCl concentration, thioredoxin-ELP1 [V-20] showed a two phase behavior where larger aggregates coexisted with the 12 nm particles. Because of their small mass, these particles remained suspended during centrifugation, and only the fraction of fusion protein contained in the larger aggregate phase was removed by centrifugation and recovered in the resolubilized pellet. At 49° C., the thioredoxin-ELP1 [V-20] turbidity profile in PBS with 1 M NaCl was significantly decreased from its maximum value (FIG. 13), and data showed that a majority of the scattering intensity came from the 12 nm particles (FIGS. 21 and 22). Correspondingly, the SDS-PAGE data in FIG. 27 shows that only a small fraction of the fusion protein present was captured by centrifugation during ITC purification. At 33° C. in PBS with 2 M NaCl, although still below its maximum value, the turbidity of thioredoxin-ELP1 [V-20] was closer to its peak value (FIG. 13), and the data shows that the scattering intensity attributed to the 12 nm particle was much smaller (FIGS. 23 and 24). Consistent with these observations, a majority of fusion protein was captured by ITC purification as ascertained by SDS-PAGE in FIG. 25, although loss in the supernatant due to the 12 nm particles was still significant.

Using a centrifugation temperature of 12° C. in PBS with 3 M NaCl, recovery of the fusion protein in the resolubilized pellet was nearly complete (FIG. 27, right). Under these conditions, the solution turbidity was very near its maximum value (FIG. 13). The degree of turbidity, combined with the trends in particle size distribution established for lower salt concentrations in FIGS. 21-24, suggest that the complete recovery obtained by ITC with these conditions is explained by the presence of only the larger aggregates for these solution conditions.

These examples illustrate that for efficient ITC purification of thioredoxin-ELP1 [V-20], and potentially for other soluble fusion proteins with small ELP tags, the NaCl concentration and centrifugation temperature should be selected to achieve the maximum point in the turbidity profile. For microcentrifuges without temperature control, this is most practically achieved by determining the centrifuge sample temperature, and then adjusting the Tₜ of the fusion protein by the precise addition of salt. For larger centrifuges that are equipped with refrigeration systems, recovery efficiency can be maximized by the combined alteration of NaCl concentration and centrifugation temperature. The required precision in controlling solution conditions during ITC for thioredoxin-ELP1 [V-20] versus that for thioredoxin-ELP1 [V₅A₂G₃-90], which can be fully recovered using any combination of temperature and salt concentration that induces the inverse transition, is the price paid for the four-fold increase in yield of the target protein.

Decreasing the length of the ELP purification tag from 36 to 9 kDa produced a four-fold increase in the expression levels of E. coli thioredoxin, a model target protein. The expression level with the 9 kDa tag was similar to that of free thioredoxin expressed without an ELP tag, and therefore further reduction of the ELP tag size is not likely to provide any additional benefit. Although truncation of the ELP did not adversely affect the purity of the final protein product, it is important to select an appropriate combination of salt concentration and solution temperature to favor the formation of larger aggregates during ITC purification.

### Example 2

### High-Throughput Purification of Recombinant Proteins Using ELP Tags

The gene for the 5-polypentapeptide VPGVG ELP sequence was constructed by annealing two 5′-phosphorylated synthetic oligonucleotides (Integrated DNA Technologies, Coralville, Iowa) to yield double stranded DNA with PflMI and HinDIII compatible ends. This gene was inserted into a PflMI/HinDIII linearized and dephosphorylated modified pUC-19 (New England Biolabs, Beverly, Mass.) vector and polymerized using recursive directional ligation with PflMI and Bgll (Meyer, 1999; Meyer, 2000) to generate the gene for the 20-polypentapeptide ELP sequence. This ELP gene was then excised with PflMI and Bgll, gel purified (QIAquick Gel Extraction Kit, Qiagen, Valencia, Calif.), and inserted into a SfiI linearized and dephosphorylated modified pET32b vector (Novagen, Madison, Wis.; Meyer, 1999). This expression vector was then transformed into the BLR(DE3) (Novagen) E. Coli expression strain.

The aforementioned cells were taken from frozen (DMSO) stock and streaked onto agar plates supplanted with 100 μg/ml ampicillin and allowed to grow overnight. Two hundred microliters of growth media (100 μg/ml ampicillin in CircleGrow media; Qbiogene, Inc., Carlsbad, Calif.) were injected into each well of a standard 96 well microplate (Costar, Corning Inc., Corning, N.Y.) using a multichannel pipetter. Using 200 μl pipet tips, each well of the microplate was inoculated with a pinhead-sized aggregation of cells from colonies on the aforementioned agar plates. With the lid on, the microplate was incubated at 37° C. and shaken at 275 r.p.m. The microplate was held in place in the shaker using an ad hoc microplate holder. The cultures were induced by adding isopropyl α-thiogalactopyranoside to a final concentration of 1 mM when the OD₆₅₀ reached 0.65 for a majority of the cultures as measured using a microplate reader (Thermomax; Molecular Devices Co., Sunnyvale, Calif.)—this optical density corresponds to an OD₆₅₀ of 2.0 as measured using an UV-visible spectrophotometer (UV-1601, Shimadzu Scientific Instruments, Inc.). The cultures were incubated and shaken for 4 hours post-induction and then harvested by centrifugation at 1100 g for 40 minutes at 4° C. using matched-weight microplate carrier adaptors (Beckman Instruments, Inc., Palo Alto, Calif.). The media was discarded and the cell pellets were frozen in the microplates at −80° C. until they were ready to be purified.

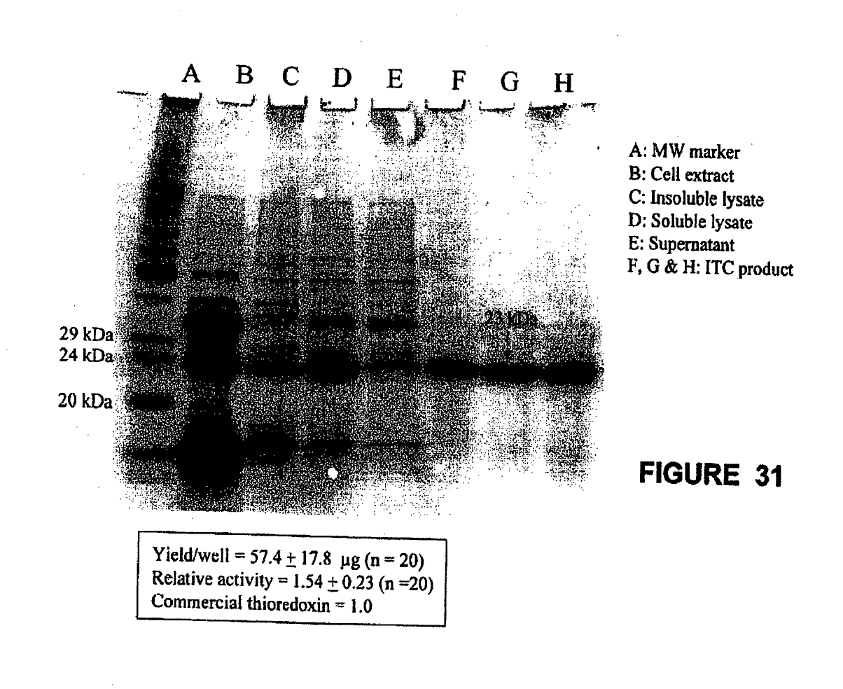

The ELP1 [V-20]/thioredoxin protein was purified from cell cultures in the microplates as follows. The cells were lysed by adding 1 μl of lysozyme solution (25 mg/ml; Grade VI; Sigma, St. Louis, Mo.) and 25 ul of lysis buffer (50 mM NaCl, 5% glycerol, 50 mM Tris-HCl, pH 7.5) to each well. The micro plate was then shaken using an orbital shaker at 4° C. for 20 minutes. Two μl of 1.35% (by mass) sodium doxycholate solution were added to each well and the microplate was shaken at 4° C. for 5 minutes. Two μl of deoxyribonuclease I solution (100 units/ul; Type II; Sigma, St. Louis, Mo.) were added to each well and the microplate was shaken at 4° C. for 10 minutes. The microplate was then centrifuged at 1100 g for 20 minutes at 4° C. using matched-weight microplate carrier adaptors (Beckman Instruments, Inc., Palo Alto, Calif.) to pellet cell particulates and insoluble proteins. Two μl of 10% (by mass) polyethylenimine solution was added to each well and the microplate was shaken at 4° C. for 15 minutes. The microplate was then centrifuged at 1100 g for 20 minutes at 4° C. to pellet DNA. The supernatants were transferred to wells on a new microplate and the old microplate was discarded. To induce ELP1 [V-20]/thioredoxin aggregation, 20 μl of saturated NaCl solution was added to each well; a marked increase in turbidity indicated aggregation of the target protein. To pellet the aggregated proteins, the microplate was centrifuged at 1100 g for 40 minutes at 30° C. The protein pellets were resolubilized in 30 μl of phosphate buffer solution after which the microplate was centrifuged at 1100 g for 20 minutes at 4° C. to remove insoluble lipids. Finally, the purified protein supernatents were transferred to wells of a new microplate and stored at 4° C. SDS-PAGE gel analysis for the ELP1 [V-20]/thioredoxin fusion protein purified by ITC is shown in FIG. 31.

Alternatively, ELPs/ELP-fusion proteins can be purified using a commercially available extraction reagent in accordance with the following protocol. Lyse cells by adding 25 microliters of Novagen BugBuster Protein Extraction Reagent to each microplate well. The microplate is placed on a Fisher Vortex Genie at shaker speed 2 (alternatively on an orbital shaker at maximum speed) for fifteen minutes at room temperature. Using the microplate adaptors, centrifugation is conducted (2300 rpm, 1700×g for Beckman adaptor for the JS4.2 rotor) for 20 minutes at 4 degrees Celsius to form a pellet. Add 2 microliters polyethylenimine (to 0.66%) to the wells and shake using Vortex Genie or shaker for 5 minutes. Incubate on ice 10 minutes, shaking occasionally. Using the microplate adaptors, centrifuge at maximum speed for 25 minutes at 4 degrees Celsius. Transfer the supernatant to the new microplate and discard the old microplate with the pellet. Add NaCl (crystals) and/or increase the solution temperature to induce ELP aggregation. Mix by shaking only—pipeting will aggregate the ELP on the pipet tip. Solution should turn turbid to some extent. Centrifuge at a temperature above the transition temperature (2300 rpm, 1700 g, 35-40 degrees Celsius, 45 minutes). Discard supernatant and resuspend the pellet (typically non-visible or a tiny pellet) in 30 microliters of cold buffer of choice (PBS) by repeatedly pipeting around the bottom and walls of the well. Centrifuge (2300 rpm, 1700×g, 4 degrees Celsius, 20 minutes) to spin out insoluble impurities such as lipids. Transfer the supernatant to another microplate. The purified ELP may be stored frozen at −80 degrees Celsius in the microplate until ready for use. (For fusions, ensure that freezing is suitable for the fusion protein.) The appropriate NaCl concentration and temperature employed in this technique depends on the ELP, fusion partner, and ELP concentration. The objective is to lower the effective ELP transition temperature at least 3 to 5 degrees below the solution temperature. An effective transition temperature of 25-30 degrees Celsius and warm centrifugation at 35-40 degrees Celsius has been usefully employed, although higher temperatures may be used if tolerated by the fusion protein.

Protein concentration was determined by measuring A₂₈₀ (UV-1601, Shimadzu Scientific Instruments, Inc.) and using the molar extinction coefficient for ELP1 [V-20]/Thioredoxin (ε=19,870); this assumes that the ELP1 [V-20]/Thioredoxin protein samples are pure of protein and DNA impurities. Thioredoxin activity was determined using an insulin reduction assay (Holmgren, 1984).

For the construction of the fusion protein, a small ELP tag was designed with a Tₜ of around 70° C., using previously published theoretical Tₜ data (Urry, 1991). Characterization of the ELP tag showed that the Tₜ was 76.2° C., confirming that it is possible to rationally design ELP tags with specified Tₜ. For the ELP/thioredoxin fusion protein, the Tₜ in low salt buffer, 1 M, and 2 M salt solutions were 68° C., 37° C. and 18° C., respectively, confirming that fusion of a soluble protein to an ELP tag minimally affects its Tₜ and showing that the Tₜ can be manipulated over a wide range by adjusting the salt concentration.

Based on the foregoing, the creation of a family of plasmid expression vectors that contain an ELP sequence and a polylinker region (into which the target protein is inserted) joined by a cleavage site can be employed to facilitate the expression of a variety of proteins. The ELP sequences embedded in such family of plasmids can have different transition temperatures (by varying the identity of the guest residue). The expression vector for a particular target protein is desirably selected based on the protein's surface hydrophobicity characteristics. The salt concentration of the solution then is adjusted during purification to obtain the desired Tₜ.

For protein expression involving growth of cell cultures in microplate wells, the cell cultures can be desirably induced at OD₆₀₀≈2 and grown for 4 hours post-induction. The cell density at induction for the microplate growths is two to three times that achieved by conventional protein expression protocols. Even at these high cell densities, rapid and healthy cell growth can be maintained in the microplate wells by aeration of the cultures, which as grown in the wells are characterized by a high surface area to volume ratio. Cell cultures that are grown longer post-induction yielded minimally more target protein, and growth using a hyper expression protocol (Guda, 1995) had much more contaiminant protein (around tenfold) with minimally more fusion protein. In order to avoid evaporation of the cell media in the high surface area to volume ratio cell growth in the microplate wells, it was necessary to cover the microplate with an appropriate lid during growth and to infuse the cell growth with additional media during induction. On a per liter basis, cultures grown in the microplate wells had a higher level of fusion protein expression than cultures grown with conventional protocols.

High throughput protein purification utilizing ITC was successful when cells were lysed with commercial nonionic protein extraction formulations. After cell lysis, addition of polyethylenimine removed nucleic acids and high molecular mass proteins from the soluble fraction of the crude lysate upon centrifugation. At the fusion protein and salt concentrations of the soluble lysate, the Tₜ of the fusion protein was approximately 65° C. Heating the soluble lysate above this temperature to induce fusion protein aggregation denatures and precipitates soluble contaminant proteins as well as the target protein itself. Furthermore, this temperature could not be maintained within the centrifuge chamber during centrifugation. Therefore, salt was added to the soluble lysate to approximately 2 M; this depressed the Tₜ of the fusion protein to approximately 18° C., allowing for aggregation of the fusion protein at room temperature. This salt concentration did not precipitate any contaminant proteins nor did it alter the functionality of the final purified protein product.

High throughput protein purification using ITC was both effective and efficient. About 15% of the expressed fusion protein was lost in the insoluble protein fraction of the cell lysate. Centrifugation of the sample after fusion protein aggregation effectively separated the proteins: 90% of the fusion protein was pelleted while 10% of the fusion protein remained in the supernatant along with all soluble contaminant proteins. Overall, about 75% of the expressed protein was abstracted using ITC purification and E. Coli contaminant protein levels in the purified products were below those detectable by SDS-PAGE. The purification process can be expedited and purification efficiency increased by increasing the centrifugation speeds; higher centrifugation speeds allow for reduced centrifugation times and at higher centrifugation speeds (5000 g), all of the fusion protein is pelleted during centrifugation post aggregation. Addition of thrombin completely cleaved the fusion protein and a second round of ITC separated the ELP tag from the thioredoxin target protein with no loss of thioredoxin.

The average amount of fusion protein purified per well determined using absorbance measurements (A₂₈₀, ε=19,870) was 33 ug with a standard deviation of 8.5 ug. Values were dispersed evenly between 19.7 and 48.3 ug per well. The large variation in yield of purified protein was due more to the different amounts of protein expressed in the different wells than to a variation in the purification efficiency of the ITC process. Varying amounts of protein were expressed in the different cell cultures because 1) the imprecision of the inoculation meant that cell cultures had varying amounts of cells to begin with and 2) due in all likelihood to more abundant aeration, the cell cultures in peripheral wells tended to have faster growth and reach a higher stationary phase cell density. For simplicity of effort, all of the cell cultures were induced and then harvested at the same times as opposed to induction and harvesting of individual cell cultures.

The enzymatic activity of the thioredoxin target protein was measured using an insulin reduction assay. The average amount of fusion protein per well, determined on the basis of such enzymatic activity, was 35.7 ug with a standard deviation of 8.0 ug. Again, values were dispersed evenly, between a minimum of 24.6 and a maximum of 50.8 ug per well. It is important to note that thioredoxin was enzymatically active though still attached to the ELP tag. The thioredoxin expressed and purified using this high throughput ITC technique had, on average, 10.3% greater enzymatic activity per unit mass than that of commercial thioredoxin (Sigma), a testament to the gentleness of and purity achieved by the ITC process.

On average, high throughput ELP/thioredoxin protein expression and purification produced around 160 mg of protein per liter of growth. This is comparable to ELP/thioredoxin yields obtained using conventional protein expression and ITC purification methods (140-200 mg protein/L of growth).

FIG. 28 is an SDS-PAGE gel of the stages of high throughput protein purification using microplates and inverse transition cycling according to the above-described procedure, in which ELP/thioredoxin fusion protein was purified (Lane 1: molecular mass markers (kDa) (Sigma, wideband; Lane 2: crude lysate; Lane 3: insoluble proteins; Lane 4: soluble lysate; Lane 5: supernatant containing contaminant proteins; Lane 6: purified ELP/thioredoxin fusion protein; and Lanes 7 and 8: purified ELP/thioredoxin fusion proteins from other wells). The ELP/thioredoxin fusion protein was purified using the documented protocol. Gel samples were denatured with SDS, reduced with beta-mercaptoethanol, and run at 200 V for 45 minutes on a 10-20% gradient Tris-HCl gel.

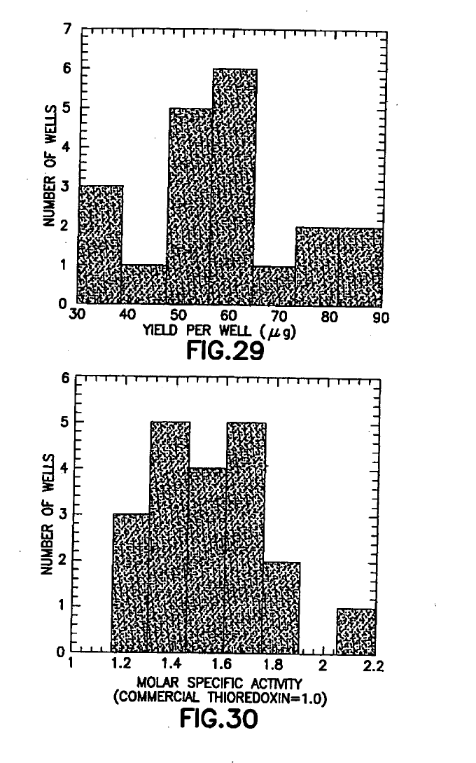

FIGS. 29-30 show histograms for quantitization of purified protein samples. FIG. 29 is a histogram of total fusion protein per well as determined using absorbance measurements (A₂₈₀, ε=19,870) (n=20, >=32.97, σ=8.48). FIG. 30 is a histogram of fusion protein functionality/purity for each sample compared to commercial thioredoxin (from Sigma) (n=20, μ=1 10.37%, σ=16.54%).

Considering the high throughput protein expression and purification method of the invention, it is noted that whereas nickel-chelated multiwell plates can purify only 1 ng of His-tagged protein per well, the capacity of high throughput purification using ITC is limited only by the amount of the protein that can expressed by cultures grown in the well; for ELP tagged proteins, the level of protein expression is in the tens of microgram range.

High throughput purification using ITC thus provides high yields, producing sufficient protein for multiple assays and analyses. Milligram levels of purified protein can be obtained by growing cell cultures in other vessels and transferring the resuspended cell pellet to the multiwell plate for the purification process. Finally, such high throughput purification technique is technically simpler and less expensive than current conventional commercial high throughput purification methods as it requires only one transfer of purification intermediates to a new multiwell plate.

### Example 3

### Construction of Various ELP Gene Expression Series

Bacterial Strains and Plasmids: Cloning steps were conducted in Escherichia coli strain XL I-Blue (recA1, endA1, gyrA96, thi-1, hsdr17 (rₖ⁻, mₖ⁺), supE44, relA1, lac[F′, proAB, lacI^{q}ZΔM15, Tn10 (Tet^{r})] (Stratagene La Jolla, Calif.). pUC19 (NEB, Beverly, Mass.) was used as the cloning vector the ELP construction (Meyer and Chilkoti, 1999). Modified forms of pET15b and pET24d vectors (Novagen) were used to express ELP and ELP-fusion proteins in BL21 Star (DE3) strain (F⁻, ompT, hsdS_{B} (r_{B}⁻m_{B}⁻), gal, dcm, rne 131, (DE3)) (Invitrogen Carlsbed, Calif.) or BLR(DE3) (F⁻, ompT, hsdS_{B} (r_{B}⁻m_{B}⁻), gal, dCm, Δ(srl-recA) 306::Tn10(Tc^{R})(DE3)) (Novagen Madison, Wis.). Synthetic DNA oligos were purchased from Integrated DNA Technologies, Coralville, Iowa. All vector constructs were made using standard molecular biology protocols (Ausubel, et al., 1995).

Construction of ELP1 [V₅A₂G₃] Gene Series

The ELP1 [V₅A₂G₃] series designate polypeptides containing multiple repeating units of the pentapeptide VPGXG, where X is valine, alanine, and glycine at a relative ratio of 5:2:3.

The ELP1 [V₅A₂G₃] series monomer, ELP1 [V₅A₂G₃-10], was created by annealing four 5′ phosphorylated, PAGE purified synthetic oligos to form double stranded DNA with EcoR1 and HindIII compatible ends (Meyer and Chilkoti, 1999). The oligos were annealed in a 1 μM mixture of the four oligos in 50 μl 1× ligase buffer (Invitrogen) to 95° C. in a heating block than the block was allowed to cool slowly to room temperature. The ELP1 [V₅A₂G₃-10]/EcoR1-HindIII DNA segment was ligated into a pUC19 vector digested with EcoR1 and HindIII and CIAP dephosphorylated (Invitrogen) to form pUC19-ELP1[V₅A₂G₃-10]. Building of the ELP1 [V₅A₂G₃] series library began by inserting ELP1 [V₅A₂G₃-10] PflM1/Bgl1 fragment from pUC19-ELP1 [V₅A₂G₃-10] into pUC19-ELP1[V₅A₂G₃-10] linearized with PflM1 and dephosphorylated with CIAP to create pUC19-ELP1[V₅A₂G₃-20]. pUC19-ELP1[V₅A₂G₃-20] was then built up to pUC19-ELP1[V₅A₂G₃-30] and pUC19-ELP1[V₅A₂G₃-40] by ligating ELP1[V₅A₂G₃-10] or ELP1 [V₅A₂G₃-20] PflM1/Bgl1 fragments respectively into PflM1 digested pUC19-ELP1 [V₅A₂G₃-20]. This procedure was used to expand the ELP1 [V₅A₂G₃] series to create pUC19-ELP1 [V₅A₂G₃-60], pUC19-ELP1 [V₅A₂G₃-90] and pUC19-ELP1 [V₅A₂G₃-180] genes.

Construction of ELP1 [K₁V₁F₁] Gene Series

The ELP1 [K₁V₂F] series designate polypeptides containing multiple repeating units of the pentapeptide VPGXG, where X is lysine, valine, and phenylalanine at a relative ratio of 1:2:1.

The ELP1 [K₁V₂F,] series monomer, ELP1 [K₁V₂F₁-4] (SEQ ID NO: 18), was created by annealing two 5′ phosphorylated, PAGE purified synthetic oligos to form double stranded DNA with EcoRI and HindIII compatible ends (Meyer and Chilkoti, 1999). The oligos were annealed in a 1 μM mixture of the four oligos in 50 μl 1× ligase buffer (Invitrogen) to 95° C. in a heating block than the block was allowed to cool slowly to room temperature. The ELP1 [K₁V₂F₁-4]/EcoR1-HindIII DNA segment was ligated into a pUC19 vector digested with EcoR1 and HindIII and CIAP dephosphorylated (Invitrogen) to form pUC19-ELP1 [K₁V₂F₁-4]. Building of the ELP1 [K₁V₂F,] series library began by inserting ELP1 [K₁V₂F₁-4] PflM1/Bgl1 fragment from pUC19-ELP1 [K₁V₂F₁-4] into pUC19-ELP1 [K₁V₂F₁-4] linearized with PflM1 and dephosphorylated with CIAP to create pUC19-ELP1 [K₁V₂F₁-8]. Using the same procedure the ELP1 [K₁V₂F,] series was doubled at each ligation to form pUC19-ELP1[K₁V₂F₁-16], pUC19-ELP1[K₁V₂F₁-32], pUC19-ELP1 [K₁V₂F₁-64] and pUC19-ELP1 [K₁V₂F₁-128].

Construction of ELP1 [K₁V₇F₁] Gene Series

The ELP1 [K₁V₇F,] series designate polypeptides containing multiple repeating units of the pentapeptide VPGXG, where X is lysine, valine, and phenylalanine at a relative ratio of 1:7:1.

The ELP1 [K₁V₇F,] series monomer, ELP1 [K₁V₇F₁-9] (SEQ ID NO: 19), was created by annealing four 5′ phosphorylated, PAGE purified synthetic oligos to form double stranded DNA with PflMI and HindIII compatible ends. The ELP1 [K₁V₇F₁-9] DNA segment was than ligated into PflMI /HindIII dephosphorylated pUC19-ELP [V₅A₂G₃-180] vector thereby substituting ELP1 [V₅A₂G₃-180] for ELP1 [K₁V₇F₁-9] to create the pUC19-ELP1 [K₁V₇F₁-9] monomer. The ELP1 [K₁V₇F,] series was expanded in the same manor as the ELP1 [K₁V₂F₁] series to create pUC19-ELP1 [K₁V₇F₁-18], pUC19-ELP1 [K₁V₇F₁-36], pUC19-ELP1 [K₁V₇F₁-72] and pUC19-ELP1 [K₁V₇F₁-144].

Construction of ELP1 [V] Gene Series

The ELP1 [V] series designate polypeptides containing multiple repeating units of the pentapeptide VPGXG, where X is exclusively valine.

The ELP1 [V] series monomer, ELP1 [V-5] (SEQ ID NO: 14), was created by annealing two 5′ phosphorylated, PAGE purified synthetic oligos to form double stranded DNA with EcoRI and HindIII compatible ends. The ELP1 [V-5] DNA segment was than ligated into EcoRI/HindIII dephosphorylated pUC19 vector to create the pUC19-ELP1 [V-5] monomer. The ELP1 [V] series was created in the same manor as the ELP1 [V5A₂G₃] series, ultimately expanding pUC19-ELP1 [V-5] to pUC19-ELP1 [V-60] and pUC19-ELP1 [V-120].

Construction of ELP2 Gene Series

The ELP2 series designate polypeptides containing multiple repeating units of the pentapeptide AVGVP.

The ELP2 series monomer, ELP2 [5] (SEQ ID NO: 20), was created by annealing two 5′ phosphorylated, PAGE purified synthetic oligos to form double stranded DNA with EcoRI and HindIII compatible ends. The ELP2 [5] DNA segment was than ligated into EcoRI/HindIII dephosphorylated pUC19 vector to create the pUC19-ELP2[5] monomer. The ELP2 series was expanded in the same manor as the ELP1 [K₁V₂F₁] series to create pUC19-ELP2[10], pUC19-ELP2[30], pUC19-ELP2[60] and pUC19-ELP2[120].

Construction of ELP3 [V] Gene Series

The ELP3 [V] series designate polypeptides containing multiple repeating units of the pentapeptide IPGXG, where X is exclusively valine.

The ELP3 [V] series monomer, ELP3 [V-5] (SEQ ID NO: 21), was created by annealing two 5′ phosphorylated, PAGE purified synthetic oligos to form double stranded DNA with PfLM1 amino terminal and GGC carboxyl terminal compatible ends due to the lack of a convenient carboxyl terminal restriction site but still enable seamless addition of the monomer. The ELP3 [V-5] DNA segment was then ligated into PflM1/BglI dephosphorylated pUC19-ELP4[V-5], thereby substituting ELP4 [V-5] for ELP3 [V-5] to create the pUC19-ELP3[V-5] monomer. The ELP3 [V] series was expanded by ligating the annealed ELP3 oligos into pUC19-ELP3[V-5] digested with PflM1. Each ligation expands the ELP3 [V] series by S to create ELP3 [V-10], ELP3 [V-15], etc.

Construction of the ELP4 [V] Gene Series

The ELP4 [V] series designate polypeptides containing multiple repeating units of the pentapeptide LPGXG, where X is exclusively valine.

The ELP4 [V] series monomer, ELP4 [V-5] (SEQ ID NO: 22), was created by annealing two 5′ phosphorylated, PAGE purified synthetic oligos to form double stranded DNA with EcoRI and HindIII compatible ends. The ELP4 [V-5] DNA segment was than ligated into EcoRI/HindIII dephosphorylated pUC19 vector to create the pUC19-ELP4[V-5] monomer. The ELP4 [V] series was expanded in the same manor as the ELP1 [K₁V₂F₁] series to create pUC19-ELP4[V-10], pUC19-ELP4[V-30], pUC19-ELP4[V-60] and pUC19-ELP4[V-120].

The ELP genes were also inserted into other vectors such as pET15b-SD0, pET15b-SD3, pET15b-SD5, pET15b-SD6, and pET24d-SD21. The pET vector series are available from Novagen, San Diego, Calif.

The pET15b-SD0 vector was formed by modifying the pET15b vector using SD0 double-stranded DNA segment containing the multicloning restriction site (Sac1-Nde1-Nco1-Xho1-SnaB1-BamH1). The SD0 double-stranded DNA segment had xba1 and BamH1 compatible ends and was ligated intoXba1/BamH1 linearized and 5′-dephosphorylated pET15b to form the petl5b-SD0 vector.

The pET15b-SD3 vector was formed by modifying the pET15b-SD0 vector using SD3 double-stranded DNA segment containing a Sfi1 restriction site upstream of a hinge region-thrombin cleavage site followed by the multicloning site (Nde1-Nco1-Xho1-SnaB1-BamH1). The SD3 double-stranded DNA segment had Sac1 and Nde1 compatible ends and was ligated into Sac1/Nde1 linearized and 5′-dephosphorylated pET15b-SD0 to form the pET15b-SD3 vector.

The pET15b-SD5 vector was formed by modifying the pET15b-SD3 vector using the SD5 double-stranded DNA segment containing a Sfi1 restriction site upstream of a thrombin cleavage site followed by a hinge and the multicloning site (Nde1-Nco1-Aho1-SnaB1-BamH1). The SD5 double-stranded DNA segment had Sfi1 and Nde1 compatible ends and was ligated into Sfi1/Nde1 linearized and 5′-dephosphorylated pET15b-SD3 to form the pET15b-SD5 vector.

The pET15b-SD6 vector was formed by modifying the pET15b-SD3 vector using the SD6 double-stranded DNA segment containing a Sfi1 restriction site upstream of a linker region-TEV cleavage site followed by the multicloning site (Nde1-Nco1-Xho1-SnaB1-BamH1). The SD6 double-stranded DNA segment had Sfi1 and Nde1 compatible ends and was ligated into Sfi1/Nde1 linearized and 5′-dephosphorylated pET1 5b-SD3 to form the pET1 5b-SD6 vector.

The pET24d-SD21 vector was formed by modifying the pET24d vector using the SD21 double-stranded DNA segment with Nco1 and Nhe1 compatible ends. The SD21 double-stranded DNA segment was ligated into Nco1/Nhe1 linearized and 5′ dephosphorylated pET24d to create the pET24d-SD21 vector, which contained a new multi-cloning site Nco1-Sfi1-Nhe1-BamHI-EcoR1-SacI-SalI-HindIII-NotI-XhoI with two stop codons directly after the SfiI site for insertion and expression of ELP with the minimum number of extra amino acids.

The pUC19-ELP1 [V₅A₂G₃-60], pUC19-ELP1 [V₅A₂G₃-90], and pUC19-ELP1 [V₅A₂G₃-180] plasmids produced in XL1-Blue were digested with PflM1 and Bgl1, and the ELP-containing fragments were ligated into the Sfi1 site of the pET15b-SD3 expression vector as described hereinabove to create pET15b-SD3-ELP1[V₅A₂G₃-60], pET15b-SD5-ELP1[V₅A₂G₃-90] and pET15b-SD5- ELP1 [V₅A₂G₃-180], respectively.

The pUC19-ELP1 [V₅A₂G₃-90], pUC19-ELP1 [V₅A₂G₃-180], pUC19-ELP1 [V-60] and pUC19-ELP1 [V-120] plasmids produced in XL1-Blue were digested with PflM1 and Bgl1, and the ELP-containing fragments were ligated into the Sfi1 site of the pET15b-SD5 expression vector as described hereinabove to create pET15b-SD5-ELP1[V₅A₂G₃-90], pET15b-SD5-ELP1[V₅A₂G₃-180], pET15b-SD5-ELP1[V-60] and pET15b-SD5-ELP1[V-120], respectively.

The pUC19-ELP1 [V₅A₂G₃-90] plasmid produced in XL1-Blue was digested with PflM1 and Bgl1, and the ELP-containing fragment was ligated into the Sfi1 site of the pET15b-SD6 expression vector as described hereinabove to create pET15b-SD6-ELP1 [V₅A₂G₃-90].

The pUC19-ELP1[K₁V₂F₁-64], and pUC19-ELP1[K₁V₂F₁-128] plasmids produced in XL1-Blue were digested with PflM1 and Bgl1, and the ELP-containing fragments were ligated into the Sfi1 site of the pET24d-SD21 expression vector as described hereinabove to create pET24d-SD21-ELP1 [K₁V₂F₁-64] and pET24d-SD21-ELP1[K₁V₂F₁-128], respectively.

The pUC19-ELP1[K₁V₇F₁-72] and pUC19-ELP1 [K₁V₇F₁-144] plasmids produced in XL1-Blue were digested with PflM1 and Bgl1, and the ELP-containing fragments were ligated into the Sfi1 site of the pET24d-SD21 expression vector as described hereinabove to create pET24d-SD21-ELP1 [K₁V₇F₁-72] pET24d-SD21-ELP1 [K₁V₇F₁-144], respectively.

The pUC19-ELP2[60] and pUC19-ELP2[120] plasmids produced in XL1-Blue were digested with NcoI and HindIII, and the ELP-containing fragments were ligated into the NcoI and HindIII sites of the pET24d-SD21 expression vector as described hereinabove to create pET24d-SD21-ELP2[60], pET24d-SD21-ELP2[120], respectively.

The pUC19-ELP4[V-60] and pUC19-ELP4[V-120] plasmids produced in XL1-Blue were digested with NcoI and HindIII, and the ELP-containing fragments were ligated into the NcoI and HindIII sites of the pET24d-SD21 expression vector as described hereinabove to create pET24d-SD21-ELP4[V-60], pET24d-SD21-ELP4[V-120], respectively.

### Example 4

### Construction, Isolation and Purification of Various Fusion Proteins

Experiments have been conducted to show the use of various target proteins in forming ELP-containing fusion proteins and the inverse phase transition behavior exhibited by such fusion proteins. Specifically, the following thirty-six (36) ELP-containing fusion proteins were formed in E. coli by using known recombinant expression techniques consistent with the teachings and disclosures hereinabove:
Insulin A peptide and ELP1 [V-60] polypeptide with an enterokinase protease cleavage site therebetween (SEQ ID NO: 23);
Insulin A peptide and ELP1 [V₅A₂G₃-90] polypeptide with an enterokinase protease cleavage site therebetween (SEQ ID NO: 24);
Insulin A peptide and ELP1 [V-120] polypeptide with an enterokinase protease cleavage site therebetween (SEQ ID NO: 25);
Insulin A peptide and ELP1 [V₅A₂G₃-180] polypeptide with an enterokinase protease cleavage site therebetween (SEQ ID NO: 26);
T20 peptide and ELP1 [V-60] polypeptide with an enterokinase protease cleavage site therebetween (SEQ ID NO: 27);
T20 peptide and ELP1 [V₅A₂G₃-90] polypeptide with an enterokinase protease cleavage site therebetween (SEQ ID NO: 28);
T20 peptide and ELP1 [V-120] polypeptide with an enterokinase protease cleavage site therebetween (SEQ ID NO: 29);
T20 peptide and ELP1 [V-60] polypeptide with a thrombin protease cleavage site therebetween (SEQ ID NO: 30);
T20 peptide and ELP1 [V₅A₂G₃-90] polypeptide with a thrombin protease cleavage site therebetween (SEQ ID NO: 31);
T20 peptide and ELP1 [V-120] polypeptide with a thrombin protease cleavage site therebetween (SEQ ID NO: 32);
T20 peptide and ELP1 [V-60] polypeptide with a tobacco etch virus (TEV) protease cleavage site (cleavage between QS residues) therebetween (SEQ ID NO: 33);
T20 peptide and ELP1 [V₅A₂G₃-90] polypeptide with a TEV protease cleavage site (cleavage between QS residues) therebetween (SEQ ID NO: 34);
T20 peptide and ELP1 [V-120] polypeptide with a TEV protease cleavage site (cleavage between QS residues) therebetween (SEQ ID NO: 35);
T20 peptide and ELP1 [V-60] polypeptide with a TEV protease cleavage site (cleavage between QY residues) therebetween (SEQ ID NO: 36);
T20 peptide and ELP1 [V₅A₂G₃-90] polypeptide with a TEV protease cleavage site (cleavage between QY residues) therebetween (SEQ ID NO: 37);
T20 peptide and ELP1 [V-120] polypeptide with a TEV protease cleavage site (cleavage between QY residues) therebetween (SEQ ID NO: 38);
Interferon alpha 2B protein and ELP1 [V₅A₂G₃-90] polypeptide with a thrombin protease cleavage site therebetween (SEQ ID NO: 39);
Tobacco etch virus protease and ELP1 [V-60] polypeptide with a thrombin protease cleavage site therebetween (SEQ ID NO: 40);
Tobacco etch virus protease and ELP1 [V₅A₂G₃-90] polypeptide with a thrombin protease cleavage site therebetween (SEQ ID NO: 41);
Tobacco etch virus protease and ELP1 [V-120] polypeptide with a thrombin protease cleavage site therebetween (SEQ ID NO: 42);
Tobacco etch virus protease and ELP1 [V₅A₂G₃-180] polypeptide with a thrombin protease cleavage site therebetween (SEQ ID NO: 43);
Small heterodimer partner orphan receptor and ELP1 [V₅A₂G₃-90] polypeptide with a thrombin protease cleavage site therebetween (SEQ ID NO: 44);
Androgen receptor ligand binding domain and ELP1 [V₅A₂G₃-90] polypeptide with a thrombin protease cleavage site therebetween (SEQ ID NO: 45);
Androgen receptor ligand binding domain and ELP1 [V₅A₂G₃-180] polypeptide with a thrombin protease cleavage site therebetween (SEQ ID NO: 46);
Glucocorticoid receptor ligand binding domain and ELP1 [V₅A₂G₃-90] polypeptide with a thrombin protease cleavage site therebetween (SEQ ID NO: 47);
Estrogen receptor ligand binding domain and ELP1 [V₅A₂G₃-60] polypeptide with a thrombin protease cleavage site therebetween (SEQ ID NO: 48);
Estrogen receptor ligand binding domain and ELP1 [V₅A₂G₃-90] polypeptide with a thrombin protease cleavage site therebetween (SEQ ID NO: 49);
Estrogen receptor ligand binding domain and ELP1 [V₅A₂G₃-180] polypeptide with a thrombin protease cleavage site therebetween (SEQ ID NO: 50);
Estrogen receptor ligand binding domain and ELP1 [V₅A₂G₃-90] polypeptide with a TEV protease cleavage site (cleavage between QG residues) therebetween (SEQ ID NO: 51);
G protein alpha Q and ELP1 [V₅A₂G₃-90] polypeptide with a thrombin protease cleavage site therebetween (SEQ ID NO: 52);
G protein alpha Q and ELP1 [V₅A₂G₃-180] polypeptide with a thrombin protease cleavage site therebetween (SEQ ID NO: 53);
1-Deoxy-D-Xylulose 5-Phosphate reductoisomerase peptide and ELP1 [V₅A₂G₃-60] polypeptide with a thrombin protease cleavage site therebetween (SEQ ID NO: 54);
1-Deoxy-D-Xylulose 5-Phosphate reductoisomerase peptide and ELP1 [V₅A₂G₃-90] polypeptide with a thrombin protease cleavage site therebetween (SEQ ID NO: 55);
1-Deoxy-D-Xylulose 5-Phosphate reductoisomerase peptide and ELP1 [V₅A₂G₃-180] polypeptide with a thrombin protease cleavage site therebetween (SEQ ID NO: 56);
1-Deoxy-D-Xylulose 5-Phosphate reductoisomerase peptide and ELP1 [V₅A₂G₃-90] polypeptide with a TEV protease cleavage site (cleavage between QG residues) therebetween (SEQ ID NO: 57); and
G protein alpha S and ELP1 [V₅A₂G₃-90] polypeptide with a thrombin protease cleavage site therebetween (SEQ ID NO: 58).

All of the above-listed thirty-six ELP-containing fusion proteins were found to retain the inverse phase transition behavior of the corresponding ELP tags, and were successfully isolated and purified by using inverse transition cycling (ITC) techniques, according to the following experimental procedure:

Isolation and Purification of Fusion Proteins Containing Insulin A Peptide (InsA)

A single colony of E. coli strain BLR (DE3) (Novagen) containing the respective ELP-InsA fusion protein was inoculated into 5 ml CircleGrow (Q-BIOgene, San Diego, Calif.) supplemented with 100 μg/ml ampicillin (Sigma) and grown at 37° C. with shaking at 250 rpm for 5 hours. The 5 ml culture was then inoculated into a 500 ml culture and allowed to grow at 25° C. for 16 hours before inducing with 1 mM IPTG for 4 hours at 25° C. The culture was harvested and suspended in 40 ml 20 mM Tris-HCL pH 7.4, 50 mM NaCl, 1 mM DTT and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. Cell debris was removed by centrifugation at 20,000 g, 4° C. for 30 minutes.

Inverse phase transition was induced by adding NaCl to the cell lysate at room temperature to achieve a final concentration of 1.0 M therein, followed by centrifugation at 20,000 g for 15 minutes at room temperature. The resulting pellet contained the respective ELP-InsA fusion protein and non-specifically NaCl precipitated proteins.

The pellet was re-suspended in 40 ml ice-cold ml 20 mM Tris-HCL pH 7.4, 50 mM NaCl, 1 mM DTT and re-centrifuged at 20,000 g, 4° C. for 15 minutes to remove the non-specifically NaCl precipitated proteins. The inverse transition cycle was repeated two additional times to increase the purity of the respective ELP-InsA fusion protein and reduce the final volume to 0.5 ml.

Isolation and Purification of Fusion Proteins Containing T20 Peptide (T20)

A single colony of E. coli strain BLR (DE3) (Novagen) containing the respective ELP-T20 fusion protein was inoculated into 500 ml CircleGrow (Q-BIOgene, San Diego, Calif.) supplemented with 100 μg/ml ampicillin (Sigma) and grown at 37° C. with shaking at 250 rpm for 24 hours. The culture was harvested and suspended in 40 ml 50 mM Tris pH 8.0, 0.5 mM EDTA and 1 Complete Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. Cell debris was removed by centrifugation at 20,000 g, 4° C. for 30 minutes.

Inverse phase transition was induced by adding NaCl to the cell lysate at room temperature to achieve a final concentration of 1.0 M therein, followed by centrifugation at 20,000 g for 15 minutes at room temperature. The resulting pellet contained the respective ELP-T20 fusion protein and non-specifically NaCl precipitated proteins.

The pellet was re-suspended in 40 ml ice-cold ml 50 mM Tris pH 8.0, 0.5 mM EDTA and re-centrifuged at 20,000 g, 4° C. for 15 minutes to remove the non-specifically NaCl precipitated proteins. The inverse transition cycle was repeated two additional times to increase the purity of the respective ELP-T20 fusion protein and reduce the final volume to 5 ml.

Isolation and Purification of Fusion Protein Containing Interferon Alpha 2B Peptide (IFNA2)

A single colony of E coli strain BL21(DE3) TrxB⁻ (Novagen) containing the ELP-IFNα2 fusion protein and Codon Plus-RIL plasmid (Stratagene) was inoculated into 500 ml CircleGrow (Q-BIOgene, San Diego, Calif.) supplemented with 100 μg/ml ampicillin (Sigma), 25 ug/ml Chloramphenicol (Sigma) and incubated at 27° C. with shaking at 250 rpm for 48 hours. The culture was harvested and suspended in 50 mM Tris-HCL pH 7.4, 50 mM NaCl and 1 Complete EDTA free Protease inhibitor pellet (Roche, Indianapolis, Ind.). Cells were lysed by ultrasonic disruption on ice for 3 minutes, which consists of 10 seconds bursts at 35% power separated by 30 second cooling down intervals. Cell debris was removed by centrifugation at 20,000 g, 4° C. for 30 minutes.

Inverse phase transition was induced by adding NaCl to the cell lysate at room temperature to achieve a final concentration of 1.5 M, followed by centrifugation at 20,000 g for 15 minutes at room temperature. The resulting pellet contained the ELP-IFNα2 fusion protein and non-specifically NaCl precipitated proteins.

The pellet was re-suspended in 40 ml ice-cold 50 mM Tris-HCL pH 7.4 and 50 mM NaCl and re-centrifuged at 20,000 g, 4° C. for 15 minutes to remove the non-specifically NaCl precipitated proteins. The inverse transition cycle was repeated two additional times to increase the purity of the ELP-IFNA2 fusion protein and reduce the final volume to 5 ml.

Isolation and Purification of Fusion Proteins Containing Tobacco Etch Virus Protease (TEV)

A single colony of E. coli strain BL21 star or BRL(DE3) containing pET1Sb-SD5-ELP-TEV constructs and Codon Plus-RIL plasmid (Stratagene) was inoculated into 500 ml CircleGrow (Q-BIOgene, San Diego, Calif.) supplemented with 100 μg/ml ampicillin (Sigma), 25 ug/ml Chloramphenicol (Sigma) and incubated at 27° C. with shaking at 250 rpm for 48 hours. The culture was harvested and suspended in 50 mM Tris-HCL pH 8.0, 1 mM EDTA, 5 mM DTT, 10% glycerol and 1 mM PMSF. Cells were lysed by ultrasonic disruption on ice for 3 minutes, consisting of 10 seconds bursts at 35% power separated by 30 second cooling down intervals. Cell debris was removed by centrifugation at 20,000 g, 4° C. for 30 minutes.

Inverse phase transition was induced by adding NaCl to the cell lysate at room temperature to achieve a final concentration of 1.5 M, followed by centrifugation at 20,000 g for 15 minutes at room temperature. The resulting pellet contained the respective ELP-TEV fusion protein and non-specifically NaCl precipitated proteins.

The pellet was re-suspended in 40 ml ice-cold 50 mM Tris-HCL pH 8.0, 1 mM EDTA, 5 mM DTT, 10% glycerol and re-centrifuged at 20,000 g, 4° C. for 15 minutes to remove the non-specifically NaCl precipitated proteins. The inverse transition cycle was repeated two additional times to increase the purity of the respective ELP-TEV fusion protein and reduce the final volume to 1 ml.

Isolation and Purification of Fusion Protein Containing Small Heterodimer Partner Orphan Receptor (SHP)

A single colony of E. coli strain BL21 Star (DE3) containing the ELP-SHP fusion protein was inoculated into 500 ml CircleGrow (Q-BIOgene, San Diego, Calif.) supplemented with 100 μg/ml ampicillin (Sigma) and 10% sucrose and grown at 27° C. with shaking at 250 rpm for 48 hours. The culture was harvested and suspended in 50 mM Tris-HCL pH 8.0, 150 mM KCL, 1 mM DTT 1 mM EDTA and 1 Complete EDTA free Protease inhibitor pellet (Roche, Indianapolis, Ind.). Cells were lysed by ultrasonic disruption on ice for 3 minutes, which consistes of 10 seconds bursts at 35% power separated by 30 second cooling down intervals. DNA and RNA in the soluble lysate were further degraded by adding 2 μl Benzonase (Novagen) and incubating at 4° C. for 30 minutes. Cell debris was removed by centrifugation at 20,000 g, 4° C. for 30 minutes.

Inverse phase transition was induced by adding NaCl to the cell lysate at room temperature to achieve a final concentration of 1.5 M, followed by centrifugation at 20,000 g for 15 minutes at room temperature. The resulting pellet contained the ELP-SHP fusion protein and non-specifically NaCl precipitated proteins.

The pellet was re-suspended in 40 ml ice-cold 50 mM Tris-HCL pH 8.0, 150 mM KCL, 1 mM DTT1 mM EDTA, and 1% N-Octylglucoside and re-centrifuged at 20,000 g, 4° C. for 15 minutes to remove non-specific insoluble proteins. The temperature transition cycle was repeated two additional times to increase the purity of the ELP-SHP fusion protein and reduce the final volume to 2 ml.

Isolation and Purification of Fusion Proteins Containing Androgen Receptor Ligand Binding Domain (AR-LBD)

A single colony of E. coli strain BL21 Star (DE3) containing the respective ELP-AR-LBD fusion protein was inoculated into 500 ml CircleGrow (Q-BIOgene, San Diego, Calif.) supplemented with 100 μg/ml ampicillin (Sigma) and 10 μM DHT and grown at 27° C. with shaking at 250 rpm for 48 hours. The culture was harvested and suspended in 40 ml 50 mM Hepes pH 7.5, 150 mM NaCl, 0.1% N-Octylglycoside, 10% glycerol, 1 mM DTT, 1 μM DHT and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. DNA and RNA in the soluble sonicate were further degraded by adding 2 μl Benzonase (Novagen) and incubating at 4° C. for 30 minutes. Cell debris was removed by centrifugation at 20,000 g, 4° C. for 30 minutes.

Inverse phase transition was induced by adding NaCl to the cell lysate at room temperature to achieve a final concentration of 2.0 M, followed by centrifugation at 20,000 g for 15 minutes at room temperature. The resulting pellet contained the respective ELP-AR-LBD fusion protein and non-specifically NaCl precipitated proteins.

The pellet was re-suspended in 40 ml ice-cold 50 mM Hepes pH 7.5, 150 mM NaCl, 0.1% N-Octylglycoside, 10% glycerol, 1 mM DTT and 1 μM DHT and re-centrifuged at 20,000 g, 4° C. for 15 minutes to remove the non-specifically NaCl precipitated proteins. The inverse transition cycle was repeated two additional times to increase the purity of the respective ELP-AR-LBD fusion protein and reduce the final volume to 25 ml.

Isolation and Purification of Fusion Protein Containing Glucocorticoid Receptor Ligand Binding Domain (GR-LBD)

A single colony of E. coli strain BL21 Star (DE3) containing the ELP-GR-LBD fusion protein was inoculated into 500 ml CircleGrow (Q-BIOgene, San Diego, Calif.) supplemented with 100 μg/ml ampicillin (Sigma) and grown at 37° C. with shaking at 250 rpm for 24 hours. The culture was harvested and suspended in 50 mM Hepes pH 7.5, 150 mM NaCl, 1 mM DTT, 10% glycerol, 0.1% CHAPS and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. DNA and RNA in the soluble lysate were further degraded by adding 2 μl Benzonase (Novagen) and incubating at 4° C. for 30 minutes. Cell debris was removed by centrifugation at 20,000 g, 4° C. for 30 minutes.

Inverse phase transition was induced by adding NaCl to the cell lysate at room temperature to achieve a final concentration of 2.0 M, followed by centrifugation at 20,000 g for 15 minutes at room temperature. The resulting pellet contained the ELP-GR-LBD fusion protein and non-specifically NaCl precipitated proteins.

The pellet was re-suspended in 40 ml ice-cold in 50 mM Hepes pH 7.5,150 mM NaCl, 1 mM DTT, 10% glycerol, 0.1% CHAPS and re-centrifuged at 20,000 g, 4° C. for 15 minutes to remove the non-specifically NaCl precipitated proteins. The inverse transition cycle was repeated two additional times to increase the purity of the ELP-GR-LBD fusion protein and reduce the final volume to 10 ml.

Isolation and Purification of Fusion Proteins Containing Estrogen Receptor Ligand Binding Domain (ERα-LBD)

A single colony of E. coli strain BL21 Star (DE3) containing the respective ELP-ERa-LBD fusion protein was inoculated into 500 ml CircleGrow (Q-BIOgene, San Diego, Calif.) supplemented with 100 μg/ml ampicillin (Sigma), 10% sucrose (Sigma) and grown at 27° C. with shaking at 250 rpm for 48 hours. The culture was harvested and suspended in 40 ml 50 mM Tris-HCL pH 8.0,150 mM KCL, 1 mM EDTA, 1 mM DTT and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. DNA and RNA in the soluble lysate were further degraded by adding 2 μl Benzonase (Novagen) and incubating at 4° C. for 30 minutes. Cell debris was removed by centrifugation at 20,000 g, 4° C. for 30 minutes.

Inverse phase transition was induced by adding NaCl to the cell lysate at room temperature to achieve a final concentration of 1.5 M, followed by centrifugation at 20,000 g for 15 minutes at room temperature. The resulting pellet contained the respective ELP-ERα-LBD fusion protein and non-specifically NaCl precipitated proteins.

The pellet was re-suspended in 40 ml ice-cold 50 mM Tris-HCL pH 8.0, 150 mM KCL, 1 mM EDTA, 1 mM DTT and re-centrifuged at 20,000 g, 4° C. for 15 minutes to remove the non-specifically NaCl precipitated proteins. The inverse transition cycle was repeated two additional times to increase the purity of the respective ELP-ERα-LBD fusion protein and reduce the final volume to 10 ml.

Isolation and Purification of Fusion Proteins Containing G Protein Alpha 0 (Gαq)

A single colony of E. coli strain BL21 Star (DE3) containing the respective ELP-G_{αq} fusion protein was inoculated into 500 ml CircleGrow (Q-BIOgene, San Diego, Calif.) supplemented with 100 μg/ml ampicillin (Sigma) and 1 μM GDP and grown at 37° C. with shaking at 250 rpm for 24 hours. The culture was harvested and suspended in 40 ml 50 mM Hepes pH 7.5, 150 mM NaCl, 1.0% CHAPS, 10% glycerol, 1 mM DTT, 10 μM GDP and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. DNA and RNA in the soluble lysate were further degraded by adding 2 μl Benzonase (Novagen) and incubating at 4° C. for 30 minutes. Cell debris was removed by centrifugation at 20,000 g, 4° C. for 30 minutes.

Inverse phase transition was induced by adding NaCl to the cell lysate at room temperature to achieve a final concentration of 2.0 M, followed by centrifugation at 20,000 g for 15 minutes at room temperature. The resulting pellet contained the respective ELP-G_{αq} fusion protein and non-specifically NaCl precipitated proteins.

The pellet was re-suspended in 30 ml ice-cold 50 mM Hepes pH 7.5, 150 mM NaCl, 1.0% CHAPS, 10% glycerol, 1 mM DTT, 10 μM GDP and re-centrifuged at 20,000 g, 4° C. for 15 minutes to remove the non-specifically NaCl precipitated proteins. The inverse transition cycle was repeated two additional times to increase the purity of the respective ELP-G_{αq} fusion protein and reduce the final volume to 5 ml.

Isolation and Purification of Fusion Proteins Containing 1-Deoxy-D-Xylulose 5-Phosphate Reductoisomerase (DXR)

A single colony of E. coli strain BL21 Star (DE3) containing the respective ELP-DXR fusion protein was inoculated into 500 ml CircleGrow (Q-BIOgene, San Diego, Calif.) supplemented with 100 μg/ml ampicillin (Sigma), 1 mM MnCl₂ (VWR) and grown at 37° C. with shaking at 250 rpm for 24 hours. The culture was harvested and suspended in 40 ml 0.1M Tris pH 7.6, 1 mM DTT and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. DNA and RNA in the soluble lysate were further degraded by adding 2 μl Benzonase (Novagen) and incubating at 4° C. for 30 minutes. Cell debris was removed by centrifugation at 20,000 g at 4° C. for 30 minutes.

Inverse phase transition was induced by adding NaCl to the cell lysate at room temperature to achieve a final concentration of 2.0 M, followed by centrifugation at 20,000 g for 15 minutes at room temperature. The resulting pellet contained the respective ELP-DXR fusion protein and non-specifically NaCl precipitated proteins.

The pellet was re-suspended in 20 ml ice-cold 0.1 M Tris pH7.6, 1 mM DTT and centrifuged at 20,000 g, 4° C. for 15 minutes to remove the non-specifically NaCl precipitated proteins. The inverse transition cycle was repeated two additional times to increase the purity of the respective ELP-DXR fusion protein and reduce the final volume to 5 ml.

Isolation and Purification of Fusion Protein Containing G Protein Alpha S (Gas)

A single colony of E. coli strain BL21 Star (DE3) containing the ELP-G_{αs} fusion protein was inoculated into 500 ml CircleGrow (Q-BIOgene, San Diego, Calif.) supplemented with 100 μg/ml ampicillin (Sigma) and grown at 37° C. with shaking at 250 rpm for 24 hours. The culture was harvested and suspended in 40 ml PBS, 10% glycerol, 1 mM DTT and 1 Complete EDTA free Protease inhibitor pellet (Roche, Indianapolis, Ind.). Cells were lysed by ultrasonic disruption on ice for 3 minutes, which consisted of 10 seconds bursts at 35% power separated by 30 second cooling down intervals. DNA and RNA in the soluble lysate were further degraded by adding 2 μl Benzonase (Novagen) and incubating at 4° C. for 30 minutes. Cell debris was removed by centrifugation at 20,000 g, 4° C. for 30 minutes.

Inverse phase transition was induced by adding NaCl to the cell lysate at room temperature to a final concentration of 1.5 M, followed by centrifugation at 20,000 g for 15 minutes at room temperature. The resulting pellet contained the ELP-G_{αs} fusion protein and non-specifically NaCl precipitated proteins.

The pellet was re-suspended in 10 ml ice-cold PBS, 10% glycerol, 1 mM DTT and centrifuged at 20,000 g, 4° C. for 15 minutes to remove the non-specifically NaCl precipitated proteins. The inverse transition cycle was repeated two additional times to increase the purity of the ELP-G_{αs} fusion protein and reduce the final volume to 1 ml.

### REFERENCES

Throughout this specification various patent and non-patent references have been cited. The entire disclosure of each of these references is incorporated herein by reference, specifically including without limitation the following references:
1. Urry, D. W. Entropic elastic processes in protein mechanisms. 1. Elastic structure due to an inverse temperature transition due to internal chain dynamics. J. Prot. Chem. 7:1-34 (1988).
2. Urry, D. W. Free energy transduction in polypeptides and proteins based on inverse temperature transitions. Prog. Biophys. Molec. Biol. 57:23-57 (1992).
3. Urry, D. W. Physical chemistry of biological free energy transduction as demonstrated by elastic protein-based polymers. J. Phys. Chem. B 101: 11007-11028 (1997).
4. McPherson, D. T., Xu, J., and Urry, D. W. Product purification by reversible phase transition following Escherichia coli expression of genes encoding up to 251 repeats of the elastomeric pentapeptide GVGVP. Protein Expr Purif 7: 51-57 (1996).
5. Hoffman. A. S. Applications of thermally-reversible polymers and hydrogels in therapeutics and diagnostics. J. Controlled Release 6:297-305 (1987).
6. Chen, J. P. and Hoffman A. S. Protein-polymer conjugates II. Affinity precipitation separation of immunogammaglobulin by a poly(N-isopropylacrylamide)-protein A conjugate. Biomaterials 11:631-634 (1990).
7. Chilkoti, A., Chen, G-H., Stayton, P. S. and Hoffman, A. S. Site-specific conjugation of a temperature-sensitive polymer to a genetically-engineered protein. Bioconj. Chem. 5:504-507 (1994).
8. Nilsson, B., Forsberg, G., Moks, T., Hartmanis, M. and Uhlen, M. Fusion proteins in biotechnology and structural biology. Curr. Opin. Struct. Biol. 2:569:575 (1992).
9. Uhlén, M. and Moks, T. Gene fusions for purpose of expression: An introduction. Meth. Enzymol. 195:129-143 (1990).
10. Maina, C. V., Riggs, P. D., Grandea, A. G., III, Slatko, B. E., Moran, L. S., Tagliamonte, J. A., McReynolds, L. A. and diGuan, C. An Escherichia coli vector to express and purify foreign proteins by fusion to and separation from maltose binding protein. Gene 74:365-373 (1988).
11. Smith, D. B. and Johnson, K. S. Single-step purification of polypeptides expressed in Escherichia coli as fusions with glutathione S-transferase. Gene 67:31-40 (1988).
12. Tsao, K. W., deBarbieri, B., Hanspeter, M. and Waugh, D. W. A versatile plasmid expression vector for the production of biotinylated proteins by site-specific, enzymatic modification in Escherichiacoli. Gene 169: 59-64 (1996).
13. Smith, P. A., Tripp, B. C., DiBlasio-Smith, E. A., Lu, Z., LaValle, E. R. and McCoy, J. A. A plasmid expression system for quantitative in vivo biotinylation of thioredoxin fusion proteins in Escherichia coli. Nucl. Acid. Res. 26:1414-1420 (1998).
14. LaVallie, E. R., DiBlasio, E. A., Kovacic, S., Grant, K. L., Schendel, P. F. and McCoy, J. M. A thioredoxin gene fusion expression system that circumvents inclusion body formation in the E. coli cytoplasm. Bio/Technology 11:187-193 (1993).
15. Ong, E., Greenwood, J. M., Gilkes, N. R., Kilbum, D. G., Miller, R. C. Jr. and Warren, R. A. The cellulose-binding domains of cellulases: tools for biotechnology. Trends. Biotechnol. 7:239-243 (1989).

16. Smith, M. C., Furman, T. C., Ingolia, T. D. and Pidgeon, C. Chelating peptide-immobilized metal ion affinity chromatography. J. Biol. Chem. 263:7211-7215 (1988).
17. Kim, J-S. and Raines, R. T. Ribonuclease S-peptide as a carrier in fusion proteins. Prot. Sci. 2:348-356 (1993).
18. Su, X., Prestwood, A. K. and McGraw, R. A. Production of recombinant porcine tumor necrosis factor alpha in a novel E. coli expression system. Biotechniques 13:756-762 (1992).
19. Nilsson, J., StAhl, S., Lundeberg, J., Uhlen, M., and Nygren, P. A. Affinity Fusion Strategies for Detection, Purification, and Immobilization of Recombinant Proteins. Protein Expr Purif 11: 1-16 (1997).
20. Coligan, J. E., Dunn, B. M., Ploegh, H. L., Speicher, D. W. and Wingfield, P. T. Current protocols in protein science, John Wiley, (1995).
21. See Doi, Regulation of Gene Expression, Modern Microbial Genetics pages 15-39 (1991).
22. Hartmeier, W. Immobilized biocatalysts, Springer-Verlag, Berlin (1988).
23. Diamandis, E. P. and Christopoulos, T. K. Immunoassay, Academic Press, San Diego (1996).
24. Dehwirst, M. W. and Samulski, T. V. Hyperthermia in the treatment of cancer. Current Concepts 1-48 (1988).
25. Hauck, M. L., Dewhirst, M. W., Bigner, D. D. and Zalutsky, M. R. Local hyperthermia improves uptake of a chimeric monoclonal antibody in a subcutaneous xenograft model. Clin. Cancer Res. 3:63-70 (1997).
26. Cope, D. A., Dewhirst, M. W., Friedman, H. S., Bigner, D. D. and Zalutsky, M. R. Enhanced delivery of a monoclonal antibody F(ab′)2 fragment to subcutaneous human glioma xenografts using local hyperthermia. Cancer Res. 50:1803-1809 (1990).
27. Vertesy, L., Oeding, V., Bender, R., Zepf, K., and Nesemann, G. Tendamistat (HOE 467), a tight-binding alpha-amylase inhibitor from Streptomyces tendae 4158. Eur. J. Biochem 141:505-512 (1984).

28. Urry, D. W., Luan, C-H., Parker, T. M., Gowda, D. C., Prasad, K. U., Reid, M. C. and Safavy A. Temperature of polypeptide inverse temperature transition depends on mean residue hydrophobicity. J. Am. Chem. Soc. 113:4346-4348 (1991).
29. Urry, D. W., Trapane, T. L., and Prasad, K. U. Phase-structure transitions of the elastin polypentapeptide-water system within the framework of composition-temperature studies. Biopolymers 24:2345-2356 (1985).
30. Porath, J. Immobilized metal ion affinity chromatography. Prot. Expr. Purif. 3:262-282 (1992).
31. Holmgren, A. Thioredoxin. Annu. Rev. Biochem. 54:237-271 (1985).
32. Urry, D. W., Luan, C-H., Parker, T. M., Gowda, D. C., Prasad, K. U., Reid, M. C. and Safavy A. Temperature of polypeptide inverse temperature transition depends on mean residue hydrophobicity. J. Am. Chem. Soc. 113:4346-4348 (1991).
33. Urry, D. W., Trapane, T. L., and Prasad, K. U. Phase-structure transitions of the elastin polypentapeptide-water system within the framework of composition-temperature studies. Biopolymers 24:2345-2356 (1985).
34. Ausubel, F. M., Brent, R., Kingston, R. E., Moore, D. H., Seidman, J. G., Smith, J. A. and Struhl, K. Current protocols in molecular biology, John Wiley, New York (1995).
35. Smith, P. K., Krohn, R. I., Hermanson, G. T., Mallia, A. K., Gartner, F. H., Provenzano, M. D., Fujimoto, E. K., Goeke, N. M., Olson, B. J. and Kienk, D. C. Measurement of protein using bicinchonic acid. Anal. Biochem. 150:76-85 (1986).
36. Holmgren, A. and Bjornstedt, M. Enzymatic reduction-oxidation of protein disulfides by thioredoxin. Methods Enzymol. 107:295-300 (1984).
37. Meyer, D. and Chilkoti, A. Purification of Recombinant Proteins by Fusion with Thermally Responsive Polypeptides. Nat. Biotechnol. 17: 1112-1115 (1999).
38. Meyer, D. and Chilkoti, A. Genetically Encoded Synthesis of Protein-Based Polymers with Precisely Specified Molecular Weight and Sequence by Recursive Directional Ligation. Biomacromolecules (2001).
39. Coligan, J., Dunn, B., Ploegh, H., Speicher, D. and Wingfield, P. Current Protocols in Protein Science; John Wiley: New York, 1995.
40. Holmgren, A. Thioredoxin, Annu. Rev. Biochem. 54:237-271 (1985).
41. McPherson, D., Morrow, C., Minhan, D. Wu, J., Hunter, E., Urry, D. Production and purification of a recombinant elastomeric polypeptide, G-(VPGVG)₁₉-VPGV from Eschericia coli. Biotechnol. Prog. 8: 347-352 (1992).

1. A fusion protein exhibiting a phase transition, said fusion protein comprising:
(a) one or more biological molecules;
(b) one or more proteins exhibiting a phase transition joined to the biologically active molecule, wherein the one or more phase transition proteins are joined to the biological molecule(s) of (a); and
(c) optionally, a spacer sequence separating any of the phase transition protein(s) of (b) from any of the biological molecule(s) of (a),
wherein the one or more phase transition protein(s) of (b) comprise polymeric or oligomeric repeats of a polypeptide sequence selected from SEQ ID NO: 1-2 and 4-12.

2. The fusion protein of claim 1 wherein the biological molecule comprises a component selected from the group consisting of peptides, non-peptide proteins, lipids, oligonucleotides and carbohydrates.

3. The fusion protein of claim 1 wherein the biological molecule comprises a peptide.

4. The fusion protein of claim 1 wherein the biological molecule comprises a biologically active protein.

5. The fusion protein of claim 1 wherein the biological molecule comprises a therapeutic protein.

6. The fusion protein of claim 1 wherein the biological molecule comprises an enzyme useful in industrial biocatalysis.

7. The fusion protein of claim 1 wherein the biological molecule comprises a ligand-binding protein or an active fragment thereof having binding affinity to a biomolecule selected from the group consisting of small organic or inorganic molecules, proteins, peptides, single-stranded or double-stranded oligonucleotides, polynucleotides, lipids, and carbonhydrates.

8. The fusion protein of claim 7 wherein the ligand-binding protein or active fragment thereof has affinity for a protein of interest, and wherein upon binding to the protein of interest, the fusion protein retains some or all of its phase transition character.

9. The fusion protein of claim 1 wherein the phase transition is mediated by one or more means selected from the group comprising:
changing temperature;
changing pH;
addition of solutes and/or solvents,
side-chain ionization or chemical modification; and
changing pressure.

10. The fusion protein of claim 1 wherein the phase transition is mediated by means comprising raising temperature.

11. The fusion protein of claim 1 wherein the one or more protein(s) comprises protein exhibiting a β-turn.

12. The fusion protein of claim 1 wherein the one or more protein(s) comprises polymeric or oligomeric repeats of the pentapeptide Ile-Pro-Gly-X-Gly or Leu-Pro-Gly-X-Gly, wherein X is any natural or non-natural amino acid residue, and wherein X optionally varies among polymeric or oligomeric repeats.

13. The fusion protein of claim 12 wherein the X component(s) of the polymeric or oligomeric repeats comprise(s) a naturally-occurring amino acid residue.

14. The fusion protein of claim 12 wherein the X component(s) of the polymeric or oligomeric repeats comprise(s) a non-naturally-occurring amino acid residue.

15. The fusion protein of claim 12 wherein the X component(s) of the polymeric or oligomeric repeats comprise(s) one or more amino acid residues selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine residues.

16. The fusion protein of claim 12 wherein any two or more of the polymeric or oligomeric repeats are separated by one or more amino acid residues which do not eliminate the phase transition characteristic of the fusion protein.

17. The fusion protein of claim 1 comprising said spacer sequence.

18. The fusion protein of claim 17 wherein the spacer sequence comprises a proteolytic cleavage site.

19. The fusion protein of claim 1 wherein the fusion protein further comprises a signal peptide.

20. The fusion protein of claim 19 wherein the signal peptide is cleavable from the fusion protein by enzymatic cleavage.

21. The fusion protein of claim 19 wherein the signal peptide directs secretion of the fusion protein from the cell.

22. The fusion protein of claim 1 wherein the fusion protein or any of the biological molecule(s), protein(s), and spacer sequence when present, is recombinantly produced.

23. The fusion protein of claim 1 wherein the fusion protein or any of the biological molecule(s), protein(s), and spacer sequence when present, is synthetically produced.

24. A fusion protein exhibiting a phase transition, said fusion protein comprising:
(a) one or more proteins of interest;
(b) one or more β-turn protein(s) joined at a C- and/or N-terminus of any of the proteins of
(a); and
(c) optionally, a spacer sequence separating any of the protein(s) of (a) and/or (b).

25. The fusion protein of claim 24 wherein the phase transition is mediated by means comprising raising temperature.

26. A polynucleotide comprising a nucleotide sequence encoding the fusion protein of claim 24.

27. A polynucleotide comprising a nucleotide sequence encoding the fusion protein of claim 1.

28. An expression vector comprising the polynucleotide of claim 27.

29. A host cell transformed by the expression vector of claim 28, wherein said host cell expresses the fusion protein.

30. A method of producing one or more fusion proteins comprising:
(a) transforming a host cell with an expression vector comprising a polynucleotide comprising a nucleotide sequence encoding a fusion protein that exhibits a phase transition, wherein said fusion protein comprises: (i) one or more biological molecules; (ii) one or more proteins exhibiting a phase transition joined to the biologically active molecule, wherein the one or more phase transition proteins are joined to the biological molecule(s) of (i); and (iii) optionally, a spacer sequence separating any of the phase transition protein(s) of (ii) from any of the biological molecule(s) of (i), wherein the one or more phase transition protein(s) of (ii) comprise polymeric or oligomeric repeats of a polypeptide selected from SEQ ID NO: 1-2 and 4-12; and
(b) causing the host cell to express the fusion protein.

31. The method of claim 30 wherein the expressed fusion protein comprises a signal sequence directing secretion of the fusion protein from the cell.

32. The method of claim 30, further comprising the steps of:
(c) disrupting the cells to release the fusion protein; and
(d) isolating the protein by a method comprising raising temperature.

33. The method of claim 31, further comprising the step of isolating the secreted fustion protein by a method that comprises raising temperature.

34. A method of optimizing size of an ELP expression tag incorporated in a polynucleotide comprising a nucleotide sequence encoding a fusion protein exhibiting a phase transition, wherein the fusion protein comprises a protein of interest, said method comprising the steps of (i) forming a multiplicity of polynucleotides comprising a nucleotide sequence encoding a fusion protein exhibiting a phase transition, wherein each of said multiplicity of polynucleotides includes a different-sized ELP expression tag, (ii) expressing corresponding fusion proteins from said multiplicity of polynucleotides, (iii) determining a yield of the desired protein for each of said corresponding fusion proteins, (iv) determining size of particulates for each of said corresponding fusion proteins in solution as temperature is raised above Tₜ, and (v) selecting an optimized size ELP expression tag according to predetermined selection criteria for maximum recoverable protein of interest from among said multiplicity of polynucleotides.

35. A method of purification of fusion proteins to yield a protein of interest, comprising forming a polynucleotide comprising a nucleotide sequence encoding a fusion protein exhibiting a phase transition, expressing the fusion protein in culture, and subjecting a fusion protein-containing material from said culture to processing involving centrifugation and inverse transition cycling to recover said protein of interest.

36. The method of claim 35, comprising expressing the fusion protein in culture in a well of a microplate.

37. The method of claim 35, comprising processing the fusion protein-containing material from said culture in a well of a microplate.

38. A method of purifying a biomolecule of interest from a medium containing same, comprising adding to said medium an ELP-tagged purification agent that interacts with the biomolecule of interest to form a complex therewith, subjecting said medium containing said complex to ITC to insolubilize and aggregate the complex, and recovering aggregated complex comprising the biomolecule of interest from said medium.

39. The method of claim 38, wherein the biomolecule of interest is a therapeutic protein.

40. The method of claim 38, wherein the ELP-tagged purification agent comprises a ligand-binding protein having binding affinity to a biomolecule of interest selected from the group consisting of small organic or inorganic molecules, proteins, peptides, single-stranded or double-stranded oligonucleotides, polynucleotides, lipids, or carbonhydrates.

41. The method of claim 38, wherein the ELP-tagged purification agent comprises a binding moiety that binds to the biomolecule of interest in interaction therewith.

42. The method of claim 38, wherein said medium comprises a cell culture medium.

43. The method of claim 38, wherein said medium comprises an aqueous medium.

44. The method of claim 38, wherein said step of subjecting said medium containing said complex to ITC comprises varying a process condition of said medium selected from the group consisting of temperature, pH, and pressure.

45. The method of claim 38, wherein said step of subjecting said medium containing said complex to ITC comprises addition of a chemical reagent to said medium.

46. The method of claim 38, wherein said step of subjecting said medium containing said complex to ITC comprises addition of solute(s) and/or solvent(s) to said medium.

47. The method of claim 38, wherein said step of subjecting said medium containing said complex to ITC comprises addition of an ionic solute to said medium.

48. The method of claim 38, wherein said step of subjecting said medium containing said complex to ITC comprises addition of a salt to said medium.

49. The method of claim 38, wherein said step of subjecting said medium containing said complex to ITC comprises addition of NaCl to said medium.

50. The method of claim 38, further comprising recovering the biomolecule of interest from the aggregated complex comprising same.

51. The method of claim 50, wherein the recovery of the biomolecule of interest comprises decomplexing the biomolecule of interest from the ELP-tagged purification agent.

52. The method of claim 51, wherein said decomplexing comprises a decomplexing step selected from the group consisting of: heating the complex; solvating the complex in a solvent medium effecting disengagement of the biomolecule of interest from the ELP-tagged purification agent; and varying the pH environment of the complex.

53. A method of producing a purified protein of interest, comprising:
providing a fusion protein comprising the protein of interest and an ELP tag, wherein the fusion protein contains at least one cleavage site that is cleavable to yield the protein of interest as a cleavage product;
contacting the fusion protein with an ELP-tagged cleavage agent that is effective to cleave said cleavage site, thereby yielding said protein of interest as a cleavage product, in a cleavage product mixture comprising said ELP tag, any uncleaved fusion protein, and said ELP-tagged cleavage agent;
subjecting the cleavage product mixture to ITC to insolubilize and aggregate each of said ELP tag, any uncleaved fusion protein and ELP-tagged cleavage agent; and
recovering the protein of interest.

54. The method of claim 53, wherein said step of subjecting said cleavage product mixture to ITC comprises varying a process condition of said cleavage product mixture selected from the group consisting of temperature, pH, and pressure.

55. The method of claim 53, wherein said step of subjecting said cleavage product mixture to ITC comprises addition of a chemical reagent to said cleavage product mixture.

56. The method of claim 53, wherein said step of subjecting said cleavage product mixture to ITC comprises addition of solute(s) and/or solvent(s) to said cleavage product mixture.

57. The method of claim 38, wherein said step of subjecting said cleavage product mixture to ITC comprises addition of an ionic solute to said cleavage product mixture.

58. The method of claim 38, wherein said step of subjecting said cleavage product mixture to ITC comprises addition of a salt to said cleavage product mixture.

59. The method of claim 38, wherein said step of subjecting said cleavage product mixture to ITC comprises addition of NaCl to said cleavage product mixture.

60. A method of production of a protein of interest, comprising expressing the protein of interest in a culture medium, binding the expressed protein of interest to an ELP tag, and recovering the expressed protein of interest bound to the ELP tag by a recovery process comprising ITC.

61. The method of claim 60, wherein the protein of interest is a therapeutic protein.

62. The method of claim 60, wherein the ELP tag is bound to the protein of interest by a ligand-binding protein specific for the protein of interest.

63. A method of automated high-throughput protein purification, comprising
providing a multi-well filter block,
introducing to wells of the multi-well filter block transformed cells expressing fusion proteins including a protein of interest and an ELP tag,
incubating said cells to express said fusion proteins,
lysing said cells in said wells,
heating the multi-well filter block to precipitate said fusion proteins, and
removing cell debris from said fusion proteins.

64. A method of protein production in which a protein of interest is produced as a component of an ELP fusion protein and said ELP fusion protein is subjected to ITC for recovery thereof under ITC conditions effective therefor, comprising monitoring recovery of said ELP fusion protein, and responsively adjusting said ITC conditions to maintain a predetermined level of said recovery of said ELP fusion protein.

65. The process of claim 64, wherein said ITC conditions comprise turbidity of an aqueous medium containing said ELP fusion protein being subjected to ITC.

66. An ELP fusion protein containing a cleavage site that is selected from the group consisting of a photolabile cleavage site, a thermally labile cleavage site, and a cleavage site cleavable by exposure to light, electromagnetic radiation, change of pH, or change of temperature.

67. An ELP fusion protein comprising a signal peptide sequence and/or a heat shock protein sequence.

68. A method of protein production, comprising expressing in an expression medium an ELP fusion protein including a protein of interest, recovering the ELP fusion protein from the expression medium by a recovery process including thermally-mediated ITC, and subjecting the recovered ELP fusion protein to a non-enzymatic separation of the protein of interest from the ELP fusion protein.

69. The method of claim 68, wherein the non-enzymatic separation comprises thermoscission of the ELP fusion protein.

70. The method of claim 68, wherein the non-enzymatic separation comprises a radiation-mediated scission of the ELP fusion protein.

71. The method of claim 68, wherein the protein of interest comprises a therapeutic protein.

72. An ELP fusion protein including an ELP moiety and a protein of interest, wherein the ELP fusion protein comprises a cleavage moiety between the ELP moiety and the protein of interest, and the cleavage moiety includes a cleavage site that is cleavable by a modality selected from the group consisting of thermolysis, photolysis, shear-mediated lysis, pH change, and exposure to an ultrasonic or predetermined frequency field providing energy effective for cleavage.

73. A prokaryotic cell transformed to express an ELP fusion protein.

74. An eukaryotic cell transformed to express an ELP fusion protein.

75. A thermophilic prokaryotic cell transformed to express an ELP fusion protein.

76. A mesophilic prokaryotic cell transformed to express an ELP fusion protein.

77. A thermotolerant prokaryotic cell transformed to express an ELP fusion protein.

78. A thermotolerant prokaryotic cell transformed to express an ELP fusion protein, wherein the ELP fusion protein comprises an ELP moiety and a protein of interest, and a cleavage moiety including a thermally labile bond cleavable at a temperature above temperature of ITC phase transition of the ELP fusion protein.

79. The thermotolerant prokaryotic cell of claim 78, wherein said cell is a thermophilic prokaryotic cell.

80. The thermotolerant prokaryotic cell of claim 78, wherein said cell is a mesophilic prokaryotic cell.

81. The thermotolerant prokaryotic cell of claim 78, wherein said ELP fusion protein comprises a signal peptide sequence mediating secretion of the ELP fusion protein from the cell.

82. The thermotolerant prokaryotic cell of claim 78, wherein said cell further comprises heat shock proteins.

83. A protein production method, comprising:
providing cells in culture, wherein said cells have been transformed to express an ELP fusion protein including a thermally labile bond between an ELP moiety and a protein of interest in said ELP fusion protein;
incubating the cells to express said ELP fusion protein;
releasing said ELP fusion protein from said cells;
subjecting the ELP fusion protein to a purification process including ITC processing at a first elevated temperature;
heating the ELP fusion protein from the purification process to temperature above said first elevated temperature to thermally break the thermally labile bond, and yield said ELP moiety and said protein of interest as thermolysis products; and
subjecting said thermolysis products to ITC processing to recover said protein of interest.

84. The method of claim 83, wherein said cells comprise thermotolerant cells.

85. The method of claim 83, wherein said cells comprise thermophilic prokaryotic cells.

86. The method of claim 83, wherein said cells comprise mesophilic prokaryotic cells.

87. A method of protein production including culturing transformed cells for expression of secretory ELP fusion proteins and secretion of ELP fusion proteins from the cells, and subjecting the secreted ELP fusion proteins to ITC at elevated temperature for purification thereof, comprising inducing heat shock protein production in the cells.

88. A method of producing a protein of interest including subjecting an ELP fusion protein comprising the protein of interest, to ITC for recovery of the ELP fusion protein, wherein said ITC effects aggregation of desolubilized particles of the ELP fusion protein, comprising monitoring size of aggregates of the desolubilized particles of the ELP fusion protein, and responsively adjusting temperature so that said aggregates are maintained in an aggregate size regime to achieve a predetermined yield of the protein of interest.

89. The method of claim 88, wherein said monitoring of size of aggregates comprises monitoring turbidity, opacity, light scattering or light attenuation of a medium containing said ELP fusion protein.

90. A method of protein production including recovery of ELP fusion protein material from a medium containing same by a recovery process including ITC, wherein said ELP fusion protein material comprises a population of ELP fusion proteins having ELP tags of different lengths, in mixture with one another, thereby maintaining stable yields, separability and aggregate size of the ELP fusion protein material, whereby perturbations of temperature or other environmental conditions do not cause gross deviations in the level of recovery of the purified protein of interest.

91. The method of claim 90, wherein said population is adjusted by addition of one or more differently ELP-sized sub-populations of ELP fusion proteins so that the relative proportions of said differently ELP-sized sub-populations of fusion proteins relative to one another are maintained for achieving a predetermined level of recovery of the purified protein of interest.

92. A method of protein purification, comprising expressing a fusion protein including a protein of interest and an affinity tag, and contacting the fusion protein, in a medium containing same, with an ELP-protein whose protein moiety binds to said affinity tag, thereby forming a protein complex comprising said fusion protein and ELP-protein, and subjecting the protein complex to ITC to recover same from said medium.

93. The method of claim 92, wherein said medium comprises a culture medium.

94. The process of claim 92, wherein the affinity tag is selected from the group consisting of maltose binding protein (MBP), glutathione S-transferase (GST), biotin carboxyl carrier protein, thioredoxin, cellulose binding domain, oligohistidine, S-peptide, and FLAG peptide.

95. A method of protein production including expression of an ELP fusion protein including a protein of interest and a cleavage site that is enzymatically cleavable to release the protein of interest from the ELP fusion protein, said method comprising:
subjecting the ELP fusion protein to ITC for purification thereof,
contacting the purified ELP fusion protein with an ELP-tagged enzyme effective for enzymatically cleaving ELP fusion protein to release the protein of interest from the ELP fusion protein and produce a cleavage mixture including the protein of interest, ELP, uncleaved fusion protein, and the ELP-tagged enzyme,
subjecting the cleavage mixture to ITC to insolubilize ELP, uncleaved fusion protein, and the ELP-tagged enzyme, and
recovering the protein of interest from the cleavage mixture.

96. The method of claim 95, wherein the protein of interest is a therapeutic protein.

97. The method of claim 96, wherein said therapeutic protein comprises a protein selected from the group consisting of erythropoietins, inteferons, insulin, monoclonial antibodies, blood factors, colony stimulating factors, growth hormones, interleukins, growth factors, therapeutic vaccines, calcitonins, tumor necrosis factors (TNF), and enzymes.

98. The method of claim 95, wherein the cleavage site of the ELP fusion protein comprises a cleavage site selected from the group consisting of: -Pro-Val-▾-Gly-Pro-(Collagenase); -Asp-Asp-Asp-Lys-▾(Enterokinase); -Ile-Glu-Gly-Arg-▾(Factor Xa); -Gly-Pro-Arg-▾(Thrombin); -Glu-Asn-Leu-Tyr-Phe-Gln-▾(Tobacco etch virus protease); -Arg-▾(Trypsin); -Arg-▾(Clostripain); -Gly-Ala-His-Arg-▾(Ala⁶⁴-Subtilisin); Factor XIII cleavage sites and intein cleavage sites.

99. A method of protein production including expression of an ELP fusion protein including a protein of interest and a cleavage site that is photolytically cleavable to release the protein of interest from the ELP fusion protein, said method comprising
subjecting the ELP fusion protein to ITC for purification thereof,
contacting the purified ELP fusion protein with light that is effective for photolytically cleaving ELP fusion protein to release the protein of interest from the ELP fusion protein and produce a cleavage mixture including the protein of interest, ELP, and uncleaved fusion protein,
subjecting the cleavage mixture to ITC to insolubilize ELP and uncleaved fusion protein, and
recovering the protein of interest from the cleavage mixture.

100. A method of protein production including expression of an ELP fusion protein including a protein of interest and a chemical cleavage site that is chemically cleavable to release the protein of interest from the ELP fusion protein, said method comprising
subjecting the ELP fusion protein to ITC for purification thereof,
contacting the purified ELP fusion protein with a chemical cleavage reagent for chemically cleaving ELP fusion protein to release the protein of interest from the ELP fusion protein and produce a cleavage mixture including the protein of interest, ELP, and uncleaved fusion protein,
subjecting the cleavage mixture to ITC to insolubilize ELP and uncleaved fusion protein, and
recovering the protein of interest from the cleavage mixture.

101. The method of claim 100, wherein said chemical cleavage reagent is selected from the group consisting of cyanogen bromide, N-chlorosuccinimide, BNPS-skatole, acids, and hydroxylamine.

102. The method of claim 100, wherein said chemical cleavage site comprises an acid-cleavable -Asp-Pro-cleavage site, and wherein the purified ELP fusion protein is contacted with acid that is effective for cleaving the ELP fusion protein to release the protein of interest from the ELP fusion protein.

103. The method of claim 100, wherein said chemical cleavage site comprises methionine residue, and wherein the purified ELP fusion protein is contacted with cyanogens bromide for cleaving the ELP fusion protein to release the protein of interest from the ELP fusion protein.

104. The method of claim 100, wherein said chemical cleavage site comprises tryptophan residue, and wherein the purified ELP fusion protein is contacted with N-chlorosuccinimide for cleaving the ELP fusion protein to release the protein of interest from the ELP fusion protein.

105. The method of claim 100, wherein said chemical cleavage site comprises tryptophan residue, and wherein the purified ELP fusion protein is contacted with BNPS-skatole for cleaving the ELP fusion protein to release the protein of interest from the ELP fusion protein.

106. The method of claim 100, wherein said chemical cleavage site comprises an -Asn-Gly-cleavage site, and wherein the purified ELP fusion protein is contacted with hydroxylamine for cleaving the ELP fusion protein to release the protein of interest from the ELP fusion protein.

107. A method for producing a fusion protein including a therapeutic protein and an ELP tag, comprising:
(i) expressing the fusion protein in a transformed host cell;
(ii) secreting the fusion protein from the host cells, or alternatively disrupting the host cells to release the fusion protein;
(iii) aggregating the fusion protein by a method that comprises ITC;
(iv) concentrating the aggregated fusion protein by centrifugation;
(v) discarding the supernatant and resolubilizing the pelleted fusion protein;
(vi) adding an enzyme to cleave the therapeutic protein from its ELP-tag;
(vii) aggregating free ELP-tag by a method that comprises ITC;
(viii) concentrating the aggregated free ELP-tag by centrifugation; and
(ix) recovering supernatant containing the therapeutic protein.

108. The method of claim 107, wherein said therapeutic protein comprises a protein selected from the group consisting of erythropoietins, inteferons, insulin, monoclonial antibodies, blood factors, colony stimulating factors, growth hormones, interleukins, growth factors, therapeutic vaccines, calcitonins, tumor necrosis factors (TNF), and enzymes.

109. The method of claim 53, wherein the protein of interest comprises two or more cleavage sites.

110. The method of claim 109, wherein the protein of interest comprises multiple proteins of interest, wherein the protein of interest is sequentially fractionated by cleavage and ITC to sequentially yield said multiple proteins of interest.

111. A method of conducting a biocatalytic reaction in a reaction zone, comprising utilizing a biocatalyst to catalyze the reaction, wherein the biocatalyst comprises an ELP fusion protein, and removing the biocatalyst from the reaction zone by ITC.

112. The method of claim 11, wherein the reaction zone is within a bioreactor.

113. The method of claim 111, wherein the ELP fusion protein is solubilized in a reaction medium in the reaction zone during the biocatalytic reaction to effect catalysis of the reaction.

114. The method of claim 111, wherein the ELP fusion protein is added to the reaction zone at temperature above Tₜ of the ELP fusion protein, and temperature in the reaction zone is decreased to below said Tₜ to effect catalysis of the reaction.

115. The method of claim 111, wherein cells transformed to express the ELP fusion protein are disposed in the reaction zone, and the ELP fusion protein is expressed in situ in the reaction zone from said cells, and secreted therefrom into a reaction medium in the reaction zone.

116. The method of claim 115, wherein the reaction medium comprises an aqueous medium.

117. The method of claim 115, wherein the reaction medium comprises a culture medium containing said transformed cells.

118. The method of claim 111, wherein said biocatalytic reaction produces a therapeutic or diagnostic agent.

119. A method of producing one or more fusion proteins comprising:
(a) transforming a host cell with an expression vector comprising a polynucleotide comprising a nucleotide sequence encoding a fusion protein that exhibits a phase transition, wherein said fusion protein comprises: (i) one or more biological molecules; (ii) one or more proteins exhibiting a phase transition joined to the biologically active molecule, wherein the one or more phase transition proteins are joined to the biological molecule(s) of (i); and (iii) optionally, a spacer sequence separating any of the phase transition protein(s) of (ii) from any of the biological molecule(s) of (i); and
(b) causing the host cell to express the fusion protein.
